(19) United States
(12) Patent Application Publication
(10) Pub. No.: US 2012/0156676 A1
(43) Pub. Date: Jun. 21, 2012

(54) SINGLE NUCLEOTIDE POLYMORPHISMS IN BRCA1 AND CANCER RISK

(76) Inventors: Joanne B. Weidhaas, Westport, CT (US); Cory Pelletier, New Haven, CT (US)

(21) Appl. No.: 13/379,995

(22) PCT Filed: Jun. 25, 2010
(86) PCT No.: PCT/US2010/040105

§ 371 Date: Mar. 6, 2012

Related U.S. Application Data
(63) Continuation-in-part of application No. 61/220,342, filed on Jun. 25, 2009.
(60) Provisional application No. 61/220,342, filed on Jun. 25, 2009.

(51) Int. Cl. C12Q 1/68; C07H 21/04

(52) U.S. Cl. 435/6.11; 536/23.1

(57) ABSTRACT

The invention provides methods for identifying mutations, such as single nucleotide polymorphisms (SNPs), within breast and ovarian cancer associated genes that modify the binding efficacy of microRNAs (miRNAs). In a preferred embodiment, methods of the invention identify a SNP that decreases expression of the BRCA1 gene by increasing or decreasing the binding efficacy of at least one miRNA. Alteration of miRNA binding to BRCA1 by the introduction of SNPs within miRNA binding sites modulates or decreases BRCA1 expression, ultimately leading to the unregulated cell proliferation of a breast or ovarian cancer cells.

### RELATED APPLICATIONS

This application is related to provisional application U.S. Ser. No. 61/220,342, filed Jun. 25, 2009, the contents of which are herein incorporated by reference in their entirety.

### GOVERNMENT SUPPORT

This invention was made with Government support under Grant Nos. CA124484 and CA131301-01A1, both of which were awarded by the National Institutes of Health. The Government has certain rights in the invention.

### FIELD OF THE INVENTION

This invention relates generally to the fields of cancer and molecular biology. The invention provides compositions and methods for predicting the increased risk of developing cancer.

### BACKGROUND OF THE INVENTION

Even though there has been progress in the field of cancer detection, there still remains a need in the art for the identification of new genetic markers for a variety of cancers that can be easily used in clinical applications. To date, there are relatively few options available for predicting the risk of developing cancer.

### SUMMARY OF THE INVENTION

The methods of the invention provide means to not only identify polymorphisms in breast and ovarian cancer genes that could potentially modify the ability of miRNAs to bind targets, but also to assess the effect of these SNPs on target gene regulation and the risk of breast and ovarian cancer. These methods are used to identify patients with increased breast and ovarian cancer risk, who have previously been unrecognized. Of particular relevance are the identification and characterization of SNPs that occur within the region surrounding and including the BRCA1 gene or a messenger RNA (mRNA) transcript thereof using the methods of the invention.

The invention provides a method for identifying single nucleotide polymorphisms (SNPs) in the 3′ untranslated region (UTR) of breast and ovarian cancer associated genes that could potentially modify the ability of microRNAs (miRNAs) to bind. In a preferred embodiment, the breast and ovarian cancer associated gene is BRCA1, including the BRCA1 gene itself, the surrounding areas within the genome, BRCA1 regulatory elements and/or a messenger RNA (mRNA) transcript thereof. Several art-recognized databases are used to computationally identify SNPs of interest, including but not limited to, HapMap (The International HapMap Project. Nature, 2003. 426, 789-96), dbSNP (Sherry, S. T. et al. Genome Res 1999. 9, 677-9), and the Ensembl Project database (available at http://www.ensembl.org), as well as specialized algorithms, such as PicTar (Landi, D. et al. DNA Cell Biol (2007)), TargetScan (Lewis, B. P. et al. Cell 2005. 120, 15-20), miRanda (John, B. et al. PLoS Biol 2004. 2, e363), miRNA.org (Betel, D. et al. Nucleic Acids Res 2008. 36, D149-53), and MicroInspector (Rusinov, V. et al. Nucleic Acids Res 2005. 33, W696-700) to identify miRNA binding sites.

The invention also provides a method for identifying breast and ovarian tumors, adjacent normal tissue (when available) and normal tissue samples to evaluate sequence variations in miRNA complimentary sites. In a preferred embodiment of this method, the BRCA1 gene, or an mRNA transcript thereof, contains the miRNA complimentary site. In certain embodiments of the invention, the adjacent normal tissue is used to confirm if variations are germ line SNPs. Alternatively, or in addition, 3′ UTR mutations that are not germ line are also analyzed for clinical significance.

Moreover, the invention provides a method to assess the effect of identified SNPs on target gene regulation in vitro. In a preferred aspect of this method, the identified SNPs are contained within the BRCA1 gene or an mRNA transcript thereof. In another preferred aspect of this method, the identified SNPs are contained within the 3′UTR of the BRCA1 mRNA. In certain aspects of the invention SNPs are evaluated using a cell culture system and the luciferase assay to measure expression levels (Chin, L. J. et al. Cancer Res 2008. 68, 8535-40; Johnson, S. M. et al. Cell 2005. 120, 635-47). To generate a wild-type 3′UTR, polymerase chain reaction (PCR) is used to amplify human genomic DNA from a cell line. To construct the variant sequence, site-directed mutagenesis is used (Johnson, S. M. et al. Cell 2005. 120, 635-47). These constructs are then cloned into luciferase reporters. Finally, reporter expression is quantified by using GraphPad Prism (Chin, L. J. et al. Cancer Res 2008. 68, 8535-40).

The invention further provides methods to assess the risk of developing breast and ovarian cancer. In one aspect of this method, the prevalence of a SNP of interest is compared in a sample cancer population with respect to the expected prevalence in World populations. In a preferred embodiment of this method, the SNP of interest is contained within the BRCA1 gene or an mRNA transcript thereof. For novel SNPs, a TaqMan PCR assay (Applied Biosystems) can be created for allelic discrimination prior to comparison to world populations. In other embodiments of the methods provided herein, SNPs of interest are compared to breast and ovarian cancer case controls to determine the increased risk associated with the SNP of developing breast and/or ovarian cancer with respect to the general population and those individuals who do not carry the SNP.

Specifically, the invention provides an isolated and purified BRCA1 haplotype including at least one single nucleotide polymorphism (SNP), wherein the presence of the SNPs increases a subject's risk of developing breast or ovarian cancer. Haplotypes of the invention are isolated and purified genomic or cDNA sequences. Moreover, haplotypes are isolated, purified, and, optionally, amplified sequences. Genomic DNA and cDNA sequences from which haplotype sequences are isolated are obtained from biological samples including, bodily fluids and tissue. Most commonly the DNA sequences from which the haplotypes are derived are isolated from, for example, blood or tumor samples collected from normal or test subjects. In one aspect of this haplotype, each of the SNPs alters the activity of one or more miRNA(s). In another aspect of this haplotype, each of the SNPs increases or decreases the activity of one or more miRNA(s). In certain aspects, the SNP increases or decreases the binding efficacy of one or more miRNAs to a miRNA binding site. Alternations of miRNA binding efficacy increase or decrease the expression of BRCA1, and in preferred embodiments, the alterations of miRNA binding efficacy decrease BRCA1 expression. A SNP may be located in a noncoding or a coding region of the BRCA1 gene, surrounding genes, and inter- or intra-genic sequences of the genome that regulate, alter, increase, or decrease BRCA1 expression. SNPs located in noncoding as well as coding regions of the BRCA1 gene are located in miRNA binding sites, and consequently, inhibit the activity of one or more miRNA(s). In certain embodiments of this haplotype, the SNP is selected from the group consisting of rs9911630, rs12516, rs8176318, rs3092995, rs1060915, rs799912, rs9908805, and rs17599948. In a preferred embodiment, the SNP is selected from the group consisting of rs12516, rs8176318, rs3092995, rs1060915, and rs799912. In the most selective embodiment, the haplotype comprises rs8176318 and rs1060915. Alternatively, the SNP is either rs8176318 or rs1060915.

The haplotypes described herein increase a subject's risk of developing breast or ovarian cancer. Although all subtypes of breast and ovarian cancer are encompassed by the invention, specific subtypes of breast cancer that are commonly contemplated are triple negative (TN) (ER/PR/HER2 negative), estrogen receptor positive (ER+), estrogen and progesterone receptor positive (ER+/PR+), and human epidermal growth factor receptor 2 positive (HER2+). In a preferred embodiment, the rare haplotypes described herein are most frequently associated with TN breast cancer. Without wishing to be bound by theory, among the hormone-receptor specific breast cancer subtypes listed herein, TN breast cancer is least often associated with sporadic causes, and, therefore, the most likely to be inherited. TN breast cancer is also positively associated with haplotypes that contain the rs8176318 SNP and/or rs1060915, particularly in African American subjects.

The invention encompasses all disclosed haplotypes. Preferred haplotypes include the “rare” haplotypes described herein: GGACGCTA (SEQ ID NO: 6), GGCCGCTA (SEQ ID NO: 9), GGCCGCTG (SEQ ID NO: 10), GGACGCTG (SEQ ID NO: 21), or GAACGTTG (SEQ ID NO: 26).

The invention further provides a BRCA1 polymorphic signature that indicates an increased risk for developing breast or ovarian cancer, the signature including the determination of the presence or absence of the following single nucleotide polymorphisms (SNPs) rs8176318 and rs1060915, wherein the presence of these SNPs indicates an increased risk for developing breast or ovarian cancer. In certain embodiments, the signature further includes the determination of the presence or absence of at least one SNP selected from the group consisting of rs12516, rs3092995, and rs799912. Alternatively, or in addition, the signature includes the determination of the presence or absence of at least one SNP selected from the group consisting of rs9911630, rs9908805, and rs17599948. In one aspect of this signature, rs8176318, rs1060915, rs12516, rs3092995, rs799912, rs9911630, rs9908805, or rs17599948 alter the binding efficacy of at least one microRNA (miRNA). Alternatively, rs8176318, rs1060915, rs12516, rs3092995, rs799912, rs9911630, rs9908805, and rs17599948 increase or decrease the binding efficacy of at least one microRNA (miRNA). The at least one miRNA is any human miRNA provided by, for instance, miRBase (publicly available at http://www.mirbase.org/). In certain embodiments, the miRNA is miR-19a, miR-18b, miR-19b, miR-146-5p, miR-18a, miR-365, miR-210, miR-7, miR-151-3p, miR-1180. Preferably, the miRNA is miR-7.

In other embodiments, this signature further includes the identification of the presence or absence of at least one SNP in the BRCA1 gene that decreases the binding efficacy of one or more microRNAs. The at least one SNP may occur within a coding or a non-coding region. Exemplary non-coding regions include, but are not limited to, the 3′ untranslated region (UTR), an intron, an intergenic region, a cis-regulatory element, promoter element, enhancer element, or the 5′ untranslated region (UTR). A non-limiting example of a coding region is an exon.

The signatures described herein determine a subject's risk of developing breast or ovarian cancer. Although all subtypes of breast and ovarian cancer are encompassed by the invention, specific subtypes of breast cancer that are commonly contemplated are triple negative (TN) (ER/PR/HER2 negative), estrogen receptor positive (ER+), estrogen and progesterone receptor positive (ER+/PR+), and human epidermal growth factor receptor 2 positive (HER2+). In a preferred embodiment, the signatures described herein are used to determine the risk of developing TN breast cancer, particularly in African American subjects.

The invention also provides a method of identifying a SNP that decreases expression of the BRCA1 gene and increases a subject's risk of developing breast or ovarian cancer, including: (a) obtaining a sample from a test subject; (b) obtaining a control sample; (c) determining the presence or absence of a SNP in at least one miRNA binding site within a DNA sequence from the test sample; and (d) evaluating the binding efficacy of at least one miRNA to the at least one miRNA binding site containing the SNP compared to the binding efficacy of the miRNA to the same miRNA binding site in corresponding DNA sequence from the control sample, wherein the presence of a statistically-significant alteration in the binding efficacy of the at least one miRNA to the corresponding binding site(s) between the control and test samples indicates that the presence or absence of the SNP inhibits miRNA-mediated protection or increases miRNA-mediated repression of BRCA1 gene expression, thereby identifying a SNP that also increases a subject's risk of developing breast or ovarian cancer. The presence of a statistically-significant increase or decrease in the binding efficacy of the at least one miRNA to the corresponding binding site(s) between the control and test samples indicates that the presence or absence of the SNP inhibits miRNA-mediated protection or increases repression of BRCA1 gene expression. In certain embodiments of this method, the test subject has been diagnosed with breast or ovarian cancer. In contrast, the control sample is obtained from a subject who has not been diagnosed with any cancer. Moreover the control sample can also be a control value retrieved from a database or clinical study. Binding efficacy of the miRNA to the binding site in the DNA sequence from the test or control sample is evaluated in vivo, in vitro or ex vivo.

The invention provides a method of identifying a SNP that decreases expression of the BRCA1 gene and increases a subject's risk of developing breast or ovarian cancer, including: (a) obtaining a sample from a test subject; (b) determining the presence or absence of a SNP in at least one miRNA binding site in a DNA sequence from the test sample; and (c) evaluating the prevalence of the SNP within a breast or ovarian cancer population with respect to the expected prevalence of the SNP in one or more world population(s), wherein a statistically-significant increase in the presence or absence of the SNP in the tumor sample compared to the one or more world populations indicates that the SNP is positively associated with an increased risk of developing breast or ovarian cancer and wherein the presence or absence of the SNP within at least one miRNA binding site that decreases expression of BRCA1 indicates that the presence or absence of the SNP inhibits miRNA-mediated protection or increases miRNA-mediated repression of BRCA1 gene expression, thereby identifying a SNP that also increases a subject's risk of developing breast or ovarian cancer. In certain embodiments of this method, the test subject has been diagnosed with breast or ovarian cancer. In contrast, the control sample is obtained from a subject who has not been diagnosed with any cancer. Moreover the control sample can also be a control value retrieved from a database or clinical study. A world population is a geographical (European or African American) or ethnic population (Ashkenazi Jewish), the members of which for physical or cultural reasons would be expected to share similar genetic backgrounds.

With respect to methods of identifying SNPs, a miRNA binding site is determined empirically, identified in a database, or predicted using an algorithm. Moreover, the presence or absence of the SNP is determined empirically, identified in a database, or predicted using an algorithm.

Moreover, the invention provides a method of identifying a subject at risk of developing breast or ovarian cancer including: a) obtaining a DNA sample from a test subject; and b) determining the presence of at least one SNP selected from the group consisting of rs12516, rs8176318, rs3092995, and rs799912 in at least one DNA sequence from the sample, wherein the presence of the at least one SNP in the at least one DNA sequence increases the subject's risk of developing breast or ovarian cancer 10-fold compared to a normal subject. In a preferred embodiment, the method further includes the step of determining the presence of rs1060915, wherein the combined presence of rs1060915 and at least one SNP selected from the group consisting of rs12516, rs8176318, rs3092995, and rs799912 in the at least one DNA sequence increases the subject's risk of developing breast or ovarian cancer 100-fold compared to a normal subject. A normal subject is a subject who does not carry the common allele at rs12516, rs8176318, rs3092995, rs799912, or rs1060915.

The invention also provides a method of identifying a subject at risk of developing triple negative (TN) breast cancer comprising: a) obtaining a DNA sample from a test subject; and b) determining the presence of rs8176318 or rs1060915 in at least one DNA sequence from the sample, wherein the presence of rs8176318 or rs1060915 in the at least one DNA sequence increases the subject's risk of developing TN breast cancer compared to a normal subject. In a preferred embodiment, this method includes the step of determining the presence of rs8176318 and rs1060915, wherein the combined presence of rs8176318 and rs1060915 in the at least one DNA sequence further increases the subject's risk of developing TN breast cancer. A normal subject is a subject who does not carry rs8176318 or rs1060915. The test subject is preferably African American.

As described by the haplotypes, signature, and methods herein, breast cancer is sporadic or inherited. Moreover, ovarian cancer is sporadic or inherited.

### BRIEF DESCRIPTION OF THE DRAWINGS

FIG. 1 is a schematic representation of the biogenesis of miRNAs.

FIG. 2 is an annotation of a BRCA1 3′ UTR

FIG. 3 is a schematic comparison of the BRCA1 3′UTR in cancer populations. Findings are based on sequencing results from amplifying the whole BRCA1 3′UTR from 124 cancer DNA samples and 14 Yale control DNA samples.

FIG. 4 is a representation of BRCA1 3′ UTR genotyping at 3 SNP sites from 46 World populations, including 2,472 individuals.

FIG. 5 is a graphical representation of BRCA1 3′ UTR genotyping at 3 SNP sites from 7 cancer populations and 1 population of Yale controls, included in these 8 populations are 384 individuals.

FIG. 6 is a representation of 8 SNPs used to infer lineage and to accomplish haplotype analysis of the BRCA1 region of the genome. SNPs found within the BRCA1 gene include rs12516, rs8176318, rs3092995, rs1060915, and rs799912. SNPs surrounding BRCA1 include rs9911630, rs9908805, and rs17599948.

FIG. 7 is a representation of the proposed evolution of BRCA1 haplotypes. Ten most common haplotypes are shown here. Each haplotype can be explained by accumulation of variation on the ancestral haplotype (GGCCACTA, SEQ ID NO: 8). Most of the directly observed haplotypes can be ordered, differing by one derived nucleotide change. The two haplotypes that are boxed were unresolved regarding which occurred first in the lineage with the SNPs that were employed. The AGCCATTA (SEQ ID NO: 2) haplotype is currently the most commonly observed haplotype in the World. Two haplotypes, labeled “present everywhere”, are present in all regions of the World (GAACAGATA (SEQ ID NO: 17) and GAACGCTC (SEQ ID NO: 18)). The recombinant haplotype (AGCC-GCTG, SEQ ID NO: 19) is found in the new world only, indicating regions of South, Central and North America.

FIG. 8 is a representation of the BRCA1 Area Haplotype Data from 46 populations (2,472 individuals) around the World.

FIG. 9 is a representation of BRCA1 Area Haplotype Data for 7 Cancer Populations and 1 Yale control group (384 individuals). Population sizes: Control: 29, Breast/Ovarian: 17, Uterine: 55, Ovarian: 77, ER/PR+: 44, HER2+; 47, MP: 39, TN: 76.

FIG. 10 is a representation of the ethnicity breakdown of BRCA1.

FIG. 11 is a representation of the BRCA1 haplotype data coding region mutation status. 110 patients have been BRCA1 tested and analyzed by haplotype.

FIG. 12 is a schematic representation displaying BRCA1 area haplotype frequencies with TN and Yale Controls separated by Ethnicity data.

FIG. 13 is a schematic representation displaying BRCA1 area haplotype frequencies in TN breast cancer group separated by ethnicity and age.

FIG. 14 is a graph depicting allele frequency for the derived allele at each genotyped SNP (rs12516 allele A, rs8176318 allele A, and rs3092995 allele G) in each of the chosen populations. The SNPs were examined in 388 individuals: European American and African American controls, and breast cancer populations: TN, HER2+, and ER+/PR+ shown from left to right.

FIG. 15 is a graph depicting BRCA1 rare haplotype frequencies among breast cancer patients by age of diagnosis. All breast cancer patients with known age of diagnosis were evaluated for rare BRCA1 haplotype frequencies. Breast cancer patients were grouped as either less than or equal to 52 years of age or older than 52 years of age at time of diagnosis. The five rare haplotypes among controls but common in breast cancer patients are shown.

FIG. 16A is a graph depicting BRCA1 rare haplotype frequencies among breast cancer patients. Breast cancer patients were evaluated for haplotypes found to be rare among global control populations but common in breast cancer patients. The five rare haplotype frequencies are displayed along the Y-axis.

FIG. 16B is a schematic diagram depicting BRCA1 haplotype frequencies among breast cancer by ethnicity. European and African American breast cancer patients were evaluated for haplotype frequencies. European Americans and African Americans were added as controls. Nine common haplotypes are shown. Five additional haplotypes that are rare among controls but common in breast cancer patients are shown (these rare haplotypes are numbered, marked with an asterisk, and boxed). The remaining haplotype frequencies with non-zero estimates are combined into the residual class. The three 3′UTR polymorphisms are displayed in a bold font (occupying positions 2, 3, and 4 of the 8 nucleotide positions, if position 1 is the left-most nucleotide and position 8 is the right-most nucleotide) and the derived alleles within the 3′UTR are underlined.

FIG. 17A is a graph depicting BRCA1 rare haplotype frequencies among breast cancer patients by subtype. Breast cancer patients were grouped by subtype and evaluated for haplotypes found to be rare among global control populations but common in breast cancer patients. The five rare haplotype frequencies are displayed along the Y-axis.

FIG. 17B is a graph depicting rare haplotype frequencies by breast cancer subtype and ethnicity. European and African American breast cancer patients were further grouped by breast tumor subtype and evaluated for rare haplotype frequencies. European Americans and African Americans were added as controls. Five rare haplotypes among controls but common in breast cancer patients are shown.

FIGS. 18A-B are a pair of graphs depicting the transcriptional repression of a luciferase reporter construct following transfection of TN breast cancer cells (MDA MB 231 cells shown) with either wild type (WT, rs1060915G)) or mutant BRCA1 mRNA (BRCA1 gene containing the re1060915A variant allele) elements fused to a luciferase reporter. Luciferase reporters (25 ng) containing either the WT or variant BRCA1 mRNA elements were transfected into cells. Twenty-four hours post-transfection, transfected cells were lysed and assayed for dual luciferase activities. Variant allele (A) was normalized to the ancestral allele (G). Statistical significance determined by a students 2-tailed T-Test. Results indicated a 1.85 fold change in luciferase activity between WT and the variant BRCA1 element across all cell lines (9 cell lines tested). Thus, rs1060915A is a regulatory element within the BRCA1 gene. With rs1060915 present, miRNAs may not bind as efficaciously (as much or as tightly) or different miRNAs bind to BRCA1 allowing altered regulation of translation.

FIG. 19 is a schematic representation of the miRNAs that target a site surrounding rs1060915 within the BRCA1 gene. Four candidate miRNAs are predicted to bind to either the ancestral or variant allele of rs1060915, but not to an alternative SNP allele. Many others are predicted to bind with less dramatic interactions or changes.

BRCA1 rs1060915, positions 61-94
(SEQ ID NO: 28)
5′-AACAGCUACCCUUCCAUCAUAAGUGACUCUUCUG-3′.
Hsa-miR-7,
(SEQ ID NO: 29)
5′-UGGAAGACUAGUGAUUUUGUUGU-3′.
BRCA1 rs1060915, positions 79-105
(SEQ ID NO: 30)
5′-AUAAGUGACUCCUCUGCCCUUGAGGAC-3′.
Hsa-miR-129-5P,
(SEQ ID NO: 31)
5′-CUUUUUGCGGUCUGGGCUUGC-3′.
BRCA1 rs1060915, positions 45-93
(SEQ ID NO: 32)
5′-UGGGAGCCAGCCUUCUAACAGCUACCCUUCCAUCAUAAGUGACUCUUCU-3′.
Hsa-miR-185,
(SEQ ID NO: 33)
5′-UGGAGAGAAAGGCAGUUCCUGA-3′.
BRCA1 rs1060915, positions 44-96
(SEQ ID NO: 34)
5′-AUGGGAGCCAGCCUUCUAACAGCUACCCUUCCAUCAUAAGUGACUCUUCUGCC-3′.
Hsa-miR-298,
(SEQ ID NO: 35)
5′-AGCAGAAGCAGGGAGGUUCUCCCA-3′.

FIG. 20A is a graph depicting the significantly high levels of miR-7 expression in BRCA1 rare haplotype tumors compared to cancer patients without rare haplotypes (p=0.04). It is contemplated that miR-7 expression is correlated with the haplotype rather than the breast cancer subtype.

FIG. 20B is a graph depicting the frequency of miRNA expression as a function of miRNAs in TN breast cancer patients. MiR-7, miR-28, and miR-342 are highly expressed in BRCA1 tumors. For instance, miR-7 is highly expressed in TN breast cancer tumors. Although other breast cancer subtypes were not tested, it is contemplated that other subtypes in which rare BRCA1 haplotypes occur will also demonstrate high levels of miR-7 expression.

FIG. 21 is a graph depicting the binding efficacy of miR-7 on wild type (WT) BRCA1 (AA) and BRCA1 containing the rs1060915 SNP (GG). MiR-7 binding is altered in the presence of the rs1060915 SNP. HCC 1937+/+ cells transfected with ancestral or variant sequence (BRCA1 containing the rs1060915 SNP): (0.5 nM). MiR-7, but not the scrambled control, binds to the WT BRCA1 sequence, i.e. miR-7 specifically alters BRCA expression. Of note, altered expression is demonstrated by higher luciferase expression in this model. Neither miR-7 nor the scrambled control alters expression of the variant BRCA1, which was predicted; because there is no predicted binding site with the variant allele present (the variant allele destroys the miR-7 binding site that would otherwise be present in the WT BRCA1, and presumably protect BRCA1 and lead to higher levels of the mRNA or protein).

### DETAILED DESCRIPTION

Breast cancer is the most frequently diagnosed cancer and one of the leading causes of cancer death in women today. Clinical and molecular classification has successfully clustered breast cancer into subgroups and shown unique gene expression in categories that have prognostic significance. Among the categories emerging from these studies are estrogen receptor (ER) or progesterone receptor (PR) positive, HER2 receptor gene-amplified tumors, and triple negative ([TN] ER/PR/HER2-tumors). The ER/PR+ and HER2+ tumors together are most prevalent (80%), with basal-like or TN tumors accounting for approximately 15-20% of breast cancers (Irvin W J, Jr. and Carey L A. Eur J Cancer 2008; 44(18):2799-805). The TN phenotype represents an aggressive and poorly understood subclass of cancer that is most prevalent among younger women and in African American women.

BRCA1 coding sequence mutations are a well-known risk factor for breast cancer, however, these mutations account for less than 5% of all breast cancer cases yearly. Overall, breast tumors resulting from BRCA1 mutations are most frequently TN (57%) (Atchley D P, et al. J Clin Oncol 2008; 26(26):4282-8) or ER+ breast cancers (34%) (Tung N, et al. Breast Cancer Res; 12(1):R12.), and are rarely HER2+ breast cancers (about 3%) (Lakhani S R, et al. J Clin Oncol 2002; 20(9):2310-8.). TN tumors are often characterized by low expression of BRCA1 (Turner N, Tutt A, Ashworth A. Nature reviews 2004; 4(10):814-9), because BRCA1 mutations are quite rare. BRCA1 mutations only account for approximately 10-20% of the TN tumors (Young S R et al. BMC cancer 2009; 9:86; Malone K E, et al. Cancer research 2006; 66(16):8297-308; Nanda R, et al. JAMA 2005; 294(15):1925-33). These results suggest that there may be additional genetic factors associated with BRCA1 misexpression that could predispose individuals to breast cancer.

Haplotypes are patterns of several SNPs that are in linkage disequilibrium (LD) with one another within a gene or segment of DNA and are thus inherited as a unit. As haplotypes serve as markers for all measured and unmeasured alleles within a population, a study of haplotypes of a region of interest can narrow the search for causal SNPs. Previous studies of the association of BRCA1 haplotypes with breast cancer have yielded conflicting results. Cox et al., identified five common haplotypes (>5%) that could be predicted by four tagging SNPs. Testing of these SNPs showed that one of the haplotypes predicted a 20% increased risk (odds ratio 1.18, 95% confidence interval 1.02-1.37) of sporadic breast cancer in Caucasian women in the Nurses' Health Study (Cox D G, et al. Breast Cancer Res 2005; 7(2):R171-5). There was significant interaction (p=0.05) between this haplotype, positive family history and breast cancer risk (Cox D G, et al. Breast Cancer Res 2005; 7(2):R171-5). In contrast, Freedman et al. tested common variation across the BRCA1 locus in a cohort from the Multiethnic Cohort Study. This group was not able to show that common variants in BRCA1 substantially influence sporadic breast cancer risk (Freedman M L, et al. Cancer research 2005; 65(16):7516-22). These haplotype studies focused primarily on variation at SNPs in the coding and intronic regions of BRCA1 (Dunning A M, et al. Human molecular genetics 1997; 6(2):285-9; Bau D T, et al. Cancer research 2004; 64(14):5013-9).

MiRNAs are a class of 22-nucleotide non-coding RNAs that are evolutionarily-conserved and are aberrantly expressed in virtually all cancers, where they function as a novel class of oncogenes or tumor suppressors. The ability of miRNAs to bind to messenger (mRNA) in the 3′UTR is critical for regulating mRNA level and protein expression, binding which can be affected by single nucleotide polymorphisms. Recent data indicates that variants in the 3′UTR of cancer genes are strong genetic markers of cancer risk (Chin L J, et al. Cancer research 2008; 68(20):8535-40; Landi D, et al. Carcinogenesis 2008; 29(3):579-84; Pongsavee M, et al. Genetic testing and molecular biomarkers 2009; 13(3):307-17).

The BRCA1 3′ UTR has been recently studied for such miRNA-binding site SNPs and the derived (and less frequent) alleles at rs12516 and rs8176318 showed a positive association with familial breast and ovarian cancer in Thai women. The study found that homozygosity for the derived alleles, A, at both SNP sites are found in cancer patients at triple the frequency as seen in unaffected Thais, yielding a significant cancer association (p=0.007). Functional analysis showed reduced activity of BRCA1 function with the derived alleles at both sites when present on the same chromosome, i.e. in cis, with the greatest reduction seen with the derived allele at rs8176318 (Pongsavee M, et al. Genetic testing and molecular biomarkers 2009; 13(3):307-17). This study additionally found that the 3′UTR variants were not associated with known BRCA1 mutations. In addition, a study in 1998 reported an allele at a third SNP in the BRCA1 3′UTR, rs3092995, as being associated with increased risk of breast cancer in African American women. The rarer, derived G allele was found to be more common in African American breast cancer cases than African American controls. The age-adjusted OR for breast cancer among African American women and the G allele was 3.5 (95% CI, 1.2-10) (Newman B, et al. JAMA 1998; 279(12):915-21).

The invention is based in part on the understanding that studying haplotypes that include functional 3′UTR variants should better identify BRCA1 haplotypes associated with breast cancer risk. Furthermore, because BRCA1 dysfunction varies by breast cancer subtype, these haplotypes were evaluated by breast cancer subtype. Consequently, 3′UTR SNPs were identified in breast cancer patients, one of which was individually significant. Subsequently, haplotype analysis was performed with these variants and five SNPs surrounding the BRCA1 3′UTR to determine association of haplotypes with breast cancer. This study further identified five haplotypes commonly shared in breast cancer patients but rare in non-cancerous populations. These rare BRCA1 haplotypes represent new genetic markers of BRCA1 dysfunction associated with breast cancer risk.

Cancer is a multifaceted disease caused by uncontrolled cellular proliferation and the survival of damaged cells, which results in tumor formation. Cells have developed several safeguards to ensure that cell division, differentiation, and death occur properly throughout life. Many regulatory factors switch on or off genes that guide cellular proliferation and differentiation (Esquela-Kerscher, A. & Slack, F. J. Nat Rev Cancer, 2006, 6: 259-69). Damage to these tumor-suppressor genes and oncogenes, is selected for in cancer. Most tumor-suppressor genes and oncogenes are first transcribed and then translated into protein to express their affects. Recent data indicates that small non-protein-coding RNA molecules, called MicroRNAs (miRNAs), also can function as either tumor suppressors or oncogenes (Medina, P. P. and Slack, F. J. Cell Cycle 2008. 7, 2485-92). Among human diseases, it has been shown that miRNAs are aberrantly expressed or mutated in cancer, suggesting that they play a role as a novel class of oncogenes or tumor suppressor genes more accurately referred to as oncomirs (Iorio, M. V. et al. Cancer Res 2005. 65, 7065-70).

MiRNAs are evolutionarily conserved, short, non-protein-coding, single-stranded RNAs that represent a novel class of posttranscriptional gene regulators. Studies have shown differential miRNA expression profiles between tumors and normal tissue (Medina, P. P. and Slack, F. J. Cell Cycle 2008. 7, 2485-92), and miRNAs are at abnormal levels in virtually all cancer subtypes studied (Esquela-Kerscher, A. & Slack, F. J. Nat Rev Cancer 2006. 6, 259-69). MiRNAs bind to the 3′ untranslated regions (UTRs) of their target genes and each regulate hundreds of different target transcripts, which implies that miRNAs may be able to regulate up to 30% of the protein-coding genes in the human genome (Chen, K. et al. Carcinogenesis 2008. 29, 1306-11). Therefore, the effects of a malfunctioning miRNA would likely be pleotropic, and their aberrant expression could potentially unbalance the cell's homeostasis, contributing to diseases, including cancer.

The ability of the miRNA to bind to the messenger RNA (mRNA) is critical for regulating mRNA level and protein expression. However, this binding can be affected by single nucleotide polymorphisms (SNPs) that can reside in the miRNA target site, which can either eliminate existing binding sites or create erroneous binding sites (Chen, K. et al. Carcinogenesis 2008. 29, 1306-11). The role of miRNA target site SNPs in diseases, including cancer, is just beginning to be defined.

### MiRNAs

MiRNAs are a broad class of small non-protein-coding RNA molecules of approximately 22 nucleotides in length that function in posttranscriptional gene regulation by pairing to the mRNA of protein-coding genes. Recently, it has been shown that miRNAs play roles at human cancer loci with evidence that they regulate proteins known to be critical in survival pathways (Esquela-Kerscher, A. & Slack, F. J. Nat Rev Cancer 2006, 6: 259-69; Ambros, V. Cell 2001, 107: 823-6; Slack, F. J. and Weidhaas, J. B. Future Oncol 2006, 2: 73-82). Because miRNAs control many downstream targets, it is possible for them to act as novel targets for the treatment in cancer.

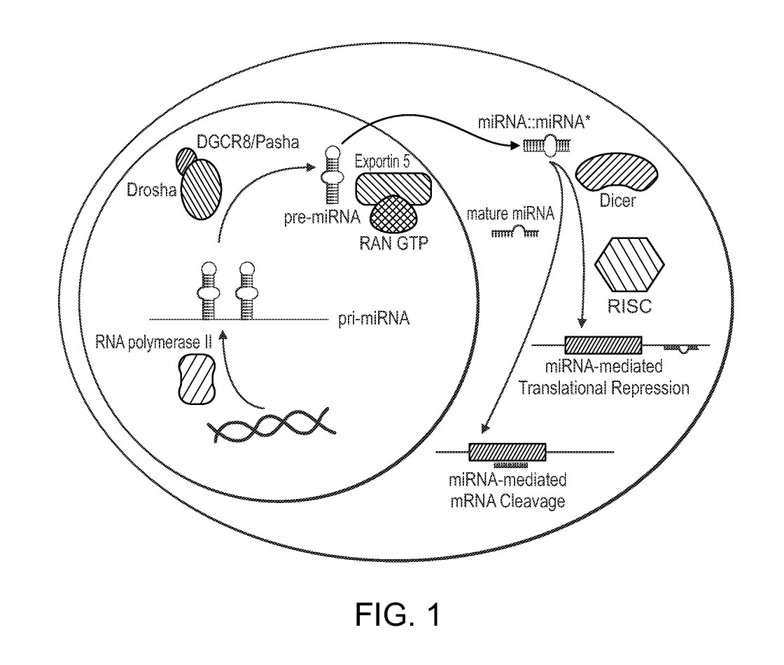

The basic synthesis and maturation of miRNAs can be visualized in FIG. 1 (Esquela-Kerscher, A. and Slack, F. J. Nat Rev Cancer 2006. 6, 259-69). In brief, miRNAs are transcribed from miRNA genes by RNA Polymerase II in the nucleus to form long primary RNAs (pri-miRNA) transcripts, which are capped and polyadenylated (Esquela-Kerscher, A. and Slack, F. J. Nat Rev Cancer 2006. 6, 259-69; Lee, Y. et al. Embo J 2002. 21, 4663-70). These pri-miRNAs can be several kilobases long, and are processed in the nucleus by the RNAaseIII enzyme Drosha and its cofactor, Pasha, to release the approximately 70-nucleotide stem-loop structured miRNA precursor (pre-miRNA). Pre-miRNAs are exported from the nucleus to the cytoplasm by exportin 5 in a Ran-guanosine triphosphate (GTP)-dependent manner, where they are then processed by Dicer, an RNase III enzyme. This causes the release of an approximately 22-base nucleotide, double-stranded, miRNA:miRNA duplex that is incorporated into a RNA-induced silencing complex (miRISC). At this point the complex is now capable of regulating its target genes.

FIG. 1 depicts how gene expression regulation can occur in one of two ways that depends on the degree of complimentarity between the miRNA and its target. MiRNAs that bind to mRNA targets with imperfect complimentarity block target gene expression at the level of protein translation. Complimentary sites for miRNAs using this mechanism are generally found in the 3′ UTR of the target mRNA genes. MiRNAs that bind to their mRNA targets with perfect complimentarity induce target-mRNA cleavage. MiRNAs using this mechanism bind to miRNA complimentary sites that are generally found in the coding sequence or open reading frame (ORF) of the mRNA target.

In mammals, miRNAs are gene regulators that are found at abnormal levels in virtually all cancer subtypes studied. Proper miRNA binding to their target genes is critical for regulating the mRNA level and protein expression. However, successful binding can be affected by polymorphisms that can reside in the miRNA binding sites, which can either abolish existing binding sites or create illegitimate binding sites. Therefore, polymorphisms in miRNA binding sites can have a wide-range of effects on gene and protein expression and represent another source of genetic variability that can influence the risk of human diseases, including cancer. The role of miRNA binding site SNPs in disease is just beginning to be defined and the identification of SNPs in breast cancer genes that modify the ability of miRNAs to bind, thereby affecting target gene regulation and risk of breast and/or ovarian cancer may help identify novel approaches for recognizing patients with increased breast and/or ovarian cancer risk.

MiRNAs not only target noncoding regions of target mRNAs and genes, but also protein coding regions. The mechanisms of miRNA:target recognition may differ between noncoding and coding regions. When a miRNA recognizes a binding site within a protein coding region, the transcriptional silencing effect of miRNA binding may be decreased compared to the result of miRNA recognition and binding in a noncoding region. Moreover, miRNA binding site seed regions located within protein coding regions may require a greater number of nucleotides bound to the miRNAs than seed regions of binding sites located in noncoding regions. A SNP may also occur in a miRNA binding site located within a coding region, and, consequently, affect the ability of one or more miRNA(s) to regulate the expression of the target gene.

It is contemplated that a SNP that occurs in a coding region and which affects the activity of a miRNA could have a quantitatively or qualitatively similar effect on the expression of the target protein. Alternatively, a SNP that occurs in a coding region and which affects the activity of a miRNA could have a quantitatively or qualitatively different effect on the expression of the target protein. It is further contemplated that when a SNP is simultaneously present in a noncoding and a coding region, and these SNPs both affect the binding of one or more miRNAs to bind to their respective binding sites that these individual SNPs act synergistically to affect expression of the target transcript or protein.

MiRNA activity is further influenced by the cell cycle. During cell cycle arrest, certain miRNAs have been shown to activate translation or induce up-regulation of target mRNAs (Vasudevan S. et al. Science, 2007. 318(5858):1931-4). Thus, the activity of miRNAs may oscillate between transcriptional repression during, for instance, the growth (G₁ and G₂) and synthesis (S₁) phases, of the cell cycle and transcriptional activation during the cell cycle arrest (G₀). While not wishing to be bound by theory, cancer cells enter and complete the cell cycle at inappropriate times or with inappropriate frequency. Moreover, cancer cells often complete the cell cycle without the safeguards of functioning or adequate levels of DNA repair proteins, including BRCA1. Whereas a healthy, noncancerous, cell may be in the G₀ phase, in which a miRNA bound to BRCA1 upregulates expression of the tumor suppressor protein, a cancer cell is most frequently in a growth phase, during which miRNAs transcriptionally repress protein expression. The invention contemplates that the presence of a SNP in a noncoding and/or coding region that affects the activity or binding of at least one miRNA may prevent upregulation of BRCA1 for instance, and this may induce a healthy cell to enter the cell cycle, during which additional miRNAs further repress the expression of BRCA1 and/or other tumor suppressor genes.

### Single Nucleotide Polymorphisms (SNPs)

A single nucleotide polymorphism (SNP) is a DNA sequence variation occurring when a single nucleotide in the genome (or other shared sequence) differs between members of a species (or between paired chromosomes in an individual). SNPs may fall within coding sequences of genes, non-coding regions of genes, or in the intergenic regions between genes. SNPs within a coding sequence will not necessarily change the amino acid sequence of the protein that is produced, due to degeneracy of the genetic code. A SNP mutation that results in a new DNA sequence that encodes the same polypeptide sequence is termed synonymous (also referred to as a silent mutation). Conversely, a SNP mutation that results in a new DNA sequence that encodes a different polypeptide sequence is termed non-synonymous. SNPs that are not in protein-coding regions may still have consequences for gene splicing, transcription factor binding, or the sequence of non-coding RNA.

For the methods of the invention, SNPs occurring within non-coding RNA regions are particularly important because those regions contain regulatory sequences which are complementary to miRNA molecules and required for interaction with other regulatory factors. SNPs occurring within genomic sequences are transcribed into mRNA transcripts which are targeted by miRNA molecules for degradation or translational silencing. SNPs occurring within the 3′ untranslated region (UTR) of the genomic sequence or mRNA of a gene are of particular importance to the methods of the invention.

### BRCA1

BRCA1 (BReast CAncer 1, early onset) is a human tumor suppressor gene. Although BRCA1 is most commonly associated with breast cancer, the BRCA1 gene is present in every cell of the body. As a tumor suppressor gene, BRCA1 negatively regulates cell proliferation and prevents mutations from being introduced by either repairing damaged DNA or initiating cellular suicide programs for those cells whose DNA is too damaged to repair.

If a tumor suppressor gene like BRCA1 is mutated or misregulated, then its function is inhibited, and the cell may proceed through proliferation with imperfectly replicated DNA. Moreover, the cell may enter the cell cycle too frequently. In these circumstances, a tumor forms. A cancerous tumor, as opposed to a benign tumor, demonstrates uncontrolled growth, invasion and destruction of adjacent tissues, and metastasis to other locations in the body via lymph or blood.

Specifically, BRCA1 repairs double-strand breaks in DNA by homologous recombination, a process by which homologous intact nucleotide sequences are exchanged between two similar or identical strands of DNA, e.g. sequences from a sister chromatid, homologous chromosome, or from the same chromosome (depending on cell cycle phase) as a template. However, the BRCA1 protein does not function alone. BRCA1 combines with other tumor suppressor proteins, DNA damage sensors, and signal transducers to form a large multi-subunit protein complex known as the BRCA1-associated genome surveillance complex (BASC).

Despite the fact that the BRCA1 protein can form a complex to carry out cellular functions, mutations in the BRCA1 gene are sufficient to deregulate cell repair and proliferation programs. Importantly, the invention provides single nucleotide polymorphisms (SNPs), haplotypes, methods for identifying SNPs that prevent or inhibit the function of one or more miRNAs from binding to a coding or non-coding region of the BRCA1 gene, and methods for predicting the increased risk of developing cancer by detecting at least one polymorphism described herein.

The invention provides methods for identifying and characterizing SNPs within BRCA1. While not wishing to be bound by theory, it is contemplated that the SNPs disclosed herein, and those identified using the methods disclosed herein, which occur within miRNA binding sites, or otherwise affect miRNA activity, cause “tighter” miRNA interactions or binding between one or more miRNAs and BRCA1, or in some cases “looser” miRNA interactions or loss of these interactions. The increased binding efficacy or activity of these miRNAs in the 3′UTR leads to decreased transcription of BRCA1, and overall, lower levels of BRCA1 protein in the cell. The possible loss of binding within an exon might also lead to lower levels of BRCA1. Therefore, the SNPs identified herein repress the BRCA1 tumor suppressor gene, allowing cell repair and proliferation mechanisms to proceed without the supervision of BRCA1. As described above, unregulated cell proliferation results in an increased risk of developing cancer.

Exemplary BRCA1 genes and transcripts are provided below. All GenBank records (provided by NCBI Accession No.) are herein incorporated by reference.

```
Human BRCA1, transcript variant 1, is encoded by the nucleic acid sequence of
NCBI Accession No. NM_007294 and SEQ ID NO: 11).
      1 gtaccttgat ttcgtattct gagaggctgc tgcttagcgg tagccccttg gtttccgtgg
    61 caacggaaaa gcgcgggaat tacagataaa ttaaaactgc gactgcgcgg cgtgagctcg
  121 ctgagacttc ctggacgggg gacaggctgt ggggtttctc agataactgg gcccctgcgc
  181 tcaggaggcc ttcaccctct gctctgggta aagttcattg gaacagaaag aaatggattt
  241 atctgctctt cgcgttgaag aagtacaaaa tgtcattaat gctatgcaga aaatcttaga
  301 gtgtcccatc tgtctggagt tgatcaagga acctgtctcc acaaagtgtg accacatatt
  361 ttgcaaattt tgcatgctga aacttctcaa ccagaagaaa gggccttcac agtgtccttt
  421 atgtaagaat gatataacca aaaggagcct acaagaaagt acgagattta gtcaacttgt
  481 tgaagagcta ttgaaaatca tttgtgcttt tcagcttgac acaggtttgg agtatgcaaa
  541 cagctataat tttgcaaaaa aggaaaataa ctctcctgaa catctaaaag atgaagtttc
  601 tatcatccaa agtatgggct acagaaaccg tgccaaaaga cttctacaga gtgaacccga
  661 aaatccttcc ttgcaggaaa ccagtctcag tgtccaactc tctaaccttg gaactgtgag
  721 aactctgagg acaaagcagc ggatacaacc tcaaaagacg tctgtctaca ttgaattggg
  781 atctgattct tctgaagata ccgttaataa ggcaacttat tgcagtgtgg gagatcaaga
  841 attgttacaa atcacccctc aaggaaccag ggatgaaatc agtttggatt ctgcaaaaaa
  901 ggctgcttgt gaattttctg agacggatgt aacaaatact gaacatcatc aacccagtaa
  961 taatgatttg aacaccactg agaagcgtgc agctgagagg catccagaaa agtatcaggg
1021 tagttctgtt tcaaacttgc atgtggagcc atgtggcaca aatactcatg ccagctcatt
1081 acagcatgag aacagcagtt tattactcac taaagacaga atgaatgtag aaaaggctga
1141 attctgtaat aaaagcaaac agcctggctt agcaaggagc caacataaca gatgggctgg
1201 aagtaaggaa acatgtaatg ataggcggac tcccagcaca gaaaaaaagg tagatctgaa
1261 tgctgatccc ctgtgtgaga gaaaagaatg gaataagcag aaactgccat gctcagagaa
1321 tcctagagat actgaagatg ttccttggat aacactaaat agcagcattc agaaagttaa
1381 tgagtggttt tccagaagtg atgaactgtt aggttctgat gactcacatg atggggagtc
1441 tgaatcaaat gccaaagtag ctgatgtatt ggacgttcta aatgaggtag atgaatattc
1501 tggttcttca gagaaaatag acttactggc cagtgatcct catgaggctt taatatgtaa
1561 aagtgaaaga gttcactcca aatcagtaga gagtaatatt gaagacaaaa tatttgggaa
1621 aacctatcgg aagaaggcaa gcctccccaa cttaagccat gtaactgaaa atctaattat
1681 aggagcattt gttactgagc cacagataat acaagagcgt cccctcacaa ataaattaaa
1741 gcgtaaaagg agacctacat caggccttca tcctgaggat tttatcaaga aagcagattt
1801 ggcagttcaa aagactcctg aaatgataaa tcagggaact aaccaaacgg agcagaatgg
1861 tcaagtgatg aatattacta atagtggtca tgagaataaa acaaaaggtg attctattca
1921 gaatgagaaa aatcctaacc caatagaatc actcgaaaaa gaatctgctt tcaaaacgaa
1981 agctgaacct ataagcagca gtataagcaa tatggaactc gaattaaata tccacaattc
2041 aaaagcacct aaaaagaata ggctgaggag gaagtcttct accaggcata ttcatgcgct
2101 tgaactagta gtcagtagaa atctaagccc acctaattgt actgaattgc aaattgatag
2161 ttgttctagc agtgaagaga taaagaaaaa aaagtacaac caaatgccag tcaggcacag
2221 cagaaaccta caactcatgg aaggtaaaga acctgcaact ggagccaaga agagtaacaa
2281 gccaaatgaa cagacaagta aaagacatga cagcgatact ttcccagagc tgaagttaac
2341 aaatgcacct ggttctttta ctaagtgttc aaataccagt gaacttaaag aatttgtcaa
2401 tcctagcctt ccaagagaag aaaaagaaga gaaactagaa acagttaaag tgtctaataa
2461 tgctgaagac cccaaagatc tcatgttaag tggagaaagg gttttgcaaa ctgaaagatc
2521 tgtagagagt agcagtattt cattggtacc tggtactgat tatggcactc aggaaagtat
2581 ctcgttactg gaagttagca ctctagggaa ggcaaaaaca gaaccaaata aatgtgtgag
2641 tcagtgtgca gcatttgaaa accccaaggg actaattcat ggttgttcca aagataatag
2701 aaatgacaca gaaggcttta agtatccatt gggacatgaa gttaaccaca gtcgggaaac
2761 aagcatagaa atggaagaaa gtgaacttga tgctcagtat ttgcagaata cattcaaggt
2821 ttcaaagcgc cagtcatttg ctccgttttc aaatccagga aatgcagaag aggaatgtgc
2881 aacattctct gcccactctg ggtccttaaa gaaacaaagt ccaaaagtca cttttgaatg
2941 tgaacaaaag gaagaaaatc aaggaaagaa tgagtctaat atcaagcctg tacagacagt
3001 taatatcact gcaggctttc ctgtggttgg tcagaaagat aagccagttg ataatgccaa
3061 atgtagtatc aaaggaggct ctaggttttg tctatcatct cagttcagag gcaacgaaac
3121 tggactcatt actccaaata aacatggact tttacaaaac ccatatcgta taccaccact
3181 ttttcccatc aagtcatttg ttaaaactaa atgtaagaaa aatctgctag aggaaaactt
3241 tgaggaacat tcaatgtcac ctgaaagaga aatgggaaat gagaacattc caagtacagt
3301 gagcacaatt agccgtaata acattagaga aaatgttttt aaagaagcca gctcaagcaa
3361 tattaatgaa gtaggttcca gtactaatga agtgggctcc agtattaatg aaataggttc
3421 cagtgatgaa aacattcaag cagaactagg tagaaacaga gggccaaaat tgaatgctat
3481 gcttagatta ggggttttgc aacctgaggt ctataaacaa agtcttcctg gaagtaattg
3541 taagcatcct gaaataaaaa agcaagaata tgaagaagta gttcagactg ttaatacaga
3601 tttctctcca tatctgattt cagataactt agaacagcct atgggaagta gtcatgcatc
3661 tcaggtttgt tctgagacac ctgatgacct gttagatgat ggtgaaataa aggaagatac
3721 tagttttgct gaaaatgaca ttaaggaaag ttctgctgtt tttagcaaaa gcgtccagaa
3781 aggagagctt agcaggagtc ctagcccttt cacccataca catttggctc agggttaccg
3841 aagaggggcc aagaaattag agtcctcaga agagaactta tctagtgagg atgaagagct
3901 tccctgcttc caacacttgt tatttggtaa agtaaacaat ataccttctc agtctactag
3961 gcatagcacc gttgctaccg agtgtctgtc taagaacaca gaggagaatt tattatcatt
4021 gaagaatagc ttaaatgact gcagtaacca ggtaatattg gcaaaggcat ctcaggaaca
4081 tcaccttagt gaggaaacaa aatgttctgc tagcttgttt tcttcacagt gcagtgaatt
4141 ggaagacttg actgcaaata caaacaccca ggatcctttc ttgattggtt cttccaaaca
4201 aatgaggcat cagtctgaaa gccagggagt tggtctgagt gacaaggaat tggtttcaga
4261 tgatgaagaa agaggaacgg gcttggaaga aaataatcaa gaagagcaaa gcatggattc
4321 aaacttaggt gaagcagcat ctgggtgtga gagtgaaaca agcgtctctg aagactgctc
4381 agggctatcc tctcagagtg acattttaac cactcagcag agggatacca tgcaacataa
4441 cctgataaag ctccagcagg aaatggctga actagaagct gtgttagaac agcatgggag
4501 ccagccttct aacagctacc cttccatcat aagtgactct tctgcccttg aggacctgcg
4561 aaatccagaa caaagcacat cagaaaaagc agtattaact tcacagaaaa gtagtgaata
4621 ccctataagc cagaatccag aaggcctttc tgctgacaag tttgaggtgt ctgcagatag
4681 ttctaccagt aaaaataaag aaccaggagt ggaaaggtca tccccttcta aatgcccatc
4741 attagatgat aggtggtaca tgcacagttg ctctgggagt cttcagaata gaaactaccc
4801 atctcaagag gagctcatta aggttgttga tgtggaggag caacagctgg aagagtctgg
4861 gccacacgat ttgacggaaa catcttactt gccaaggcaa gatctagagg gaacccctta
4921 cctggaatct ggaatcagcc tcttctctga tgaccctgaa tctgatcctt ctgaagacag
4981 agccccagag tcagctcgtg ttggcaacat accatcttca acctctgcat tgaaagttcc
5041 ccaattgaaa gttgcagaat ctgcccagag tccagctgct gctcatacta ctgatactgc
5101 tgggtataat gcaatggaag aaagtgtgag cagggagaag ccagaattga cagcttcaac
5161 agaaagggtc aacaaaagaa tgtccatggt ggtgtctggc ctgaccccag aagaatttat
5221 gctcgtgtac aagtttgcca gaaaacacca catcacttta actaatctaa ttactgaaga
5281 gactactcat gttgttatga aaacagatgc tgagtttgtg tgtgaacgga cactgaaata
5341 ttttctagga attgcgggag gaaaatgggt agttagctat ttctgggtga cccagtctat
5401 taaagaaaga aaaatgctga atgagcatga ttttgaagtc agaggagatg tggtcaatgg
5461 aagaaaccac caaggtccaa agcgagcaag agaatcccag gacagaaaga tcttcagggg
5521 gctagaaatc tgttgctatg ggcccttcac caacatgccc acagatcaac tggaatggat
5581 ggtacagctg tgtggtgctt ctgtggtgaa ggagctttca tcattcaccc ttggcacagg
5641 tgtccaccca attgtggttg tgcagccaga tgcctggaca gaggacaatg gcttccatgc
5701 aattgggcag atgtgtgagg cacctgtggt gacccgagag tgggtgttgg acagtgtagc
5761 actctaccag tgccaggagc tggacaccta cctgataccc cagatccccc acagccacta
5821 ctgactgcag ccagccacag gtacagagcc acaggacccc aagaatgagc ttacaaagtg
5881 gcctttccag gccctgggag ctcctctcac tcttcagtcc ttctactgtc ctggctacta
5941 aatattttat gtacatcagc ctgaaaagga cttctggcta tgcaagggtc ccttaaagat
6001 tttctgcttg aagtctccct tggaaatctg ccatgagcac aaaattatgg taatttttca
6061 cctgagaaga ttttaaaacc atttaaacgc caccaattga gcaagatgct gattcattat
6121 ttatcagccc tattctttct attcaggctg ttgttggctt agggctggaa gcacagagtg
6181 gcttggcctc aagagaatag ctggtttccc taagtttact tctctaaaac cctgtgttca
6241 caaaggcaga gagtcagacc cttcaatgga aggagagtgc ttgggatcga ttatgtgact
6301 taaagtcaga atagtccttg ggcagttctc aaatgttgga gtggaacatt ggggaggaaa
6361 ttctgaggca ggtattagaa atgaaaagga aacttgaaac ctgggcatgg tggctcacgc
6421 ctgtaatccc agcactttgg gaggccaagg tgggcagatc actggaggtc aggagttcga
6481 aaccagcctg gccaacatgg tgaaacccca tctctactaa aaatacagaa attagccggt
6541 catggtggtg gacacctgta atcccagcta ctcaggtggc taaggcagga gaatcacttc
6601 agcccgggag gtggaggttg cagtgagcca agatcatacc acggcactcc agcctgggtg
6661 acagtgagac tgtggctcaa aaaaaaaaaa aaaaaaagga aaatgaaact agaagagatt
6721 tctaaaagtc tgagatatat ttgctagatt tctaaagaat gtgttctaaa acagcagaag
6781 attttcaaga accggtttcc aaagacagtc ttctaattcc tcattagtaa taagtaaaat
6841 gtttattgtt gtagctctgg tatataatcc attcctctta aaatataaga cctctggcat
6901 gaatatttca tatctataaa atgacagatc ccaccaggaa ggaagctgtt gctttctttg
6961 aggtgatttt tttcctttgc tccctgttgc tgaaaccata cagcttcata aataattttg
7021 cttgctgaag gaagaaaaag tgtttttcat aaacccatta tccaggactg tttatagctg
7081 ttggaaggac taggtcttcc ctagcccccc cagtgtgcaa gggcagtgaa gacttgattg
7141 tacaaaatac gttttgtaaa tgttgtgctg ttaacactgc aaataaactt ggtagcaaac
7201 acttccaaaa aaaaaaaaaa aaaa
Human BRCA1, transcript variant 2, is encoded by nucleic acid sequence of NCBI
Accession No. NM_007300 and SEQ ID NO: 12).
      1 gtaccttgat ttcgtattct gagaggctgc tgcttagcgg tagccccttg gtttccgtgg
    61 caacggaaaa gcgcgggaat tacagataaa ttaaaactgc gactgcgcgg cgtgagctcg
  121 ctgagacttc ctggacgggg gacaggctgt ggggtttctc agataactgg gcccctgcgc
  181 tcaggaggcc ttcaccctct gctctgggta aagttcattg gaacagaaag aaatggattt
  241 atctgctctt cgcgttgaag aagtacaaaa tgtcattaat gctatgcaga aaatcttaga
  301 gtgtcccatc tgtctggagt tgatcaagga acctgtctcc acaaagtgtg accacatatt
  361 ttgcaaattt tgcatgctga aacttctcaa ccagaagaaa gggccttcac agtgtccttt
  421 atgtaagaat gatataacca aaaggagcct acaagaaagt acgagattta gtcaacttgt
  481 tgaagagcta ttgaaaatca tttgtgcttt tcagcttgac acaggtttgg agtatgcaaa
  541 cagctataat tttgcaaaaa aggaaaataa ctctcctgaa catctaaaag atgaagtttc
  601 tatcatccaa agtatgggct acagaaaccg tgccaaaaga cttctacaga gtgaacccga
  661 aaatccttcc ttgcaggaaa ccagtctcag tgtccaactc tctaaccttg gaactgtgag
  721 aactctgagg acaaagcagc ggatacaacc tcaaaagacg tctgtctaca ttgaattggg
  781 atctgattct tctgaagata ccgttaataa ggcaacttat tgcagtgtgg gagatcaaga
  841 attgttacaa atcacccctc aaggaaccag ggatgaaatc agtttggatt ctgcaaaaaa
  901 ggctgcttgt gaattttctg agacggatgt aacaaatact gaacatcatc aacccagtaa
  961 taatgatttg aacaccactg agaagcgtgc agctgagagg catccagaaa agtatcaggg
1021 tagttctgtt tcaaacttgc atgtggagcc atgtggcaca aatactcatg ccagctcatt
1081 acagcatgag aacagcagtt tattactcac taaagacaga atgaatgtag aaaaggctga
1141 attctgtaat aaaagcaaac agcctggctt agcaaggagc caacataaca gatgggctgg
1201 aagtaaggaa acatgtaatg ataggcggac tcccagcaca gaaaaaaagg tagatctgaa
1261 tgctgatccc ctgtgtgaga gaaaagaatg gaataagcag aaactgccat gctcagagaa
1321 tcctagagat actgaagatg ttccttggat aacactaaat agcagcattc agaaagttaa
1381 tgagtggttt tccagaagtg atgaactgtt aggttctgat gactcacatg atggggagtc
1441 tgaatcaaat gccaaagtag ctgatgtatt ggacgttcta aatgaggtag atgaatattc
1501 tggttcttca gagaaaatag acttactggc cagtgatcct catgaggctt taatatgtaa
1561 aagtgaaaga gttcactcca aatcagtaga gagtaatatt gaagacaaaa tatttgggaa
1621 aacctatcgg aagaaggcaa gcctccccaa cttaagccat gtaactgaaa atctaattat
1681 aggagcattt gttactgagc cacagataat acaagagcgt cccctcacaa ataaattaaa
1741 gcgtaaaagg agacctacat caggccttca tcctgaggat tttatcaaga aagcagattt
1801 ggcagttcaa aagactcctg aaatgataaa tcagggaact aaccaaacgg agcagaatgg
1861 tcaagtgatg aatattacta atagtggtca tgagaataaa acaaaaggtg attctattca
1921 gaatgagaaa aatcctaacc caatagaatc actcgaaaaa gaatctgctt tcaaaacgaa
1981 agctgaacct ataagcagca gtataagcaa tatggaactc gaattaaata tccacaattc
2041 aaaagcacct aaaaagaata ggctgaggag gaagtcttct accaggcata ttcatgcgct
2101 tgaactagta gtcagtagaa atctaagccc acctaattgt actgaattgc aaattgatag
2161 ttgttctagc agtgaagaga taaagaaaaa aaagtacaac caaatgccag tcaggcacag
2221 cagaaaccta caactcatgg aaggtaaaga acctgcaact ggagccaaga agagtaacaa
2281 gccaaatgaa cagacaagta aaagacatga cagcgatact ttcccagagc tgaagttaac
2341 aaatgcacct ggttctttta ctaagtgttc aaataccagt gaacttaaag aatttgtcaa
2401 tcctagcctt ccaagagaag aaaaagaaga gaaactagaa acagttaaag tgtctaataa
2461 tgctgaagac cccaaagatc tcatgttaag tggagaaagg gttttgcaaa ctgaaagatc
2521 tgtagagagt agcagtattt cattggtacc tggtactgat tatggcactc aggaaagtat
2581 ctcgttactg gaagttagca ctctagggaa ggcaaaaaca gaaccaaata aatgtgtgag
2641 tcagtgtgca gcatttgaaa accccaaggg actaattcat ggttgttcca aagataatag
2701 aaatgacaca gaaggcttta agtatccatt gggacatgaa gttaaccaca gtcgggaaac
2761 aagcatagaa atggaagaaa gtgaacttga tgctcagtat ttgcagaata cattcaaggt
2821 ttcaaagcgc cagtcatttg ctccgttttc aaatccagga aatgcagaag aggaatgtgc
2881 aacattctct gcccactctg ggtccttaaa gaaacaaagt ccaaaagtca cttttgaatg
2941 tgaacaaaag gaagaaaatc aaggaaagaa tgagtctaat atcaagcctg tacagacagt
3001 taatatcact gcaggctttc ctgtggttgg tcagaaagat aagccagttg ataatgccaa
3061 atgtagtatc aaaggaggct ctaggttttg tctatcatct cagttcagag gcaacgaaac
3121 tggactcatt actccaaata aacatggact tttacaaaac ccatatcgta taccaccact
3181 ttttcccatc aagtcatttg ttaaaactaa atgtaagaaa aatctgctag aggaaaactt
3241 tgaggaacat tcaatgtcac ctgaaagaga aatgggaaat gagaacattc caagtacagt
3301 gagcacaatt agccgtaata acattagaga aaatgttttt aaagaagcca gctcaagcaa
3361 tattaatgaa gtaggttcca gtactaatga agtgggctcc agtattaatg aaataggttc
3421 cagtgatgaa aacattcaag cagaactagg tagaaacaga gggccaaaat tgaatgctat
3481 gcttagatta ggggttttgc aacctgaggt ctataaacaa agtcttcctg gaagtaattg
3541 taagcatcct gaaataaaaa agcaagaata tgaagaagta gttcagactg ttaatacaga
3601 tttctctcca tatctgattt cagataactt agaacagcct atgggaagta gtcatgcatc
3661 tcaggtttgt tctgagacac ctgatgacct gttagatgat ggtgaaataa aggaagatac
3721 tagttttgct gaaaatgaca ttaaggaaag ttctgctgtt tttagcaaaa gcgtccagaa
3781 aggagagctt agcaggagtc ctagcccttt cacccataca catttggctc agggttaccg
3841 aagaggggcc aagaaattag agtcctcaga agagaactta tctagtgagg atgaagagct
3901 tccctgcttc caacacttgt tatttggtaa agtaaacaat ataccttctc agtctactag
3961 gcatagcacc gttgctaccg agtgtctgtc taagaacaca gaggagaatt tattatcatt
4021 gaagaatagc ttaaatgact gcagtaacca ggtaatattg gcaaaggcat ctcaggaaca
4081 tcaccttagt gaggaaacaa aatgttctgc tagcttgttt tcttcacagt gcagtgaatt
4141 ggaagacttg actgcaaata caaacaccca ggatcctttc ttgattggtt cttccaaaca
4201 aatgaggcat cagtctgaaa gccagggagt tggtctgagt gacaaggaat tggtttcaga
4261 tgatgaagaa agaggaacgg gcttggaaga aaataatcaa gaagagcaaa gcatggattc
4321 aaacttaggt gaagcagcat ctgggtgtga gagtgaaaca agcgtctctg aagactgctc
4381 agggctatcc tctcagagtg acattttaac cactcagcag agggatacca tgcaacataa
4441 cctgataaag ctccagcagg aaatggctga actagaagct gtgttagaac agcatgggag
4501 ccagccttct aacagctacc cttccatcat aagtgactct tctgcccttg aggacctgcg
4561 aaatccagaa caaagcacat cagaaaaaga ttcgcatata catggccaaa ggaacaactc
4621 catgttttct aaaaggccta gagaacatat atcagtatta acttcacaga aaagtagtga
4681 ataccctata agccagaatc cagaaggcct ttctgctgac aagtttgagg tgtctgcaga
4741 tagttctacc agtaaaaata aagaaccagg agtggaaagg tcatcccctt ctaaatgccc
4801 atcattagat gataggtggt acatgcacag ttgctctggg agtcttcaga atagaaacta
4861 cccatctcaa gaggagctca ttaaggttgt tgatgtggag gagcaacagc tggaagagtc
4921 tgggccacac gatttgacgg aaacatctta cttgccaagg caagatctag agggaacccc
4981 ttacctggaa tctggaatca gcctcttctc tgatgaccct gaatctgatc cttctgaaga
5041 cagagcccca gagtcagctc gtgttggcaa cataccatct tcaacctctg cattgaaagt
5101 tccccaattg aaagttgcag aatctgccca gagtccagct gctgctcata ctactgatac
5161 tgctgggtat aatgcaatgg aagaaagtgt gagcagggag aagccagaat tgacagcttc
5221 aacagaaagg gtcaacaaaa gaatgtccat ggtggtgtct ggcctgaccc cagaagaatt
5281 tatgctcgtg tacaagtttg ccagaaaaca ccacatcact ttaactaatc taattactga
5341 agagactact catgttgtta tgaaaacaga tgctgagttt gtgtgtgaac ggacactgaa
5401 atattttcta ggaattgcgg gaggaaaatg ggtagttagc tatttctggg tgacccagtc
5461 tattaaagaa agaaaaatgc tgaatgagca tgattttgaa gtcagaggag atgtggtcaa
5521 tggaagaaac caccaaggtc caaagcgagc aagagaatcc caggacagaa agatcttcag
5581 ggggctagaa atctgttgct atgggccctt caccaacatg cccacagatc aactggaatg
5641 gatggtacag ctgtgtggtg cttctgtggt gaaggagctt tcatcattca cccttggcac
5701 aggtgtccac ccaattgtgg ttgtgcagcc agatgcctgg acagaggaca atggcttcca
5761 tgcaattggg cagatgtgtg aggcacctgt ggtgacccga gagtgggtgt tggacagtgt
5821 agcactctac cagtgccagg agctggacac ctacctgata ccccagatcc cccacagcca
5881 ctactgactg cagccagcca caggtacaga gccacaggac cccaagaatg agcttacaaa
5941 gtggcctttc caggccctgg gagctcctct cactcttcag tccttctact gtcctggcta
6001 ctaaatattt tatgtacatc agcctgaaaa ggacttctgg ctatgcaagg gtcccttaaa
6061 gattttctgc ttgaagtctc ccttggaaat ctgccatgag cacaaaatta tggtaatttt
6121 tcacctgaga agattttaaa accatttaaa cgccaccaat tgagcaagat gctgattcat
6181 tatttatcag ccctattctt tctattcagg ctgttgttgg cttagggctg gaagcacaga
6241 gtggcttggc ctcaagagaa tagctggttt ccctaagttt acttctctaa aaccctgtgt
6301 tcacaaaggc agagagtcag acccttcaat ggaaggagag tgcttgggat cgattatgtg
6361 acttaaagtc agaatagtcc ttgggcagtt ctcaaatgtt ggagtggaac attggggagg
6421 aaattctgag gcaggtatta gaaatgaaaa ggaaacttga aacctgggca tggtggctca
6481 cgcctgtaat cccagcactt tgggaggcca aggtgggcag atcactggag gtcaggagtt
6541 cgaaaccagc ctggccaaca tggtgaaacc ccatctctac taaaaataca gaaattagcc
6601 ggtcatggtg gtggacacct gtaatcccag ctactcaggt ggctaaggca ggagaatcac
6661 ttcagcccgg gaggtggagg ttgcagtgag ccaagatcat accacggcac tccagcctgg
6721 gtgacagtga gactgtggct caaaaaaaaa aaaaaaaaaa ggaaaatgaa actagaagag
6781 atttctaaaa gtctgagata tatttgctag atttctaaag aatgtgttct aaaacagcag
6841 aagattttca agaaccggtt tccaaagaca gtcttctaat tcctcattag taataagtaa
6901 aatgtttatt gttgtagctc tggtatataa tccattcctc ttaaaatata agacctctgg
6961 catgaatatt tcatatctat aaaatgacag atcccaccag gaaggaagct gttgctttct
7021 ttgaggtgat ttttttcctt tgctccctgt tgctgaaacc atacagcttc ataaataatt
7081 ttgcttgctg aaggaagaaa aagtgttttt cataaaccca ttatccagga ctgtttatag
7141 ctgttggaag gactaggtct tccctagccc ccccagtgtg caagggcagt gaagacttga
7201 ttgtacaaaa tacgttttgt aaatgttgtg ctgttaacac tgcaaataaa cttggtagca
7261 aacacttcca aaaaaaaaaa aaaaaaa
Human BRCA1, transcript variant 3, is encoded by the nucleic acid sequence of
NCBI Accession No. NM_007297 and SEQ ID NO: 13).
      1 cttagcggta gccccttggt ttccgtggca acggaaaagc gcgggaatta cagataaatt
    61 aaaactgcga ctgcgcggcg tgagctcgct gagacttcct ggacggggga caggctgtgg
  121 ggtttctcag ataactgggc ccctgcgctc aggaggcctt caccctctgc tctggttcat
  181 tggaacagaa agaaatggat ttatctgctc ttcgcgttga agaagtacaa aatgtcatta
  241 atgctatgca gaaaatctta gagtgtccca tctgattttg catgctgaaa cttctcaacc
  301 agaagaaagg gccttcacag tgtcctttat gtaagaatga tataaccaaa aggagcctac
  361 aagaaagtac gagatttagt caacttgttg aagagctatt gaaaatcatt tgtgcttttc
  421 agcttgacac aggtttggag tatgcaaaca gctataattt tgcaaaaaag gaaaataact
  481 ctcctgaaca tctaaaagat gaagtttcta tcatccaaag tatgggctac agaaaccgtg
  541 ccaaaagact tctacagagt gaacccgaaa atccttcctt gcaggaaacc agtctcagtg
  601 tccaactctc taaccttgga actgtgagaa ctctgaggac aaagcagcgg atacaacctc
  661 aaaagacgtc tgtctacatt gaattgggat ctgattcttc tgaagatacc gttaataagg
  721 caacttattg cagtgtggga gatcaagaat tgttacaaat cacccctcaa ggaaccaggg
  781 atgaaatcag tttggattct gcaaaaaagg ctgcttgtga attttctgag acggatgtaa
  841 caaatactga acatcatcaa cccagtaata atgatttgaa caccactgag aagcgtgcag
  901 ctgagaggca tccagaaaag tatcagggta gttctgtttc aaacttgcat gtggagccat
  961 gtggcacaaa tactcatgcc agctcattac agcatgagaa cagcagttta ttactcacta
1021 aagacagaat gaatgtagaa aaggctgaat tctgtaataa aagcaaacag cctggcttag
1081 caaggagcca acataacaga tgggctggaa gtaaggaaac atgtaatgat aggcggactc
1141 ccagcacaga aaaaaaggta gatctgaatg ctgatcccct gtgtgagaga aaagaatgga
1201 ataagcagaa actgccatgc tcagagaatc ctagagatac tgaagatgtt ccttggataa
1261 cactaaatag cagcattcag aaagttaatg agtggttttc cagaagtgat gaactgttag
1321 gttctgatga ctcacatgat ggggagtctg aatcaaatgc caaagtagct gatgtattgg
1381 acgttctaaa tgaggtagat gaatattctg gttcttcaga gaaaatagac ttactggcca
1441 gtgatcctca tgaggcttta atatgtaaaa gtgaaagagt tcactccaaa tcagtagaga
1501 gtaatattga agacaaaata tttgggaaaa cctatcggaa gaaggcaagc ctccccaact
1561 taagccatgt aactgaaaat ctaattatag gagcatttgt tactgagcca cagataatac
1621 aagagcgtcc cctcacaaat aaattaaagc gtaaaaggag acctacatca ggccttcatc
1681 ctgaggattt tatcaagaaa gcagatttgg cagttcaaaa gactcctgaa atgataaatc
1741 agggaactaa ccaaacggag cagaatggtc aagtgatgaa tattactaat agtggtcatg
1801 agaataaaac aaaaggtgat tctattcaga atgagaaaaa tcctaaccca atagaatcac
1861 tcgaaaaaga atctgctttc aaaacgaaag ctgaacctat aagcagcagt ataagcaata
1921 tggaactcga attaaatatc cacaattcaa aagcacctaa aaagaatagg ctgaggagga
1981 agtcttctac caggcatatt catgcgcttg aactagtagt cagtagaaat ctaagcccac
2041 ctaattgtac tgaattgcaa attgatagtt gttctagcag tgaagagata aagaaaaaaa
2101 agtacaacca aatgccagtc aggcacagca gaaacctaca actcatggaa ggtaaagaac
2161 ctgcaactgg agccaagaag agtaacaagc caaatgaaca gacaagtaaa agacatgaca
2221 gcgatacttt cccagagctg aagttaacaa atgcacctgg ttcttttact aagtgttcaa
2281 ataccagtga acttaaagaa tttgtcaatc ctagccttcc aagagaagaa aaagaagaga
2341 aactagaaac agttaaagtg tctaataatg ctgaagaccc caaagatctc atgttaagtg
2401 gagaaagggt tttgcaaact gaaagatctg tagagagtag cagtatttca ttggtacctg
2461 gtactgatta tggcactcag gaaagtatct cgttactgga agttagcact ctagggaagg
2521 caaaaacaga accaaataaa tgtgtgagtc agtgtgcagc atttgaaaac cccaagggac
2581 taattcatgg ttgttccaaa gataatagaa atgacacaga aggctttaag tatccattgg
2641 gacatgaagt taaccacagt cgggaaacaa gcatagaaat ggaagaaagt gaacttgatg
2701 ctcagtattt gcagaataca ttcaaggttt caaagcgcca gtcatttgct ccgttttcaa
2761 atccaggaaa tgcagaagag gaatgtgcaa cattctctgc ccactctggg tccttaaaga
2821 aacaaagtcc aaaagtcact tttgaatgtg aacaaaagga agaaaatcaa ggaaagaatg
2881 agtctaatat caagcctgta cagacagtta atatcactgc aggctttcct gtggttggtc
2941 agaaagataa gccagttgat aatgccaaat gtagtatcaa aggaggctct aggttttgtc
3001 tatcatctca gttcagaggc aacgaaactg gactcattac tccaaataaa catggacttt
3061 tacaaaaccc atatcgtata ccaccacttt ttcccatcaa gtcatttgtt aaaactaaat
3121 gtaagaaaaa tctgctagag gaaaactttg aggaacattc aatgtcacct gaaagagaaa
3181 tgggaaatga gaacattcca agtacagtga gcacaattag ccgtaataac attagagaaa
3241 atgtttttaa agaagccagc tcaagcaata ttaatgaagt aggttccagt actaatgaag
3301 tgggctccag tattaatgaa ataggttcca gtgatgaaaa cattcaagca gaactaggta
3361 gaaacagagg gccaaaattg aatgctatgc ttagattagg ggttttgcaa cctgaggtct
3421 ataaacaaag tcttcctgga agtaattgta agcatcctga aataaaaaag caagaatatg
3481 aagaagtagt tcagactgtt aatacagatt tctctccata tctgatttca gataacttag
3541 aacagcctat gggaagtagt catgcatctc aggtttgttc tgagacacct gatgacctgt
3601 tagatgatgg tgaaataaag gaagatacta gttttgctga aaatgacatt aaggaaagtt
3661 ctgctgtttt tagcaaaagc gtccagaaag gagagcttag caggagtcct agccctttca
3721 cccatacaca tttggctcag ggttaccgaa gaggggccaa gaaattagag tcctcagaag
3781 agaacttatc tagtgaggat gaagagcttc cctgcttcca acacttgtta tttggtaaag
3841 taaacaatat accttctcag tctactaggc atagcaccgt tgctaccgag tgtctgtcta
3901 agaacacaga ggagaattta ttatcattga agaatagctt aaatgactgc agtaaccagg
3961 taatattggc aaaggcatct caggaacatc accttagtga ggaaacaaaa tgttctgcta
4021 gcttgttttc ttcacagtgc agtgaattgg aagacttgac tgcaaataca aacacccagg
4081 atcctttctt gattggttct tccaaacaaa tgaggcatca gtctgaaagc cagggagttg
4141 gtctgagtga caaggaattg gtttcagatg atgaagaaag aggaacgggc ttggaagaaa
4201 ataatcaaga agagcaaagc atggattcaa acttaggtga agcagcatct gggtgtgaga
4261 gtgaaacaag cgtctctgaa gactgctcag ggctatcctc tcagagtgac attttaacca
4321 ctcagcagag ggataccatg caacataacc tgataaagct ccagcaggaa atggctgaac
4381 tagaagctgt gttagaacag catgggagcc agccttctaa cagctaccct tccatcataa
4441 gtgactcttc tgcccttgag gacctgcgaa atccagaaca aagcacatca gaaaaagcag
4501 tattaacttc acagaaaagt agtgaatacc ctataagcca gaatccagaa ggcctttctg
4561 ctgacaagtt tgaggtgtct gcagatagtt ctaccagtaa aaataaagaa ccaggagtgg
4621 aaaggtcatc cccttctaaa tgcccatcat tagatgatag gtggtacatg cacagttgct
4681 ctgggagtct tcagaataga aactacccat ctcaagagga gctcattaag gttgttgatg
4741 tggaggagca acagctggaa gagtctgggc cacacgattt gacggaaaca tcttacttgc
4801 caaggcaaga tctagaggga accccttacc tggaatctgg aatcagcctc ttctctgatg
4861 accctgaatc tgatccttct gaagacagag ccccagagtc agctcgtgtt ggcaacatac
4921 catcttcaac ctctgcattg aaagttcccc aattgaaagt tgcagaatct gcccagagtc
4981 cagctgctgc tcatactact gatactgctg ggtataatgc aatggaagaa agtgtgagca
5041 gggagaagcc agaattgaca gcttcaacag aaagggtcaa caaaagaatg tccatggtgg
5101 tgtctggcct gaccccagaa gaatttatgc tcgtgtacaa gtttgccaga aaacaccaca
5161 tcactttaac taatctaatt actgaagaga ctactcatgt tgttatgaaa acagatgctg
5221 agtttgtgtg tgaacggaca ctgaaatatt ttctaggaat tgcgggagga aaatgggtag
5281 ttagctattt ctgggtgacc cagtctatta aagaaagaaa aatgctgaat gagcatgatt
5341 ttgaagtcag aggagatgtg gtcaatggaa gaaaccacca aggtccaaag cgagcaagag
5401 aatcccagga cagaaagatc ttcagggggc tagaaatctg ttgctatggg cccttcacca
5461 acatgcccac agatcaactg gaatggatgg tacagctgtg tggtgcttct gtggtgaagg
5521 agctttcatc attcaccctt ggcacaggtg tccacccaat tgtggttgtg cagccagatg
5581 cctggacaga ggacaatggc ttccatgcaa ttgggcagat gtgtgaggca cctgtggtga
5641 cccgagagtg ggtgttggac agtgtagcac tctaccagtg ccaggagctg gacacctacc
5701 tgatacccca gatcccccac agccactact gactgcagcc agccacaggt acagagccac
5761 aggaccccaa gaatgagctt acaaagtggc ctttccaggc cctgggagct cctctcactc
5821 ttcagtcctt ctactgtcct ggctactaaa tattttatgt acatcagcct gaaaaggact
5881 tctggctatg caagggtccc ttaaagattt tctgcttgaa gtctcccttg gaaatctgcc
5941 atgagcacaa aattatggta atttttcacc tgagaagatt ttaaaaccat ttaaacgcca
6001 ccaattgagc aagatgctga ttcattattt atcagcccta ttctttctat tcaggctgtt
6061 gttggcttag ggctggaagc acagagtggc ttggcctcaa gagaatagct ggtttcccta
6121 agtttacttc tctaaaaccc tgtgttcaca aaggcagaga gtcagaccct tcaatggaag
6181 gagagtgctt gggatcgatt atgtgactta aagtcagaat agtccttggg cagttctcaa
6241 atgttggagt ggaacattgg ggaggaaatt ctgaggcagg tattagaaat gaaaaggaaa
6301 cttgaaacct gggcatggtg gctcacgcct gtaatcccag cactttggga ggccaaggtg
6361 ggcagatcac tggaggtcag gagttcgaaa ccagcctggc caacatggtg aaaccccatc
6421 tctactaaaa atacagaaat tagccggtca tggtggtgga cacctgtaat cccagctact
6481 caggtggcta aggcaggaga atcacttcag cccgggaggt ggaggttgca gtgagccaag
6541 atcataccac ggcactccag cctgggtgac agtgagactg tggctcaaaa aaaaaaaaaa
6601 aaaaaggaaa atgaaactag aagagatttc taaaagtctg agatatattt gctagatttc
6661 taaagaatgt gttctaaaac agcagaagat tttcaagaac cggtttccaa agacagtctt
6721 ctaattcctc attagtaata agtaaaatgt ttattgttgt agctctggta tataatccat
6781 tcctcttaaa atataagacc tctggcatga atatttcata tctataaaat gacagatccc
6841 accaggaagg aagctgttgc tttctttgag gtgatttttt tcctttgctc cctgttgctg
6901 aaaccataca gcttcataaa taattttgct tgctgaagga agaaaaagtg tttttcataa
6961 acccattatc caggactgtt tatagctgtt ggaaggacta ggtcttccct agccccccca
7021 gtgtgcaagg gcagtgaaga cttgattgta caaaatacgt tttgtaaatg ttgtgctgtt
7081 aacactgcaa ataaacttgg tagcaaacac ttccaaaaaa aaaaaaaaaa aa
Human BRCA1, transcript variant 4, is encoded by the nucleic acid sequence of
NCBI Accession No. NM_007298 and SEQ ID NO: 14).
      1 ttcattggaa cagaaagaaa tggatttatc tgctcttcgc gttgaagaag tacaaaatgt
    61 cattaatgct atgcagaaaa tcttagagtg tcccatctgt ctggagttga tcaaggaacc
  121 tgtctccaca aagtgtgacc acatattttg caaattttgc atgctgaaac ttctcaacca
  181 gaagaaaggg ccttcacagt gtcctttatg taagaatgat ataaccaaaa ggagcctaca
  241 agaaagtacg agatttagtc aacttgttga agagctattg aaaatcattt gtgcttttca
  301 gcttgacaca ggtttggagt atgcaaacag ctataatttt gcaaaaaagg aaaataactc
  361 tcctgaacat ctaaaagatg aagtttctat catccaaagt atgggctaca gaaaccgtgc
  421 caaaagactt ctacagagtg aacccgaaaa tccttccttg caggaaacca gtctcagtgt
  481 ccaactctct aaccttggaa ctgtgagaac tctgaggaca aagcagcgga tacaacctca
  541 aaagacgtct gtctacattg aattgggatc tgattcttct gaagataccg ttaataaggc
  601 aacttattgc agtgtgggag atcaagaatt gttacaaatc acccctcaag gaaccaggga
  661 tgaaatcagt ttggattctg caaaaaaggc tgcttgtgaa ttttctgaga cggatgtaac
  721 aaatactgaa catcatcaac ccagtaataa tgatttgaac accactgaga agcgtgcagc
  781 tgagaggcat ccagaaaagt atcagggtga agcagcatct gggtgtgaga gtgaaacaag
  841 cgtctctgaa gactgctcag ggctatcctc tcagagtgac attttaacca ctcagcagag
  901 ggataccatg caacataacc tgataaagct ccagcaggaa atggctgaac tagaagctgt
  961 gttagaacag catgggagcc agccttctaa cagctaccct tccatcataa gtgactcttc
1021 tgcccttgag gacctgcgaa atccagaaca aagcacatca gaaaaagtat taacttcaca
1081 gaaaagtagt gaatacccta taagccagaa tccagaaggc ctttctgctg acaagtttga
1141 ggtgtctgca gatagttcta ccagtaaaaa taaagaacca ggagtggaaa ggtcatcccc
1201 ttctaaatgc ccatcattag atgataggtg gtacatgcac agttgctctg ggagtcttca
1261 gaatagaaac tacccatctc aagaggagct cattaaggtt gttgatgtgg aggagcaaca
1321 gctggaagag tctgggccac acgatttgac ggaaacatct tacttgccaa ggcaagatct
1381 agagggaacc ccttacctgg aatctggaat cagcctcttc tctgatgacc ctgaatctga
1441 tccttctgaa gacagagccc cagagtcagc tcgtgttggc aacataccat cttcaacctc
1501 tgcattgaaa gttccccaat tgaaagttgc agaatctgcc cagagtccag ctgctgctca
1561 tactactgat actgctgggt ataatgcaat ggaagaaagt gtgagcaggg agaagccaga
1621 attgacagct tcaacagaaa gggtcaacaa aagaatgtcc atggtggtgt ctggcctgac
1681 cccagaagaa tttatgctcg tgtacaagtt tgccagaaaa caccacatca ctttaactaa
1741 tctaattact gaagagacta ctcatgttgt tatgaaaaca gatgctgagt ttgtgtgtga
1801 acggacactg aaatattttc taggaattgc gggaggaaaa tgggtagtta gctatttctg
1861 ggtgacccag tctattaaag aaagaaaaat gctgaatgag catgattttg aagtcagagg
1921 agatgtggtc aatggaagaa accaccaagg tccaaagcga gcaagagaat cccaggacag
1981 aaagatcttc agggggctag aaatctgttg ctatgggccc ttcaccaaca tgcccacaga
2041 tcaactggaa tggatggtac agctgtgtgg tgcttctgtg gtgaaggagc tttcatcatt
2101 cacccttggc acaggtgtcc acccaattgt ggttgtgcag ccagatgcct ggacagagga
2161 caatggcttc catgcaattg ggcagatgtg tgaggcacct gtggtgaccc gagagtgggt
2221 gttggacagt gtagcactct accagtgcca ggagctggac acctacctga taccccagat
2281 cccccacagc cactactgac tgcagccagc cacaggtaca gagccacagg accccaagaa
2341 tgagcttaca aagtggcctt tccaggccct gggagctcct ctcactcttc agtccttcta
2401 ctgtcctggc tactaaatat tttatgtaca tcagcctgaa aaggacttct ggctatgcaa
2461 gggtccctta aagattttct gcttgaagtc tcccttggaa atctgccatg agcacaaaat
2521 tatggtaatt tttcacctga gaagatttta aaaccattta aacgccacca attgagcaag
2581 atgctgattc attatttatc agccctattc tttctattca ggctgttgtt ggcttagggc
2641 tggaagcaca gagtggcttg gcctcaagag aatagctggt ttccctaagt ttacttctct
2701 aaaaccctgt gttcacaaag gcagagagtc agacccttca atggaaggag agtgcttggg
2761 atcgattatg tgacttaaag tcagaatagt ccttgggcag ttctcaaatg ttggagtgga
2821 acattgggga ggaaattctg aggcaggtat tagaaatgaa aaggaaactt gaaacctggg
2881 catggtggct cacgcctgta atcccagcac tttgggaggc caaggtgggc agatcactgg
2941 aggtcaggag ttcgaaacca gcctggccaa catggtgaaa ccccatctct actaaaaata
3001 cagaaattag ccggtcatgg tggtggacac ctgtaatccc agctactcag gtggctaagg
3061 caggagaatc acttcagccc gggaggtgga ggttgcagtg agccaagatc ataccacggc
3121 actccagcct gggtgacagt gagactgtgg ctcaaaaaaa aaaaaaaaaa aaggaaaatg
3181 aaactagaag agatttctaa aagtctgaga tatatttgct agatttctaa agaatgtgtt
3241 ctaaaacagc agaagatttt caagaaccgg tttccaaaga cagtcttcta attcctcatt
3301 agtaataagt aaaatgttta ttgttgtagc tctggtatat aatccattcc tcttaaaata
3361 taagacctct ggcatgaata tttcatatct ataaaatgac agatcccacc aggaaggaag
3421 ctgttgcttt ctttgaggtg atttttttcc tttgctccct gttgctgaaa ccatacagct
3481 tcataaataa ttttgcttgc tgaaggaaga aaaagtgttt ttcataaacc cattatccag
3541 gactgtttat agctgttgga aggactaggt cttccctagc ccccccagtg tgcaagggca
3601 gtgaagactt gattgtacaa aatacgtttt gtaaatgttg tgctgttaac actgcaaata
3661 aacttggtag caaacacttc caaaaaaaaa aaaaaaaaa
Human BRCA1, transcript variant 5, is encoded by the nucleic acid sequence of
NCBI Accession No. NM_007299 and SEQ ID NO: 15).
      1 cttagcggta gccccttggt ttccgtggca acggaaaagc gcgggaatta cagataaatt
    61 aaaactgcga ctgcgcggcg tgagctcgct gagacttcct ggacggggga caggctgtgg
  121 ggtttctcag ataactgggc ccctgcgctc aggaggcctt caccctctgc tctggttcat
  181 tggaacagaa agaaatggat ttatctgctc ttcgcgttga agaagtacaa aatgtcatta
  241 atgctatgca gaaaatctta gagtgtccca tctgtctgga gttgatcaag gaacctgtct
  301 ccacaaagtg tgaccacata ttttgcaaat tttgcatgct gaaacttctc aaccagaaga
  361 aagggccttc acagtgtcct ttatgtaaga atgatataac caaaaggagc ctacaagaaa
  421 gtacgagatt tagtcaactt gttgaagagc tattgaaaat catttgtgct tttcagcttg
  481 acacaggttt ggagtatgca aacagctata attttgcaaa aaaggaaaat aactctcctg
  541 aacatctaaa agatgaagtt tctatcatcc aaagtatggg ctacagaaac cgtgccaaaa
  601 gacttctaca gagtgaaccc gaaaatcctt ccttgcagga aaccagtctc agtgtccaac
  661 tctctaacct tggaactgtg agaactctga ggacaaagca gcggatacaa cctcaaaaga
  721 cgtctgtcta cattgaattg ggatctgatt cttctgaaga taccgttaat aaggcaactt
  781 attgcagtgt gggagatcaa gaattgttac aaatcacccc tcaaggaacc agggatgaaa
  841 tcagtttgga ttctgcaaaa aaggctgctt gtgaattttc tgagacggat gtaacaaata
  901 ctgaacatca tcaacccagt aataatgatt tgaacaccac tgagaagcgt gcagctgaga
  961 ggcatccaga aaagtatcag ggtgaagcag catctgggtg tgagagtgaa acaagcgtct
1021 ctgaagactg ctcagggcta tcctctcaga gtgacatttt aaccactcag cagagggata
1081 ccatgcaaca taacctgata aagctccagc aggaaatggc tgaactagaa gctgtgttag
1141 aacagcatgg gagccagcct tctaacagct acccttccat cataagtgac tcttctgccc
1201 ttgaggacct gcgaaatcca gaacaaagca catcagaaaa agtattaact tcacagaaaa
1261 gtagtgaata ccctataagc cagaatccag aaggcctttc tgctgacaag tttgaggtgt
1321 ctgcagatag ttctaccagt aaaaataaag aaccaggagt ggaaaggtca tccccttcta
1381 aatgcccatc attagatgat aggtggtaca tgcacagttg ctctgggagt cttcagaata
1441 gaaactaccc atctcaagag gagctcatta aggttgttga tgtggaggag caacagctgg
1501 aagagtctgg gccacacgat ttgacggaaa catcttactt gccaaggcaa gatctagagg
1561 gaacccctta cctggaatct ggaatcagcc tcttctctga tgaccctgaa tctgatcctt
1621 ctgaagacag agccccagag tcagctcgtg ttggcaacat accatcttca acctctgcat
1681 tgaaagttcc ccaattgaaa gttgcagaat ctgcccagag tccagctgct gctcatacta
1741 ctgatactgc tgggtataat gcaatggaag aaagtgtgag cagggagaag ccagaattga
1801 cagcttcaac agaaagggtc aacaaaagaa tgtccatggt ggtgtctggc ctgaccccag
1861 aagaatttat gctcgtgtac aagtttgcca gaaaacacca catcacttta actaatctaa
1921 ttactgaaga gactactcat gttgttatga aaacagatgc tgagtttgtg tgtgaacgga
1981 cactgaaata ttttctagga attgcgggag gaaaatgggt agttagctat ttctgggtga
2041 cccagtctat taaagaaaga aaaatgctga atgagcatga ttttgaagtc agaggagatg
2101 tggtcaatgg aagaaaccac caaggtccaa agcgagcaag agaatcccag gacagaaaga
2161 tcttcagggg gctagaaatc tgttgctatg ggcccttcac caacatgccc acagggtgtc
2221 cacccaattg tggttgtgca gccagatgcc tggacagagg acaatggctt ccatgcaatt
2281 gggcagatgt gtgaggcacc tgtggtgacc cgagagtggg tgttggacag tgtagcactc
2341 taccagtgcc aggagctgga cacctacctg ataccccaga tcccccacag ccactactga
2401 ctgcagccag ccacaggtac agagccacag gaccccaaga atgagcttac aaagtggcct
2461 ttccaggccc tgggagctcc tctcactctt cagtccttct actgtcctgg ctactaaata
2521 ttttatgtac atcagcctga aaaggacttc tggctatgca agggtccctt aaagattttc
2581 tgcttgaagt ctcccttgga aatctgccat gagcacaaaa ttatggtaat ttttcacctg
2641 agaagatttt aaaaccattt aaacgccacc aattgagcaa gatgctgatt cattatttat
2701 cagccctatt ctttctattc aggctgttgt tggcttaggg ctggaagcac agagtggctt
2761 ggcctcaaga gaatagctgg tttccctaag tttacttctc taaaaccctg tgttcacaaa
2821 ggcagagagt cagacccttc aatggaagga gagtgcttgg gatcgattat gtgacttaaa
2881 gtcagaatag tccttgggca gttctcaaat gttggagtgg aacattgggg aggaaattct
2941 gaggcaggta ttagaaatga aaaggaaact tgaaacctgg gcatggtggc tcacgcctgt
3001 aatcccagca ctttgggagg ccaaggtggg cagatcactg gaggtcagga gttcgaaacc
3061 agcctggcca acatggtgaa accccatctc tactaaaaat acagaaatta gccggtcatg
3121 gtggtggaca cctgtaatcc cagctactca ggtggctaag gcaggagaat cacttcagcc
3181 cgggaggtgg aggttgcagt gagccaagat cataccacgg cactccagcc tgggtgacag
3241 tgagactgtg gctcaaaaaa aaaaaaaaaa aaaggaaaat gaaactagaa gagatttcta
3301 aaagtctgag atatatttgc tagatttcta aagaatgtgt tctaaaacag cagaagattt
3361 tcaagaaccg gtttccaaag acagtcttct aattcctcat tagtaataag taaaatgttt
3421 attgttgtag ctctggtata taatccattc ctcttaaaat ataagacctc tggcatgaat
3481 atttcatatc tataaaatga cagatcccac caggaaggaa gctgttgctt tctttgaggt
3541 gatttttttc ctttgctccc tgttgctgaa accatacagc ttcataaata attttgcttg
3601 ctgaaggaag aaaaagtgtt tttcataaac ccattatcca ggactgttta tagctgttgg
3661 aaggactagg tcttccctag cccccccagt gtgcaagggc agtgaagact tgattgtaca
3721 aaatacgttt tgtaaatgtt gtgctgttaa cactgcaaat aaacttggta gcaaacactt
3781 ccaaaaaaaa aaaaaaaaaa
Human BRCA1, transcript variant 6, is encoded by the nucleic acid sequence of
NCBI Accession No. NR_027676 and SEQ ID NO: 16).
      1 agataactgg gcccctgcgc tcaggaggcc ttcaccctct gctctgggta aaggtagtag
    61 agtcccggga aagggacagg gggcccaagt gatgctctgg ggtactggcg tgggagagtg
  121 gatttccgaa gctgacagat ggttcattgg aacagaaaga aatggattta tctgctcttc
  181 gcgttgaaga agtacaaaat gtcattaatg ctatgcagaa aatcttagag tgtcccatct
  241 gtctggagtt gatcaaggaa cctgtctcca caaagtgtga ccacatattt tgcaaatttt
  301 gcatgctgaa acttctcaac cagaagaaag ggccttcaca gtgtccttta tgagcctaca
  361 agaaagtacg agatttagtc aacttgttga agagctattg aaaatcattt gtgcttttca
  421 gcttgacaca ggtttggagt atgcaaacag ctataatttt gcaaaaaagg aaaataactc
  481 tcctgaacat ctaaaagatg aagtttctat catccaaagt atgggctaca gaaaccgtgc
  541 caaaagactt ctacagagtg aacccgaaaa tccttccttg gaaaccagtc tcagtgtcca
  601 actctctaac cttggaactg tgagaactct gaggacaaag cagcggatac aacctcaaaa
  661 gacgtctgtc tacattgaat tgggatctga ttcttctgaa gataccgtta ataaggcaac
  721 ttattgcagt gtgggagatc aagaattgtt acaaatcacc cctcaaggaa ccagggatga
  781 aatcagtttg gattctgcaa aaaaggctgc ttgtgaattt tctgagacgg atgtaacaaa
  841 tactgaacat catcaaccca gtaataatga tttgaacacc actgagaagc gtgcagctga
  901 gaggcatcca gaaaagtatc agggtagttc tgtttcaaac ttgcatgtgg agccatgtgg
  961 cacaaatact catgccagct cattacagca tgagaacagc agtttattac tcactaaaga
1021 cagaatgaat gtagaaaagg ctgaattctg taataaaagc aaacagcctg gcttagcaag
1081 gagccaacat aacagatggg ctggaagtaa ggaaacatgt aatgataggc ggactcccag
1141 cacagaaaaa aaggtagatc tgaatgctga tcccctgtgt gagagaaaag aatggaataa
1201 gcagaaactg ccatgctcag agaatcctag agatactgaa gatgttcctt ggataacact
1261 aaatagcagc attcagaaag ttaatgagtg gttttccaga agtgatgaac tgttaggttc
1321 tgatgactca catgatgggg agtctgaatc aaatgccaaa gtagctgatg tattggacgt
1381 tctaaatgag gtagatgaat attctggttc ttcagagaaa atagacttac tggccagtga
1441 tcctcatgag gctttaatat gtaaaagtga aagagttcac tccaaatcag tagagagtaa
1501 tattgaagac aaaatatttg ggaaaaccta tcggaagaag gcaagcctcc ccaacttaag
1561 ccatgtaact gaaaatctaa ttataggagc atttgttact gagccacaga taatacaaga
1621 gcgtcccctc acaaataaat taaagcgtaa aaggagacct acatcaggcc ttcatcctga
1681 ggattttatc aagaaagcag atttggcagt tcaaaagact cctgaaatga taaatcaggg
1741 aactaaccaa acggagcaga atggtcaagt gatgaatatt actaatagtg gtcatgagaa
1801 taaaacaaaa ggtgattcta ttcagaatga gaaaaatcct aacccaatag aatcactcga
1861 aaaagaatct gctttcaaaa cgaaagctga acctataagc agcagtataa gcaatatgga
1921 actcgaatta aatatccaca attcaaaagc acctaaaaag aataggctga ggaggaagtc
1981 ttctaccagg catattcatg cgcttgaact agtagtcagt agaaatctaa gcccacctaa
2041 ttgtactgaa ttgcaaattg atagttgttc tagcagtgaa gagataaaga aaaaaaagta
2101 caaccaaatg ccagtcaggc acagcagaaa cctacaactc atggaaggta aagaacctgc
2161 aactggagcc aagaagagta acaagccaaa tgaacagaca agtaaaagac atgacagcga
2221 tactttccca gagctgaagt taacaaatgc acctggttct tttactaagt gttcaaatac
2281 cagtgaactt aaagaatttg tcaatcctag ccttccaaga gaagaaaaag aagagaaact
2341 agaaacagtt aaagtgtcta ataatgctga agaccccaaa gatctcatgt taagtggaga
2401 aagggttttg caaactgaaa gatctgtaga gagtagcagt atttcattgg tacctggtac
2461 tgattatggc actcaggaaa gtatctcgtt actggaagtt agcactctag ggaaggcaaa
2521 aacagaacca aataaatgtg tgagtcagtg tgcagcattt gaaaacccca agggactaat
2581 tcatggttgt tccaaagata atagaaatga cacagaaggc tttaagtatc cattgggaca
2641 tgaagttaac cacagtcggg aaacaagcat agaaatggaa gaaagtgaac ttgatgctca
2701 gtatttgcag aatacattca aggtttcaaa gcgccagtca tttgctccgt tttcaaatcc
2761 aggaaatgca gaagaggaat gtgcaacatt ctctgcccac tctgggtcct taaagaaaca
2821 aagtccaaaa gtcacttttg aatgtgaaca aaaggaagaa aatcaaggaa agaatgagtc
2881 taatatcaag cctgtacaga cagttaatat cactgcaggc tttcctgtgg ttggtcagaa
2941 agataagcca gttgataatg ccaaatgtag tatcaaagga ggctctaggt tttgtctatc
3001 atctcagttc agaggcaacg aaactggact cattactcca aataaacatg gacttttaca
3061 aaacccatat cgtataccac cactttttcc catcaagtca tttgttaaaa ctaaatgtaa
3121 gaaaaatctg ctagaggaaa actttgagga acattcaatg tcacctgaaa gagaaatggg
3181 aaatgagaac attccaagta cagtgagcac aattagccgt aataacatta gagaaaatgt
3241 ttttaaagaa gccagctcaa gcaatattaa tgaagtaggt tccagtacta atgaagtggg
3301 ctccagtatt aatgaaatag gttccagtga tgaaaacatt caagcagaac taggtagaaa
3361 cagagggcca aaattgaatg ctatgcttag attaggggtt ttgcaacctg aggtctataa
3421 acaaagtctt cctggaagta attgtaagca tcctgaaata aaaaagcaag aatatgaaga
3481 agtagttcag actgttaata cagatttctc tccatatctg atttcagata acttagaaca
3541 gcctatggga agtagtcatg catctcaggt ttgttctgag acacctgatg acctgttaga
3601 tgatggtgaa ataaaggaag atactagttt tgctgaaaat gacattaagg aaagttctgc
3661 tgtttttagc aaaagcgtcc agaaaggaga gcttagcagg agtcctagcc ctttcaccca
3721 tacacatttg gctcagggtt accgaagagg ggccaagaaa ttagagtcct cagaagagaa
3781 cttatctagt gaggatgaag agcttccctg cttccaacac ttgttatttg gtaaagtaaa
3841 caatatacct tctcagtcta ctaggcatag caccgttgct accgagtgtc tgtctaagaa
3901 cacagaggag aatttattat cattgaagaa tagcttaaat gactgcagta accaggtaat
3961 attggcaaag gcatctcagg aacatcacct tagtgaggaa acaaaatgtt ctgctagctt
4021 gttttcttca cagtgcagtg aattggaaga cttgactgca aatacaaaca cccaggatcc
4081 tttcttgatt ggttcttcca aacaaatgag gcatcagtct gaaagccagg gagttggtct
4141 gagtgacaag gaattggttt cagatgatga agaaagagga acgggcttgg aagaaaataa
4201 tcaagaagag caaagcatgg attcaaactt aggtgaagca gcatctgggt gtgagagtga
4261 aacaagcgtc tctgaagact gctcagggct atcctctcag agtgacattt taaccactca
4321 gcagagggat accatgcaac ataacctgat aaagctccag caggaaatgg ctgaactaga
4381 agctgtgtta gaacagcatg ggagccagcc ttctaacagc tacccttcca tcataagtga
4441 ctcttctgcc cttgaggacc tgcgaaatcc agaacaaagc acatcagaaa aagcagtatt
4501 aacttcacag aaaagtagtg aataccctat aagccagaat ccagaaggcc tttctgctga
4561 caagtttgag gtgtctgcag atagttctac cagtaaaaat aaagaaccag gagtggaaag
4621 gtcatcccct tctaaatgcc catcattaga tgataggtgg tacatgcaca gttgctctgg
4681 gagtcttcag aatagaaact acccatctca agaggagctc attaaggttg ttgatgtgga
4741 ggagcaacag ctggaagagt ctgggccaca cgatttgacg gaaacatctt acttgccaag
4801 gcaagatcta gagggaaccc cttacctgga atctggaatc agcctcttct ctgatgaccc
4861 tgaatctgat ccttctgaag acagagcccc agagtcagct cgtgttggca acataccatc
4921 ttcaacctct gcattgaaag ttccccaatt gaaagttgca gaatctgccc agagtccagc
4981 tgctgctcat actactgata ctgctgggta taatgcaatg gaagaaagtg tgagcaggga
5041 gaagccagaa ttgacagctt caacagaaag ggtcaacaaa agaatgtcca tggtggtgtc
5101 tggcctgacc ccagaagaat ttatgctcgt gtacaagttt gccagaaaac accacatcac
5161 tttaactaat ctaattactg aagagactac tcatgttgtt atgaaaacag atgctgagtt
5221 tgtgtgtgaa cggacactga aatattttct aggaattgcg ggaggaaaat gggtagttag
5281 ctatttctgg gtgacccagt ctattaaaga aagaaaaatg ctgaatgagc atgattttga
5341 agtcagagga gatgtggtca atggaagaaa ccaccaaggt ccaaagcgag caagagaatc
5401 ccaggacaga aagatcttca gggggctaga aatctgttgc tatgggccct tcaccaacat
5461 gcccacagat caactggaat ggatggtaca gctgtgtggt gcttctgtgg tgaaggagct
5521 ttcatcattc acccttggca caggtgtcca cccaattgtg gttgtgcagc cagatgcctg
5581 gacagaggac aatggcttcc atgcaattgg gcagatgtgt gaggcacctg tggtgacccg
5641 agagtgggtg ttggacagtg tagcactcta ccagtgccag gagctggaca cctacctgat
5701 accccagatc ccccacagcc actactgact gcagccagcc acaggtacag agccacagga
5761 ccccaagaat gagcttacaa agtggccttt ccaggccctg ggagctcctc tcactcttca
5821 gtccttctac tgtcctggct actaaatatt ttatgtacat cagcctgaaa aggacttctg
5881 gctatgcaag ggtcccttaa agattttctg cttgaagtct cccttggaaa tctgccatga
5941 gcacaaaatt atggtaattt ttcacctgag aagattttaa aaccatttaa acgccaccaa
6001 ttgagcaaga tgctgattca ttatttatca gccctattct ttctattcag gctgttgttg
6061 gcttagggct ggaagcacag agtggcttgg cctcaagaga atagctggtt tccctaagtt
6121 tacttctcta aaaccctgtg ttcacaaagg cagagagtca gacccttcaa tggaaggaga
6181 gtgcttggga tcgattatgt gacttaaagt cagaatagtc cttgggcagt tctcaaatgt
6241 tggagtggaa cattggggag gaaattctga ggcaggtatt agaaatgaaa aggaaacttg
6301 aaacctgggc atggtggctc acgcctgtaa tcccagcact ttgggaggcc aaggtgggca
6361 gatcactgga ggtcaggagt tcgaaaccag cctggccaac atggtgaaac cccatctcta
6421 ctaaaaatac agaaattagc cggtcatggt ggtggacacc tgtaatccca gctactcagg
6481 tggctaaggc aggagaatca cttcagcccg ggaggtggag gttgcagtga gccaagatca
6541 taccacggca ctccagcctg ggtgacagtg agactgtggc tcaaaaaaaa aaaaaaaaaa
6601 aggaaaatga aactagaaga gatttctaaa agtctgagat atatttgcta gatttctaaa
6661 gaatgtgttc taaaacagca gaagattttc aagaaccggt ttccaaagac agtcttctaa
6721 ttcctcatta gtaataagta aaatgtttat tgttgtagct ctggtatata atccattcct
6781 cttaaaatat aagacctctg gcatgaatat ttcatatcta taaaatgaca gatcccacca
6841 ggaaggaagc tgttgctttc tttgaggtga tttttttcct ttgctccctg ttgctgaaac
6901 catacagctt cataaataat tttgcttgct gaaggaagaa aaagtgtttt tcataaaccc
6961 attatccagg actgtttata gctgttggaa ggactaggtc ttccctagcc cccccagtgt
7021 gcaagggcag tgaagacttg attgtacaaa atacgttttg taaatgttgt gctgttaaca
7081 ctgcaaataa acttggtagc aaacacttcc aaaaaaaaaa aaaaaaaa
```

BRCA1:miRNA Interactions

Significantly overexpressed miRNAs have been implicated as oncogenes that promote tumor development by negatively regulating tumor suppressor genes. As a tumor suppressor gene, one of the functions of BRCA1 may be repressing the expression of one or more miRNAs. For instance, MiR-7 is repressed by BRCA1 and is overexpressed in cells lacking BRCA1 (Table 1). FIG. 20 further demonstrates that miR-7 is highly expressed in breast cancer, and specifically, within the triple negative (TN) subtype. The studies provided herein demonstrate that patients who develop TN breast cancer often carry rare haplotypes that contain the rs1060915 SNP. Accordingly, the presence of this SNP prevents miR-7 from binding to BRCA1 (FIG. 21).

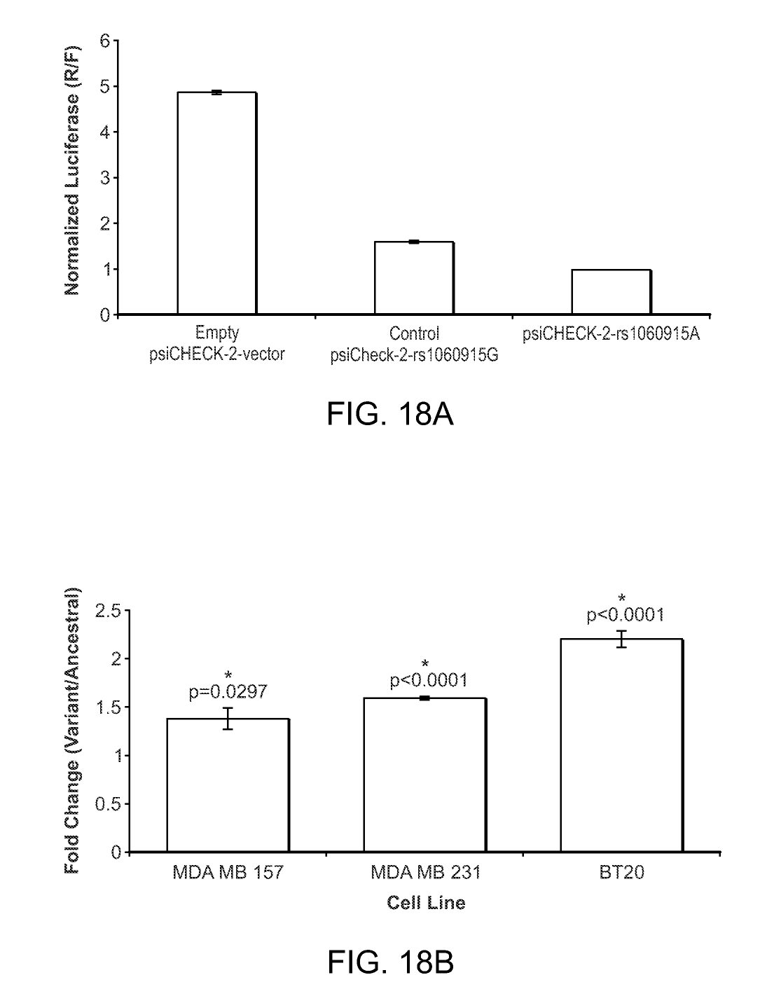

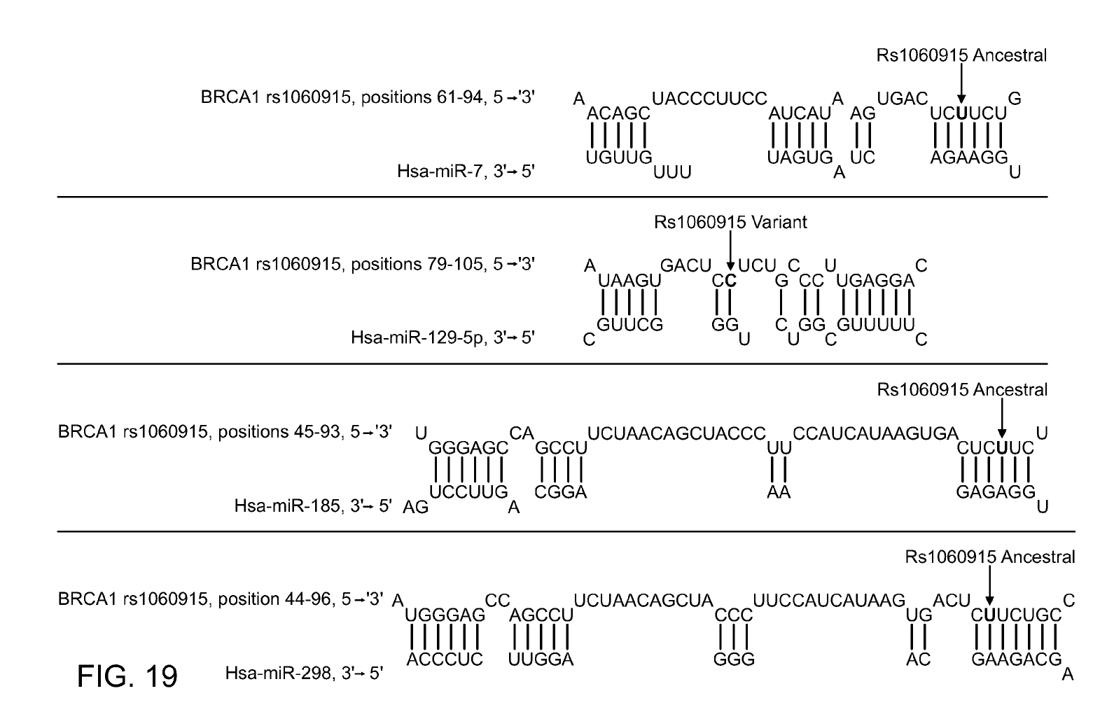

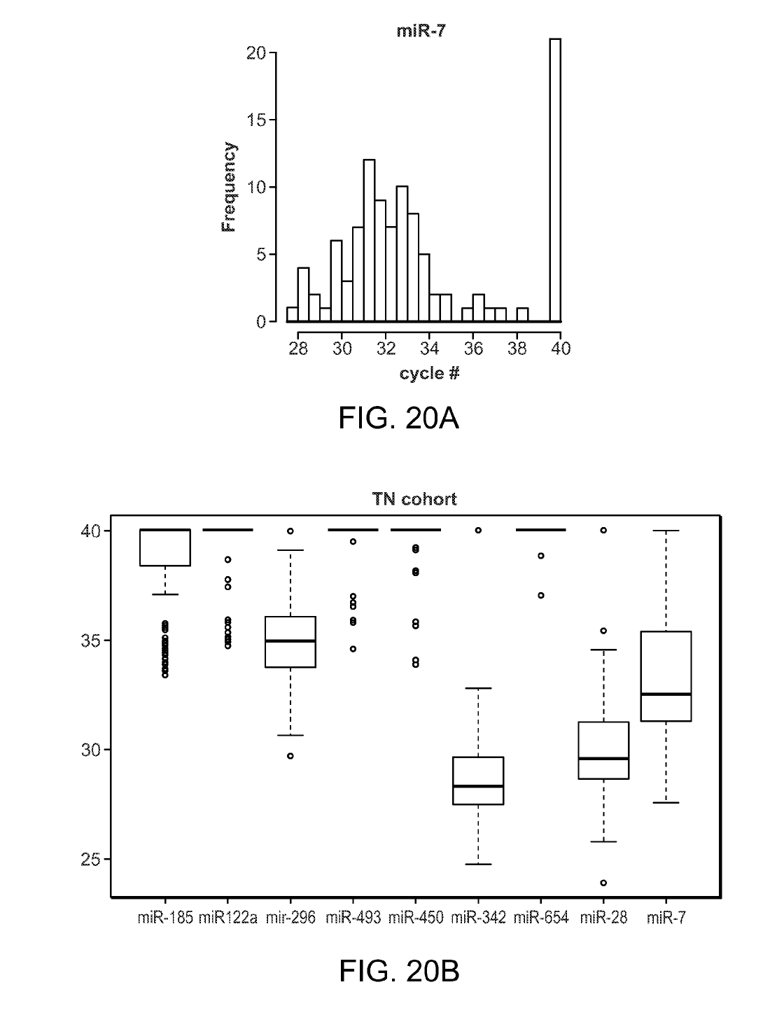

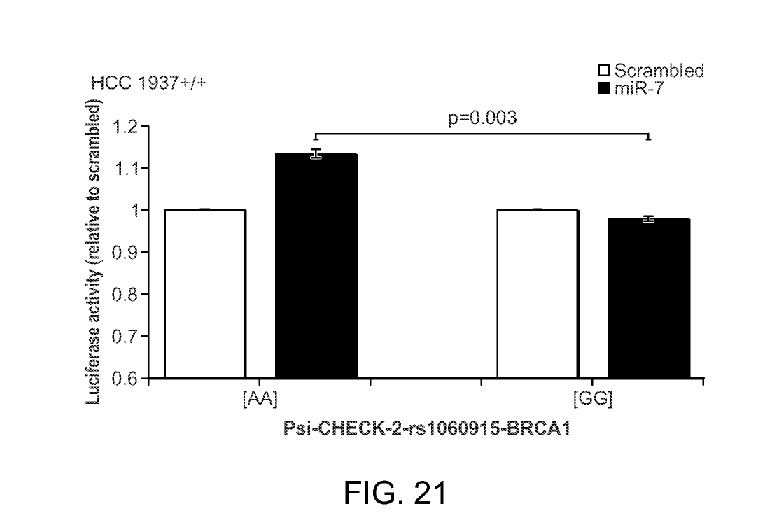

MiR-7 may be protective against breast cancer. Although the mechanism appears to be counterintuitive to the concept that miRNAs repress gene expression, when the miR-7 binding site is intact and miR-7 binds to BRCA1, expression of BRCA1 is higher, and therefore, the cell containing the BRCA1 contains more functional protein. MiRNAs binding within exons has been reported to have such effects. When the rs1060915 SNP is present in BRCA1, miR-7 is prevented from binding, expression levels of BRCA1 fall, and, consequently, the cell has less functional protein. Thus, rs1060915 regulatory element of expression that is contained within the BRCA1 gene (FIG. 18A-B).

With less available or functional BRCA1 protein, the DNA repair pathways that protect cells from DNA synthesis errors and unregulated proliferation are impaired. Thus, the risk of developing cancer is increased.

TABLE 1
Top 10 miRNAs repressed by BRCA1.

| | | BRCA+/BRCA− | |
|---|---|---|---|
| | | (Fold Change | |
| Number | miRNA | in Expression) | p-value |
| 1 | miR-19a | 1/11.2 | 5.17E−03 |
| 2 | miR-18b | 1/5.3 | 3.65E−03 |
| 3 | miR-19b | 1/4.2 | 2.27E−04 |
| 4 | miR-146-5p | 1/3.9 | 3.15E−05 |
| 5 | miR-18a | 1/3.8 | 4.28E−04 |
| 6 | miR-365 | 1/3.4 | 2.02E−03 |
| 7 | miR-210 | 1/3.1 | 1.46E−03 |
| 8 | miR-7 | 1/2.2 | 5.13E−03 |
| 9 | miR-151-3p | 1/2.2 | 1.18E−03 |
| 10 | miR-1180 | 1/2.2 | 3.25E−03 |

MiR-7 is repressed by BRCA1.
Expression of cellular mRNA levels analyzed in HCC1937 cells post-transfection with either wild type BRCA1 or vector control.
All of the listed miRNAs were expressed at higher levels in the cells lacking BRCA1.

### Isolated Nucleic Acid Molecules

The present invention provides isolated nucleic acid molecules that contain one or more SNPs. Isolated nucleic acid molecules containing one or more SNPs disclosed herein may be interchangeably referred to throughout the present text as “SNP-containing nucleic acid molecules”. Isolated nucleic acid molecules may optionally encode a full-length variant protein or fragment thereof. The isolated nucleic acid molecules of the present invention also include probes and primers (which are described in greater detail below in the section entitled “SNP Detection Reagents”), which may be used for assaying the disclosed SNPs, and isolated full-length genes, transcripts, cDNA molecules, and fragments thereof, which may be used for such purposes as expressing an encoded protein.

As used herein, an “isolated nucleic acid molecule” generally is one that contains a SNP of the present invention or one that hybridizes to such molecule such as a nucleic acid with a complementary sequence, and is separated from most other nucleic acids present in the natural source of the nucleic acid molecule. Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. A nucleic acid molecule can be fused to other coding or regulatory sequences and still be considered “isolated”. Nucleic acid molecules present in non-human transgenic animals, which do not naturally occur in the animal, are also considered “isolated”. For example, recombinant DNA molecules contained in a vector are considered “isolated”. Further examples of “isolated” DNA molecules include recombinant DNA molecules maintained in heterologous host cells, and purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. Preferably the flanking sequence is up to about 500, 300, 100, 60, 50, 30, 25, 20, 15, 10, 8, or 4 nucleotides (or any other length in-between) on either side of a SNP position, or as long as the full-length gene, entire coding, or non-coding sequence (or any portion thereof such as an exon, intron, or a 5′ or 3′ untranslated region), especially if the SNP-containing nucleic acid molecule is to be used to produce a protein or protein fragment.

For full-length genes and entire protein-coding sequences, a SNP flanking sequence can be, for example, up to about 5 KB, 4 KB, 3 KB, 2 KB, or 1 KB on either side of the SNP. Furthermore, in such instances, the isolated nucleic acid molecule comprises exonic sequences (including protein-coding and/or non-coding exonic sequences), but may also include intronic sequences and untranslated regulatory sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can comprise, for example, a full-length gene or transcript, such as a gene isolated from genomic DNA (e.g., by cloning or PCR amplification), a cDNA molecule, or an mRNA transcript molecule. Furthermore, fragments of such full-length genes and transcripts that contain one or more SNPs disclosed herein are also encompassed by the present invention.

Thus, the present invention also encompasses fragments of the nucleic acid sequences and their complements. A fragment typically comprises a contiguous nucleotide sequence at least about 8 or more nucleotides, more preferably at least about 10 or more nucleotides, and even more preferably at least about 16 or more nucleotides. Further, a fragment could comprise at least about 18, 20, 21, 22, 25, 30, 40, 50, 60, 100, 250 or 500 (or any other number in-between) nucleotides in length. The length of the fragment will be based on its intended use. Such fragments can be isolated using nucleotide sequences such as, but not limited to, SEQ ID NOs: 11-16 for the synthesis of a polynucleotide probe. A labeled probe can then be used, for example, to screen a cDNA library, genomic DNA library, or mRNA to isolate nucleic acid corresponding to the region of interest. Further, primers can be used in amplification reactions, such as for purposes of assaying one or more SNPs sites or for cloning specific regions of a gene.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein. Such primers may be used to amplify DNA of any length so long that it contains the SNP of interest in its sequence.

As used herein, an “amplified polynucleotide” of the invention is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 10 nucleotides in length. More typically, an amplified polynucleotide is at least about 16 nucleotides in length. In a preferred embodiment of the invention, an amplified polynucleotide is at least about 20 nucleotides in length. In a more preferred embodiment of the invention, an amplified polynucleotide is at least about 21, 22, 23, 24, 25, 30, 35, 40, 45, 50, or 60 nucleotides in length. In yet another preferred embodiment of the invention, an amplified polynucleotide is at least about 100, 200, or 300 nucleotides in length. While the total length of an amplified polynucleotide of the invention can be as long as an exon, an intron, a 5′ UTR, a 3′ UTR, or the entire gene where the SNP of interest resides, an amplified product is typically no greater than about 1,000 nucleotides in length (although certain amplification methods may generate amplified products greater than 1000 nucleotides in length). More preferably, an amplified polynucleotide is not greater than about 600 nucleotides in length. It is understood that irrespective of the length of an amplified polynucleotide, a SNP of interest may be located anywhere along its sequence.

Such a product may have additional sequences on its 5′ end or 3′ end or both. In another embodiment, the amplified product is about 101 nucleotides in length, and it contains a SNP disclosed herein. Preferably, the SNP is located at the middle of the amplified product (e.g., at position 101 in an amplified product that is 201 nucleotides in length, or at position 51 in an amplified product that is 101 nucleotides in length), or within 1, 2, 3, 4, 5, 6, 7, 8, 9, 10, 12, 15, or 20 nucleotides from the middle of the amplified product (however, as indicated above, the SNP of interest may be located anywhere along the length of the amplified product).

The present invention provides isolated nucleic acid molecules that comprise, consist of, or consist essentially of one or more polynucleotide sequences that contain one or more SNPs disclosed herein, complements thereof, and SNP-containing fragments thereof.

A nucleic acid molecule consists of a nucleotide sequence when the nucleotide sequence is the complete nucleotide sequence of the nucleic acid molecule.

A nucleic acid molecule consists essentially of a nucleotide sequence when such a nucleotide sequence is present with only a few additional nucleotide residues in the final nucleic acid molecule.

A nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated is provided below, and such techniques are well known to those of ordinary skill in the art (Sambrook and Russell, 2000, Molecular Cloning: A Laboratory Manual, Cold Spring Harbor Press, NY).

The isolated nucleic acid molecules include, but are not limited to, nucleic acid molecules having a sequence encoding a peptide alone, a sequence encoding a mature peptide and additional coding sequences such as a leader or secretory sequence (e.g., a pre-pro or pro-protein sequence), a sequence encoding a mature peptide with or without additional coding sequences, plus additional non-coding sequences, for example introns and non-coding 5′ and 3′ sequences such as transcribed but untranslated sequences that play a role in, for example, transcription, mRNA processing (including splicing and polyadenylation signals), ribosome binding, and/or stability of mRNA. In addition, the nucleic acid molecules may be fused to heterologous marker sequences encoding, for example, a peptide that facilitates purification.

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof (Sambrook and Russell, 2000, Molecular Cloning: A Laboratory Manual, Cold Spring Harbor Press, NY). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, e.g., Corey, “Peptide nucleic acids: expanding the scope of nucleic acid recognition”, Trends Biotechnol. 1997 June; 15(6):224-9, and Hyrup et al., “Peptide nucleic acids (PNA): synthesis, properties and potential applications”, Bioorg Med. Chem. 1996 January; 4(1):5-23). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System, and the sequence information provided herein.

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs identified in SEQ ID NOs: 21, 26 and 27. Furthermore, kits/systems (such as beads, arrays, etc.) that include these analogs are also encompassed by the present invention. For example, PNA oligomers that are based on the polymorphic sequences of the present invention are specifically contemplated. PNA oligomers are analogs of DNA in which the phosphate backbone is replaced with a peptide-like backbone (Lagriffoul et al., Bioorganic & Medicinal Chemistry Letters, 4: 1081-1082 (1994), Petersen et al., Bioorganic & Medicinal Chemistry Letters, 6: 793-796 (1996), Kumar et al., Organic Letters 3(9): 1269-1272 (2001), WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules, SNP-containing nucleic acid molecules, SNP detection reagents (e.g., probes and primers), oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002).

Further variants of the nucleic acid molecules including, but not limited to those identified as SEQ ID NOs: 11-16, such as naturally occurring allelic variants (as well as orthologs and paralogs) and synthetic variants produced by mutagenesis techniques, can be identified and/or produced using methods well known in the art. Such further variants can comprise a nucleotide sequence that shares at least 70-80%, 80-85%, 85-90%, 91%, 92%, 93%, 94%, 95%, 96%, 97%, 98%, or 99% sequence identity with a nucleic acid sequence disclosed as SEQ ID NOs: 11-16 (or a fragment thereof) and that includes a novel SNP allele. Thus, the present invention specifically contemplates isolated nucleic acid molecule that have a certain degree of sequence variation compared with the sequences of SEQ ID NOs: 11-16, but that contain a novel SNP allele.

The comparison of sequences and determination of percent identity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In a preferred embodiment, the percent identity between two amino acid sequences is determined using the Needleman and Wunsch algorithm (J. Mol. Biol. (48):444-453 (1970)) which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6.

In yet another preferred embodiment, the percent identity between two nucleotide sequences is determined using the GAP program in the GCG software package (Devereux, J., et al., Nucleic Acids Res. 12(1):387 (1984)), using a NWSgapdna.CMP matrix and a gap weight of 40, 50, 60, 70, or 80 and a length weight of 1, 2, 3, 4, 5, or 6.

In another embodiment, the percent identity between two amino acid or nucleotide sequences is determined using the algorithm of E. Myers and W. Miller (CABIOS, 4:11-17 (1989)) which has been incorporated into the ALIGN program (version 2.0), using a PAM120 weight residue table, a gap length penalty of 12, and a gap penalty of 4.

The nucleotide and amino acid sequences of the present invention can further be used as a “query sequence” to perform a search against sequence databases to, for example, identify other family members or related sequences. Such searches can be performed using the NBLAST and XBLAST programs (version 2.0) of Altschul, et al. (J. Mol. Biol. 215:403-10 (1990)). BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (e.g., XBLAST and NBLAST) can be used. In addition to BLAST, examples of other search and sequence comparison programs used in the art include, but are not limited to, FASTA (Pearson, Methods Mol. Biol. 25, 365-389 (1994)) and KERR (Dufresne et al., Nat Biotechnol 2002 December; 20(12): 1269-71). For further information regarding bioinformatics techniques, see Current Protocols in Bioinformatics, John Wiley & Sons, Inc., N.Y.

### SNP Detection Reagents

In a specific aspect of the present invention, the sequences disclosed herein can be used for the design of SNP detection reagents. In a preferred embodiment, sequences of SEQ ID NOs: 11-16 are used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagents hybridize to a target SNP-containing nucleic acid molecule by complementary base-pairing in a sequence specific manner, and discriminates the target variant sequence from other nucleic acid sequences such as an art-known form in a test sample. In a preferred embodiment, such a probe can differentiate between nucleic acids having a particular nucleotide (allele) at a target SNP position from other nucleic acids that have a different nucleotide at the same target SNP position. In addition, a detection reagent may hybridize to a specific region 5′ and/or 3′ to a SNP position, particularly a region corresponding the 3′UTR. Another example of a detection reagent is a primer which acts as an initiation point of nucleotide extension along a complementary strand of a target polynucleotide. The SNP sequence information provided herein is also useful for designing primers, e.g. allele-specific primers, to amplify (e.g., using PCR) any SNP of the present invention.

In one preferred embodiment of the invention, a SNP detection reagent is an isolated or synthetic DNA or RNA polynucleotide probe or primer or PNA oligomer, or a combination of DNA, RNA and/or PNA, which hybridizes to a segment of a target nucleic acid molecule containing a SNP located within a LCS. A detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

A probe or primer typically is a substantially purified oligonucleotide or PNA oligomer. Such oligonucleotide typically comprises a region of complementary nucleotide sequence that hybridizes under stringent conditions to at least about 8, 10, 12, 16, 18, 20, 21, 22, 25, 30, 40, 50, 60, 100 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

It will be apparent to one of skill in the art that such primers and probes are directly useful as reagents for genotyping the SNPs of the present invention, and can be incorporated into any kit/system format.

In order to produce a probe or primer specific for a target SNP-containing sequence, the gene/transcript and/or context sequence surrounding the SNP of interest is typically examined using a computer algorithm which starts at the 5′ or at the 3′ end of the nucleotide sequence. Typical algorithms will then identify oligomers of defined length that are unique to the gene/SNP context sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length (see the section below entitled “SNP Detection Kits and Systems”).

For analyzing SNPs, it may be appropriate to use oligonucleotides specific for alternative SNP alleles. Such oligonucleotides that detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235,726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the conditions under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Prehybridization with a solution containing 5.times. standard saline phosphate EDTA (SSPE), 0.5% NaDodSO.sub.4 (SDS) at 55.degree. C., and incubating probe with target nucleic acid molecules in the same solution at the same temperature, followed by washing with a solution containing 2.times.SSPE, and 0.1% SDS at 55.degree. C. or room temperature.

Moderate stringency hybridization conditions may be used for allele-specific primer extension reactions with a solution containing, e.g., about 50 mM KCl at about 46.degree. C. Alternatively, the reaction may be carried out at an elevated temperature such as 60.degree. C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46.degree. C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer. In a specific preferred embodiment which is particularly suitable for use in an oligonucleotide ligation assay (U.S. Pat. No. 4,988,617), the 3′ most nucleotide of the probe aligns with the SNP position in the target sequence.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22:1859; and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position.

Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity (Gibbs, 1989, Nucleic Acid Res. 17 2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). This PCR-based assay can be utilized as part of the TaqMan assay, described below.

In a specific embodiment of the invention, a primer of the invention contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

In another embodiment of the invention, a SNP detection reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment of the invention, the detection reagent may be further labeled with a quencher dye such as Tamra, especially when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl. 4:357-362; Tyagi et al., 1996, Nature Biotechnology 14: 303-308; Nazarenko et al., 1997, Nucl. Acids Res. 25:2516-2521; U.S. Pat. Nos. 5,866,336 and 6,117,635).

The detection reagents of the invention may also contain other labels, including but not limited to, biotin for streptavidin binding, hapten for antibody binding, and oligonucleotide for binding to another complementary oligonucleotide such as pairs of zipcodes.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the 3′-most end of the primer extension product, and in which the ddNTP corresponds to a SNP disclosed herein, is a composition that is encompassed by the present invention). Thus, reagents that bind to a nucleic acid molecule in a region adjacent to a SNP site, even though the bound sequences do not necessarily include the SNP site itself, are also encompassed by the present invention.

### SNP Detection Kits and Systems

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics”, Biotechnol Annu Rev. 2002; 8:85-101; Sosnowski et al., “Active microelectronic array system for DNA hybridization, genotyping and pharmacogenomic applications”, Psychiatr Genet. 2002 December; 12(4):181-92; Heller, “DNA microarray technology: devices, systems, and applications”, Annu Rev Biomed Eng. 2002; 4:129-53. Epub 2002 Mar. 22; Kolchinsky et al., “Analysis of SNPs and other genomic variations using gel-based chips”, Hum Mutat. 2002 April; 19(4):343-60; and McGall et al., “High-density genechip oligonucleotide probe arrays”, Adv Biochem Eng Biotechnol. 2002; 77:21-42.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular areas along the length of a target gene/transcript sequence, particularly areas corresponding to one or more SNPs. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, N.Y. (1989), 6.3.1-6.3.6.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. published application US2002/0110828 discloses methods and compositions for microarray controls.

In one embodiment of the invention, a nucleic acid array can comprise an array of probes of about 15-25 nucleotides in length. In further embodiments, a nucleic acid array can comprise any number of probes, in which at least one probe is capable of detecting the a SNP, and/or at least one probe comprises a fragment of one of the sequences selected from the group consisting of those disclosed in the Sequence Listing, sequences complementary thereto, and fragment thereof comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a novel SNP allele. In some embodiments, the nucleotide complementary to the SNP site is within 5, 4, 3, 2, or 1 nucleotide from the center of the probe, more preferably at the center of said probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM 6700, and Roche Molecular Systems' COBAS AmpliPrep System.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more SNPs of the present invention, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development”, Adv Drug Deliv Rev. 2003 Feb. 24; 55(3):349-77). In such microfluidic devices, the containers may be referred to as, for example, microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, are exemplary kits/systems of the present invention for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073, Dubrow et al., and U.S. Pat. No. 6,156,181, Parce et al.

For genotyping SNPs, an exemplary microfluidic system may integrate, for example, nucleic acid amplification, primer extension, capillary electrophoresis, and a detection method such as laser induced fluorescence detection. In a first step of an exemplary process for using such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted SNP. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. Such an exemplary microchip can be used to process, for example, at least 96 to 384 samples, or more, in parallel.

### Uses of Nucleic Acid Molecules

The nucleic acid molecules of the present invention have a variety of uses, especially in the assessing the risk of developing a disorder. Exemplary disorders include but are not limited to, inflammatory, degenerative, metabolic, proliferative, circulatory, cognitive, reproductive, and behavioral disorders. In a preferred embodiment of the invention the disorder is cancer. For example, the nucleic acid molecules are useful as hybridization probes, such as for genotyping SNPs in messenger RNA, transcript, cDNA, genomic DNA, amplified DNA or other nucleic acid molecules, and for isolating full-length cDNA and genomic clones.

A probe can hybridize to any nucleotide sequence along the entire length of a LCS-containing nucleic acid molecule. Preferably, a probe hybridizes to a SNP-containing target sequence in a sequence-specific manner such that it distinguishes the target sequence from other nucleotide sequences which vary from the target sequence only by which nucleotide is present at the SNP site. Such a probe is particularly useful for detecting the presence of a SNP-containing nucleic acid in a test sample, or for determining which nucleotide (allele) is present at a particular SNP site (i.e., genotyping the SNP site).

A nucleic acid hybridization probe may be used for determining the presence, level, form, and/or distribution of nucleic acid expression. The nucleic acid whose level is determined can be DNA or RNA. Accordingly, probes specific for the SNPs described herein can be used to assess the presence, expression and/or gene copy number in a given cell, tissue, or organism. These uses are relevant for diagnosis of disorders involving an increase or decrease in gene expression relative to normal levels. In vitro techniques for detection of mRNA include, for example, Northern blot hybridizations and in situ hybridizations. In vitro techniques for detecting DNA include Southern blot hybridizations and in situ hybridizations (Sambrook and Russell, 2000, Molecular Cloning: A Laboratory Manual, Cold Spring Harbor Press, Cold Spring Harbor, N.Y.).

Thus, the nucleic acid molecules of the invention can be used as hybridization probes to detect the SNPs disclosed herein, thereby determining whether an individual with the polymorphisms is at risk for developing a disorder. Detection of a SNP associated with a disease phenotype provides a prognostic tool for an active disease and/or genetic predisposition to the disease.

The nucleic acid molecules of the invention are also useful for designing ribozymes corresponding to all, or a part, of an mRNA molecule expressed from a SNP-containing nucleic acid molecule described herein.

The nucleic acid molecules of the invention are also useful for constructing transgenic animals expressing all, or a part, of the nucleic acid molecules and variant peptides. The production of recombinant cells and transgenic animals having nucleic acid molecules which contain a SNP disclosed herein allow, for example, effective clinical design of treatment compounds and dosage regimens.

### SNP Genotyping Methods

The process of determining which specific nucleotide (i.e., allele) is present at each of one or more SNP positions is referred to as SNP genotyping. The present invention provides methods of SNP genotyping, such as for use in screening for a variety of disorders, or determining predisposition thereto, or determining responsiveness to a form of treatment, or prognosis, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J Pharmacogenomics. 2002; 2(3):197-205; and Kwok, “Methods for genotyping single nucleotide polymorphisms”, Annu Rev Genomics Hum Genet. 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in prognostic assays for a variety of disorders including cancer, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

The identity of polymorphisms may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nuci. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruano et al., Nucl. Acids Res. 17:8392, 1989; Ruano et al., Nucl. Acids Res. 19, 6877-6882, 1991; WO 93/22456; Turki et al., J. Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

Another preferred method for genotyping the SNPs of the present invention is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5,494,810, 5,830,711, and 6054564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application US01/17329 (and Ser. No. 09/584,905) describes OLA (or LDR) followed by PCR, wherein zipcodes are incorporated into OLA probes, and amplified PCR products are determined by electrophoretic or universal zipcode array readout; U.S. application 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry”, Rapid Commun Mass Spectrom. 2003; 17(11):1195-202.

The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1:144-153; Storm et al., “MALDI-TOF mass spectrometry-based SNP genotyping”, Methods Mol. Biol. 2003; 212:241-62; Jurinke et al., “The use of MassARRAY technology for high throughput genotyping”, Adv Biochem Eng Biotechnol. 2002; 77:57-74; and Jurinke et al., “Automated genotyping using the DNA MassArray technology”, Methods Mol. Biol. 2002; 187:179-92.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), is commonly used in the art for automated sequencing.

Other methods that can be used to genotype the SNPs of the present invention include single-strand conformational polymorphism (SSCP), and denaturing gradient gel electrophoresis (DGGE) (Myers et al., Nature 313:495 (1985)). SSCP identifies base differences by alteration in electrophoretic migration of single stranded PCR products, as described in Orita et al., Proc. Nat. Acad. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W. H. Freeman and Co, New York, 1992, Chapter 7).

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described below. Examples of such applications include, but are not limited to, SNP-disease association analysis, disease predisposition screening, disease diagnosis, disease prognosis, disease progression monitoring, determining therapeutic strategies based on an individual's genotype (“pharmacogenomics”), developing therapeutic agents based on SNP genotypes associated with a disease or likelihood of responding to a drug, stratifying a patient population for clinical trial for a treatment regimen, and predicting the likelihood that an individual will experience toxic side effects from a therapeutic agent.

### Disease Screening Assays

Information on association/correlation between genotypes and disease-related phenotypes can be exploited in several ways. For example, in the case of a highly statistically significant association between one or more SNPs with predisposition to a disease for which treatment is available, detection of such a genotype pattern in an individual may justify immediate administration of treatment, or at least the institution of regular monitoring of the individual. In the case of a weaker but still statistically significant association between a SNP and a human disease, immediate therapeutic intervention or monitoring may not be justified after detecting the susceptibility allele or SNP. Nevertheless, the subject can be motivated to begin simple life-style changes (e.g., diet, exercise, quit smoking, increased monitoring/examination) that can be accomplished at little or no cost to the individual but would confer potential benefits in reducing the risk of developing conditions for which that individual may have an increased risk by virtue of having the susceptibility allele(s).

In one aspect, the invention provides methods of identifying SNPs which increase the risk, susceptibility, or probability of developing a disease such as a cell proliferative disorder (e.g. cancer). In a further aspect, the invention provides methods for identifying a subject at risk for developing a disease, determining the prognosis a disease or predicting the onset of a disease. For example, a subject's risk of developing a cell proliferative disease, the prognosis of an individual with a disease, or the predicted onset of a cell proliferative disease is are determined by detecting a mutation in the 3′ untranslated region (UTR) of BRCA1. Identification of the mutation indicates an increases risk of developing a cell proliferative disorder, poor prognosis or an earlier onset of developing a cell proliferative disorder.

The mutation is for example a deletion, insertion, inversion, substitution, frameshift or recombination. The mutation modulates, e.g. increases or decreases, the binding efficacy of a miRNA. By “binding efficacy” it is meant the ability of a miRNA molecule to bind to a target gene or transcript, and therefore, silence, decrease, reduce, inhibit, or prevent the transcription or translation of the target gene or transcript, respectively. Binding efficacy is determined by the ability of the miRNA to inhibit protein production or inhibit reporter protein production. Alternatively, or in addition, binding efficacy is defined as binding energy and measured in minimum free energy (mfe) (kilocalories/mole).

“Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a primary tumor to a metastatic tumor or to one at risk of developing a metastatic, or from at risk of a primary metastatic event to a secondary metastatic event or from at risk of a developing a primary tumor of one type to developing a one or more primary tumors of a different type. Risk evaluation can also comprise prediction of future clinical parameters, traditional laboratory risk factor values, or other indices of cancer, either in absolute or relative terms in reference to a previously measured population.

An “increased risk” is meant to describe an increased probably that an individual who carries a SNP within BRCA1 will develop at least one of a variety of disorders, such as cancer, compared to an individual who does not carry a the SNP. In certain embodiments, the SNP carrier is 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, or 100× more likely to develop at least one type of cancer than an individual who does not carry the SNP. Moreover, carriers of a SNP within BRCA1 who have developed one cancer are more likely to develop secondary cancers. In certain embodiments, BRCA1 SNP develop at least one secondary cancer 1, 2, 5, 7, 10, 12, 15, 17, 20, 22, 25, 27, or 30 years prior to the average age that a non-carrier develops at least one secondary cancer.

Cell proliferative disorders include a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. Cancers include, but are not limited to, breast and ovarian cancer.

A subject is preferably a mammal. The mammal can be a human, non-human primate, mouse, rat, dog, cat, horse, or cow, but are not limited to these examples. Mammals other than humans can be advantageously used as subjects that represent animal models of a particular disease. A subject can be male or female. A subject can be one who has been previously diagnosed or identified as having a disease and optionally has already undergone, or is undergoing, a therapeutic intervention for the disease. Alternatively, a subject can also be one who has not been previously diagnosed as having the disease. For example, a subject can be one who exhibits one or more risk factors for a disease.

The biological sample can be any tissue or fluid that contains nucleic acids. Various embodiments include paraffin imbedded tissue, frozen tissue, surgical fine needle aspirations, cells of the skin, muscle, lung, head and neck, esophagus, kidney, pancreas, mouth, throat, pharynx, larynx, esophagus, facia, brain, prostate, breast, endometrium, small intestine, blood cells, liver, testes, ovaries, uterus, cervix, colon, stomach, spleen, lymph node, or bone marrow. Other embodiments include fluid samples such as bronchial brushes, bronchial washes, bronchial ravages, peripheral blood lymphocytes, lymph fluid, ascites, serous fluid, pleural effusion, sputum, cerebrospinal fluid, lacrimal fluid, esophageal washes, and stool or urinary specimens such as bladder washing and urine.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For screening individuals for genetic disorders (e.g. prognostic or risk) purposes, if a particular SNP site is found to be useful for screening a disorder, then the skilled artisan would recognize that other SNP sites which are in LD with this SNP site would also be useful for screening the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For screening applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., disease) that is influenced by the causative SNP(s). Thus, polymorphic markers that are in LD with causative polymorphisms are useful as markers, and are particularly useful when the actual causative polymorphism(s) is/are unknown.

Linkage disequilibrium in the human genome is reviewed in: Wall et al., “Haplotype blocks and linkage disequilibrium in the human genome”, Nat Rev Genet. 2003 August; 4(8):587-97; Gamer et al., “On selecting markers for association studies: patterns of linkage disequilibrium between two and three diallelic loci”, Genet Epidemiol. 2003 January; 24(1):57-67; Ardlie et al., “Patterns of linkage disequilibrium in the human genome”, Nat Rev Genet. 2002 April; 3(4):299-309 (erratum in Nat Rev Genet. 2002 July; 3(7):566); and Remm et al., “High-density genotyping and linkage disequilibrium in the human genome using chromosome 22 as a model”; Curr Opin Chem. Biol. 2002 February; 6(1):24-30.

The contribution or association of particular SNPs and/or SNP haplotypes with disease phenotypes, such as cancer, enables the SNPs of the present invention to be used to develop superior tests capable of identifying individuals who express a detectable trait, such as cancer, as the result of a specific genotype, or individuals whose genotype places them at an increased or decreased risk of developing a detectable trait at a subsequent time as compared to individuals who do not have that genotype. As described herein, screening may be based on a single SNP or a group of SNPs. To increase the accuracy of predisposition/risk screening, analysis of the SNPs of the present invention can be combined with that of other polymorphisms or other risk factors of the disease, such as disease symptoms, pathological characteristics, family history, diet, environmental factors or lifestyle factors.

The screening techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders.

### EXAMPLES

### Example 1

### Identification of SNPs in Breast and Ovarian Cancer Associated Genes that could Potentially Modify the Binding Efficacy of MiRNAs

Clinical and molecular classification has successfully clustered breast cancer into subgroups that have biological significance. The categories of subgroups are 1) ER+ and/or PR+ tumors, 2) HER2+ tumors, and 3) triple-negative (TN) tumors (Perou, C. M. et al. Nature 2000. 406, 747-52). The ER+ and/or PR+ and HER2+ tumors together are most prevalent (75%), with the triple negative tumors accounting for approximately 25% of breast cancers. Unfortunately, the triple negative phenotype represents an aggressive and poorly understood subclass of breast cancer that is most prevalent in young, African American women (<40). This subclass has a worse 5-year survival than the other subtypes (72% versus 85%).

DNA was collected from primary tumors in 355 cancer cases and 29 control individuals from Yale for this study. Of these DNA samples, 206 are from the breast. Additionally, 77 ovarian cancer DNA samples, 55 uterine cancer DNA samples, 17 DNA samples were collected from patients that have had breast and ovarian cancer. 29 non-cancerous DNA samples representative of a New Haven, Conn. case control group were also collected. Significant medical information is known for each of these patients participating in this study, such as clinical and pathology information, family history, ethnicity, and survival. The library of samples used in this study has continued to grow.

The BRCA1 gene is associated with increased risk of breast and ovarian cancer and constitutes the focus of this study. The 3′ UTR of BRCA1 was selected according to the University of California Santa Cruz genome browser (publicly available at http://genome.ucsc.edu). The 3′UTR is defined as sequence from the stop codon to the end of the last exon of each gene. Putative miRNA binding sites within the 3′ UTR of the BRCA1 gene were identified by means of specialized algorithms, using the default parameters of each (e.g. PicTar, TargetScan, miRanda, miRNA.org, and MicroInspector). The SNPs residing in miRNA binding sites were identified by searching dbSNP (publicly available at http://www.ncbi.nlm.nih.gov/projects/SNP) and the Ensembl database (publicly available at http://www.ensembl.org/index.html).

PCR amplification of the 3′ UTR of BRCA1 was conducted from DNA cancer samples and cell lines. Ultra high fidelity KOD hot start DNA polymerase (EMD) was used in order to minimize PCR mutation frequency. The thermal cycle program used included one cycle at 95° C. for 2 min, 40 cycles at 95° C. for 20 s, 64° C. for 10 s, and at 72° C. for 40 seconds. Successful PCR amplicons were then sent to the Yale Keck Biotechnology Resource Laboratory (http://keck.med.yale.edu/) for sequencing. The sequences were screened for the presence of both novel and known SNPs. All identified SNPs were recorded.

Once sufficient sequencing results for BRCA1 were obtained, the more time efficient method of high-throughput genotyping was used. Thus, TaqMan PCR assays (Applied Biosystems) were employed, which were designed specifically for the appropriate polymorphisms. The genotyping was preformed using two TaqMan fluorescently labeled probes, one for each allele. Analysis was preformed using the ABI PRISM 7900HT sequence detection system and SDS 2.2 software (Applied Biosystems). The TaqMan reactions were carried out on the cancer samples as well as the global library of DNA samples using the following thermal cycle program: one cycle at 95° C. for 10 min, 50 cycles at 93° C. for 15 seconds, and 60° C. for 1 minute. The assay ID of probes for BRCA1 are as follows:

BRCA1:

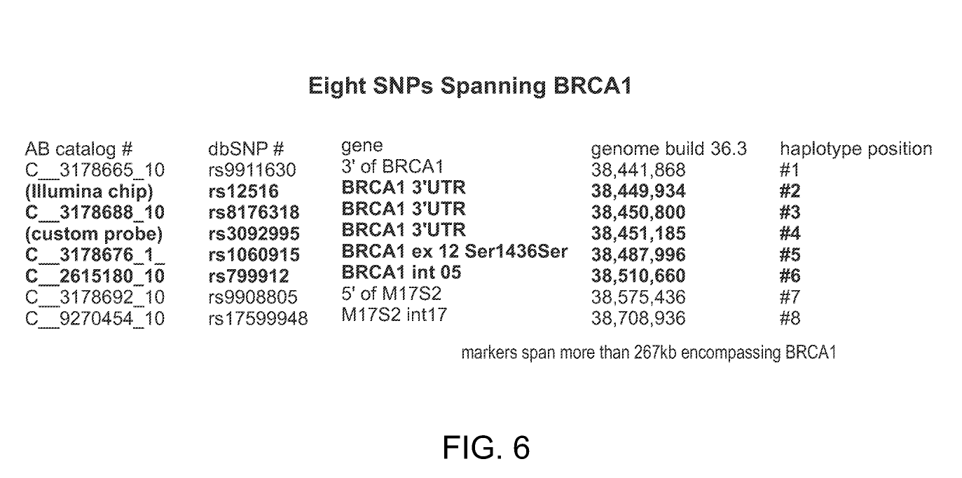

C_{—}3178665_{—}10 (rs9911630),
C_{—}29356_{—}10 (rs12516),
C_{—}3178688_{—}10 (rs8176318),
custom made RS3092995-0001 (rs3092995),
C_{—}3178676_{—}10 rs1060915),
C_{—}2615180_{—}10 (rs799912),
C_{—}3178692_{—}10 (rs9908805),
and C_{—}9270454_{—}10 (rs17599948) (FIG. 6, Table 2).

To preserve DNA samples of study participants, the TaqMan PreAmp Master Mix Kit (Applied Biosystems) was used. The pre-amplification procedure does not amplify the whole genome, but instead we create an “assay pool” consisting of all of the probes of interest. Thus, 18 probes were pooled from 5 different chromosomes and 7 different genes. Over 100 samples were pre-ampled successfully. This method provides a means to pool all of the pertinent probes together and amplify the regions of the genome of interest. The basic protocol is to run preamplification PCR on very low DNA concentrations (results show that reliable results can be gathered from as little as 1.5 ul of 0.5 ng/ul DNA). The preamplification product is then diluted 1:40. The samples are then ready to be used for TaqMan genotyping (procedure described above).

TABLE 2
8 Polymorphisms Studied spanning 267 kb and encompassing BRCA1

| | | | | Genome | Haplotype | | |
|---|---|---|---|---|---|---|---|
| AB Catalog # | dbSNP# | Chromosome | Gene | Build 36.3 | Position | Alleles | Ancestral |
| C_3178665_10 | rs9911630 | 17 | 3′ UTR of | 38,441,868 | #1 | A/G | G |
| | | | BRCA1 | | | | |
| Illumina Chip | rs12516 | 17 | BRCA1 3′UTR | 38,449,934 | #2 | A/G | G |
| C_3178688_10 | rs8176318 | 17 | BRCA1 3′UTR | 38,450,800 | #3 | A/C | C |
| Custom Probe | rs3092995 | 17 | BRCA1 3′UTR | 38,451,185 | #4 | C/G | C |
| C_3178676_10 | rs1060915 | 17 | BRCA1 ex 12, | 38,487,996 | #5 | A/G | A |
| | | | S1436S | | | | |
| C_2615180_10 | rs799912 | 17 | BRCA1 int 5 | 38,510,660 | #6 | C/T | C |
| C_3178692_10 | rs9908805 | 17 | 5′ of BRR1 | 38,575,436 | #7 | C/T | T |
| C_9270454_10 | rs17599948 | 17 | NBR1 int 17 | 38,708,936 | #8 | A/G | A |

Bolded polymorphisms comprise the optimum set of SNPs required to predict a subject's risk of developing breast cancer.
The 8 SNPs spanning 2 genes and about 267 kb were studied using Taqman SNP genotyping assays.

TABLE 3
Study Population

| | | | | | | | |
|---|---|---|---|---|---|---|---|
| | | | ER+/ | Ovar- | Uter- | Breast/ | Yale |
| TN | MP | HER2+ | PR+ | ian | ine | Ovarian | Controls |

BRCA1 (Total = 384)

| | | | | | | | | |
|---|---|---|---|---|---|---|---|---|
| Sequenced | 7 | 0 | 18 | 14 | 43 | 34 | 8 | 14 |
| Genotyped* | 76 | 39 | 47 | 44 | 77 | 55 | 17 | 29 |

*Numbers represent the number of patients genotyped for 8 different SNPs.
In BRCA1, all patients that were sequenced were then also genotyped.
Numbers of samples able to be directly sequenced for some subtypes, especially MP, are limited due to many or all of the samples being FFPE.

### Example 2

### Evaluation of Sequence Variations in miRNA Complementary Sites within BRCA1 Using Tissue from Breast and Ovarian Tumors, Adjacent Normal Tissue and Normal Tissue Samples

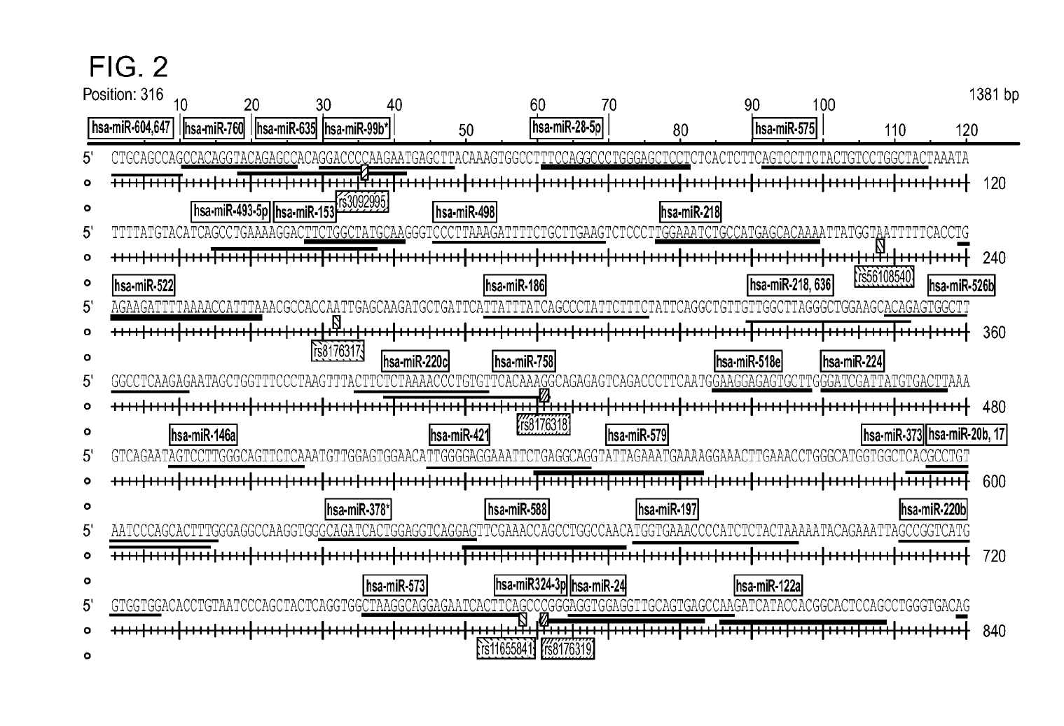

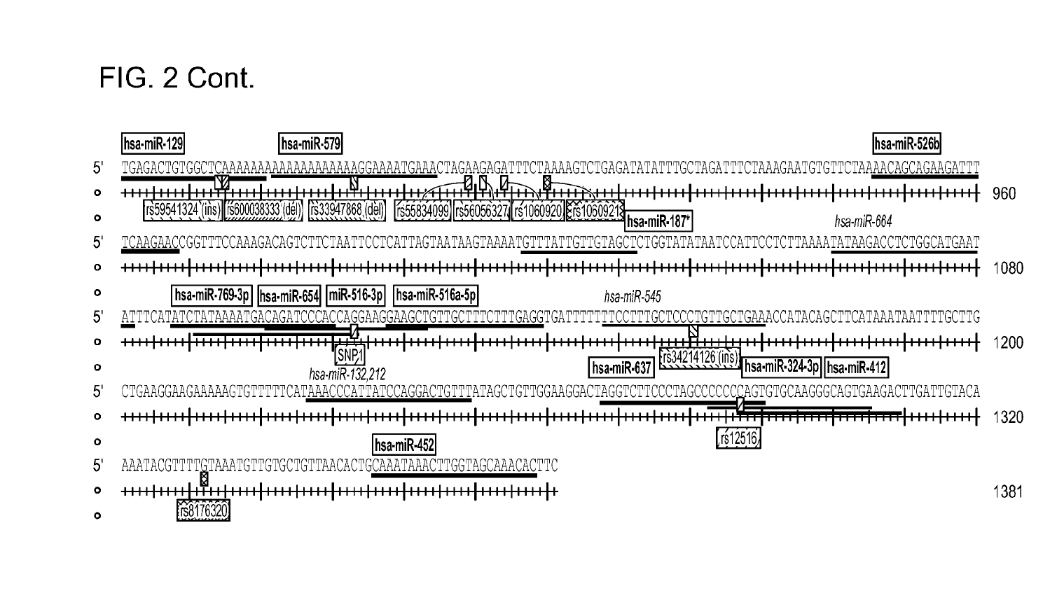

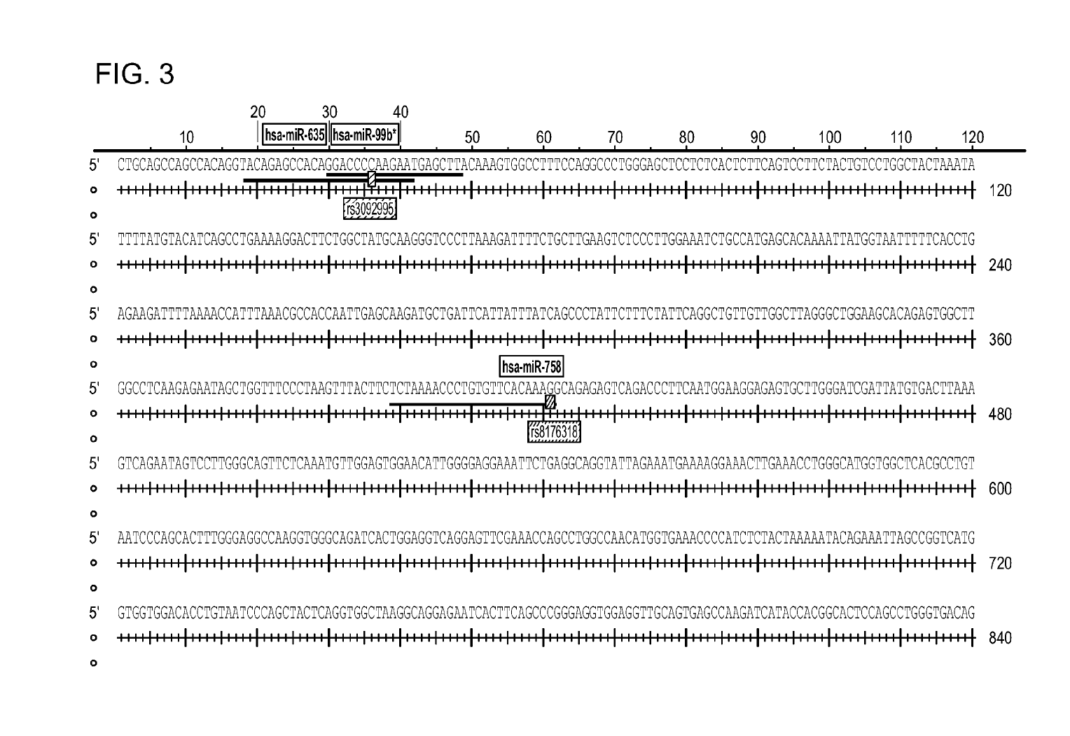

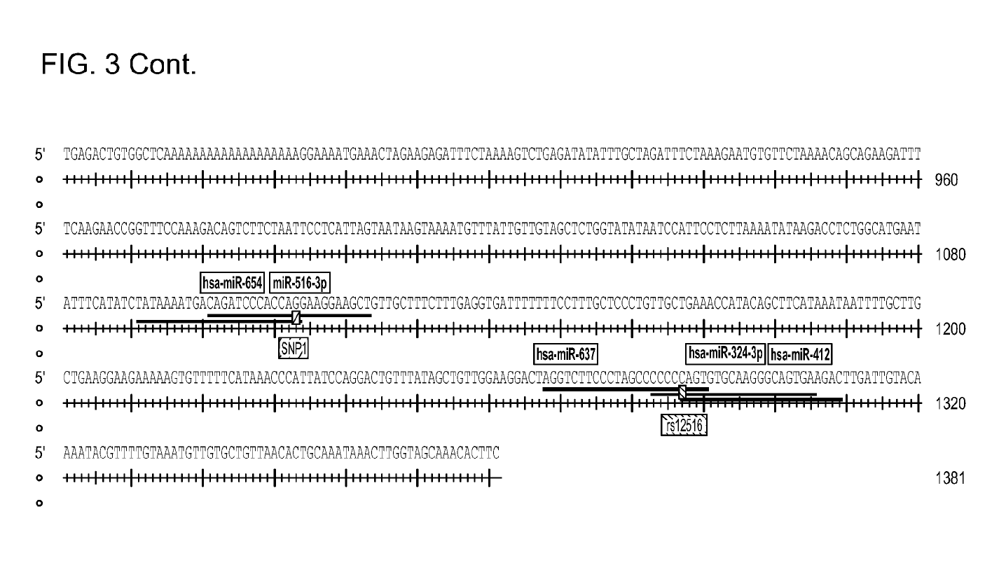

BRCA1 has a highly conserved 3′ UTR of 1381 nucleotides. The 3′ UTR has 16 known SNPs. Nine of these SNPs are located in predicted miRNA binding sites and 4 of these 9 are located in predicted seed region binding sites. However, among these 16 SNPs, only 3 SNPs (rs3092995, rs8176318, and rs12516) have been found in the sequenced DNA samples thus far. Additionally, one novel SNP (SNP1) has been identified that resides in a predicted miRNA binding sites. Of note, this SNP has only been found in one patient, both in tumor and adjacent normal tissue. The results are reproducible (FIGS. 2 and 3). Of the four SNPs that have both been identified by sequencing and have predicted miRNA binding sites, two of these (rs3092995 and rs12516) are located in the seed regions of predicted miRNA binding sites (FIG. 3). None of the SNPs we have identified are located in highly conserved predicted miRNA binding sites.

More specifically, rs3092995 is located where the following two poorly conserved miRNAs are predicted to bind: hsa-miR-99b and has-miR-635. Rs3092995 is predicted to lie in the seed region of has-miR635. Rs8176318 is located where hsa-miR-758 is predicted to bind. SNP1 is located where both hsa-miR-654 and hsa-miR-516-3p are predicted to bind. Lastly, rs12516 is located where hsa-miR-637, hsa-miR-324-3p, and hsa-miR-412 are predicted to bind. Rs12516 falls in the predicted seed region of hsa-miR-637 (FIG. 3).

Once the BRCA1 3′UTR was mapped in the study cancer populations, a more high-throughput method of genotyping the cancer DNA samples was used. To accomplish this, the TaqMan PCR assays (Applied Biosystems) were used, which were designed specifically for the 3 main SNPs located through sequencing our cancer populations. Genotyping was preformed using two TaqMan fluorescently labeled probes, one for each allele. Analysis was preformed using the ABI PRISM 7900HT sequence detection system and SDS 2.2 software (Applied Biosystems). The TaqMan reactions were carried out on our cancer samples as well as the global DNA samples.

### Example 3

### Prevalence of BRCA1 SNPs in Local Versus Global Populations

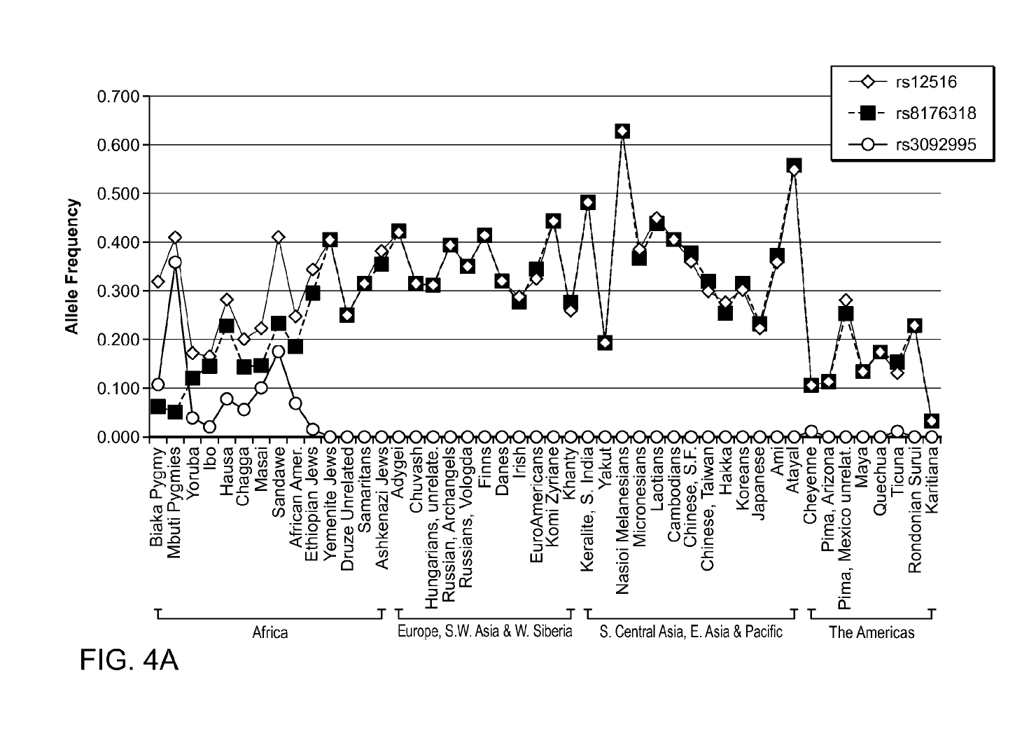

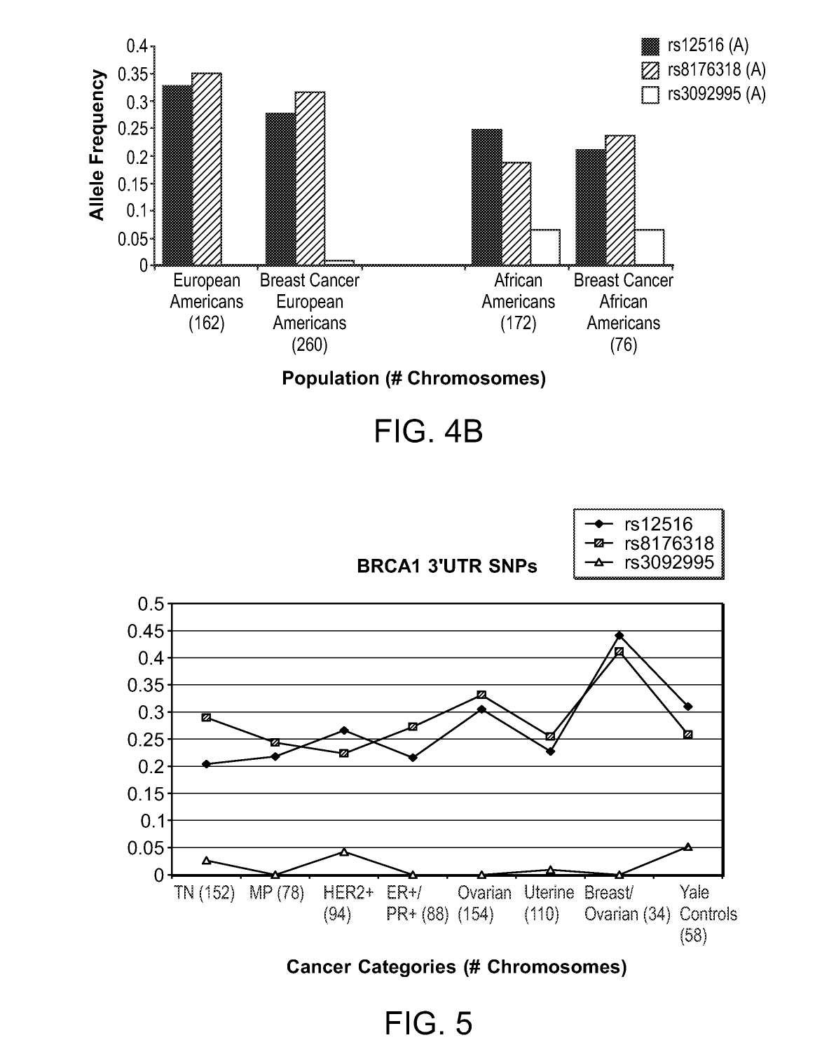

FIG. 4 shows the genotyping results for BRCA1 3′UTR from the global library of 46 World populations, including 2,472 individuals. As shown in FIG. 4, rs8176318 and rs12516 are almost always inherited together in the general population. Excluding the African ethnicities they are found in 31.6 and 31.7% of the population respectively. Additionally, rs3092995 is extremely rare through most of the World. Excluding African ethnicities, rs3092995 is on average not found in the population. These two interesting trends do not hold true for the African populations however. Within the African populations (There are 10, From the far left of the chart, Biaka Pygmy to Ethiopian Jews), rs3092995 is found in 10.2% of the populations and rs8176318 and rs12516 are at a decreased likelihood of being inherited together. It appears that when rs8176318 and rs12516 are not inherited together, rs12516 is always at a higher prevalence than rs8176318 (27.8% and 16.3% respectively).

Concurrently, 384 individuals were analyzed from 7 cancer populations and 1 population of Yale controls for the same three SNPs in the BRCA1 3′UTR (FIG. 5). Interestingly, the trend observed in the World populations (FIG. 4) is not mirrored in the study cancer populations. However, there are a few similarities. For example, rs3092995 is found at a rate of 1.6% of the study cancer populations and the Yale control group. Also, within the Yale control group, rs8176318 and rs12516 display the same trend as in the non-African World populations. That is, within the non-African World populations these 2 SNPs are present in about 31% of the population and within our Yale cohort, they are present in 28% of the population, usually being inherited together. However, there is a striking difference observed in the various cancer populations rs8176318 and rs12516 are less likely to be inherited together. This trend is similar to what is found in the African populations. However, what makes this trend even more interesting is that within the African populations SNP rs12516 is at a higher frequency in the populations than rs8176318 (27.8% and 16.3%, respectively). But, in the study cancer populations rs8176318 is at a higher frequency than rs12516 in our breast cancer populations (excluding HER2+) (26.9% and 21.3%, respectively).

In response to the previous results, this region of chromosome 17 was saturated with more informative SNPs. Our reasoning was two-fold, to solve the lineage evolution of the region and to run haplotype analysis. To accomplish this, 5 additional informative SNPs were added that encompass BRCA1 (FIG. 6). These SNPs are ordered from the bottom of the chromosome, up (3′ to 5′) because BRCA1 is on the reverse strand. These 8 SNPs span 2 genes (BRCA1 and NBR1) and about 267 kb. This large chromosomal region allows for us to observe genetic variability despite the strong linkage disequilibrium observed for haplotype analysis (Gu, S., Pakstis, A. J. and Kidd, K. K. Bioinformatics 2005. 21, 3938-9). Haplotype analysis is a powerful way to analyze affects of SNPs in genes of interest. The theory behind conducting haplotype analysis is: If the disease gene has undergone negative selective pressure, the linked variation in the disease-carrying chromosome may be at lower frequency within the population.

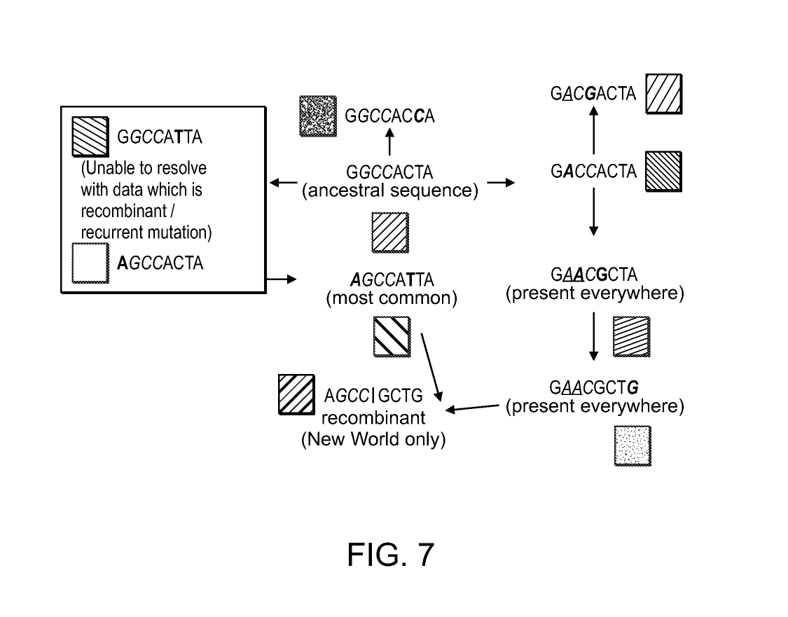

The evolution of these 8 SNPs spanning BRCA1 was determined (FIG. 7). In FIG. 6 each SNP is assigned a haplotype position (1-8). These positions correlate to the “fake” haplotypes observed in FIG. 7. For example, the ancestral sequence is eight letters “GGCCACTA (SEQ ID NO: 8),” each letter (from left to right) correlates to the numbered position. To determine the ancestral states of the SNPs, the same TaqMan assays that are used on our human samples were employed, however, these assays were used to genotype genomic DNA from non-human primates. The ten most common haplotypes can be explained by accumulation of variation on the ancestral haplotype. Most of the directly observed haplotypes can be ordered, differing by one derived nucleotide change. More specifically, in FIG. 7, the two haplotypes that are boxed were unresolved regarding which occurred first in the lineage with the SNPs that were employed. The AGCCATTA (SEQ ID NO: 2) haplotype is currently the most commonly observed haplotype in the World. Two haplotypes, GAACGCTA (SEQ ID NO: 3) and GAACGCTG (SEQ ID NO: 4), are present in all regions of the World. The AGCC-GCTG (SEQ ID NO: 19) haplotype is found in the new world only, which indicates regions of South, Central and North America (For complete descriptions of populations go to ALFRED: http://alfred.med.yale.edu/).

Haplotype prevalences between the global populations and the study cancer populations were compared. This comparison revealed significant differences between the haplotypes observed between the two groups, as well as one or more haplotypes that are associated with increased risk to breast and/or ovarian cancer.

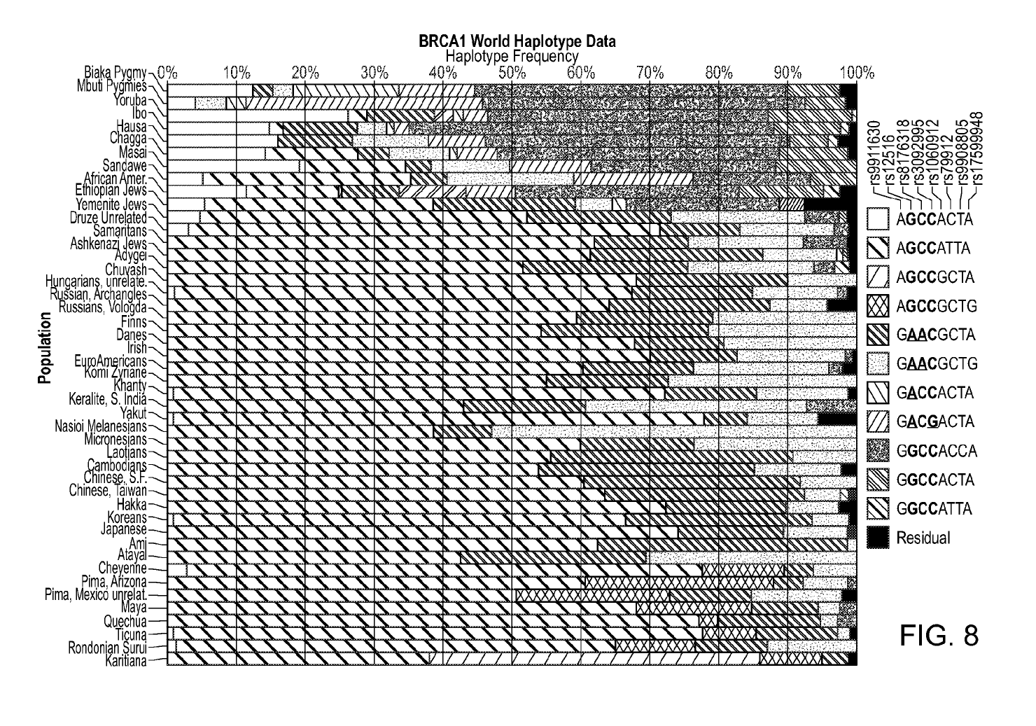

The eight SNPs in the 46 World populations that include 2,472 individuals (FIG. 8) were genotyped. The haplotype data in FIG. 8 was expected based on the haplotype evolution data. More specifically, the observed ancestral haplotype, GGCCACTA (SEQ ID NO: 8), was only found in African ethnicities. The most common haplotype, AGCCATTA (SEQ ID NO: 2), was found at high levels throughout the World. Two haplotypes, GAACGCTA (SEQ ID NO: 3) and GAACGCTG (SEQ ID NO: 4), were again found throughout the World. The recombinant haplotype, AGCC-GCTG (SEQ ID NO: 19), (as was predicted by haplotype evolution) was in fact found in the New World only. This chart is reminiscent of the patterns found when the BRCA1 3′UTR is genotyped (FIG. 4). As was noted when discussing FIG. 4, the African populations depict a very different pattern. This observation again holds true here. In FIG. 8, the first 10 ethnicities are of African descent (Biaka Pygmy to Ethiopian Jews) and display a unique haplotype pattern. For example, the following haplotypes, GGCCACCA (SEQ ID NO: 7), GACGACTA (SEQ ID NO: 5), GACCACTA (SEQ ID NO: 20), and AGCCACTA (SEQ ID NO: 1) are all unique to Africans. Lastly, the sequence labeled “residual” most likely represents multiple haplotypes at rare frequency in the population. The 46 populations range in size from as few as 26 individuals (Masia) to as many as 222 individuals (Laotians). Each population averages to have 96.6 individuals represented.

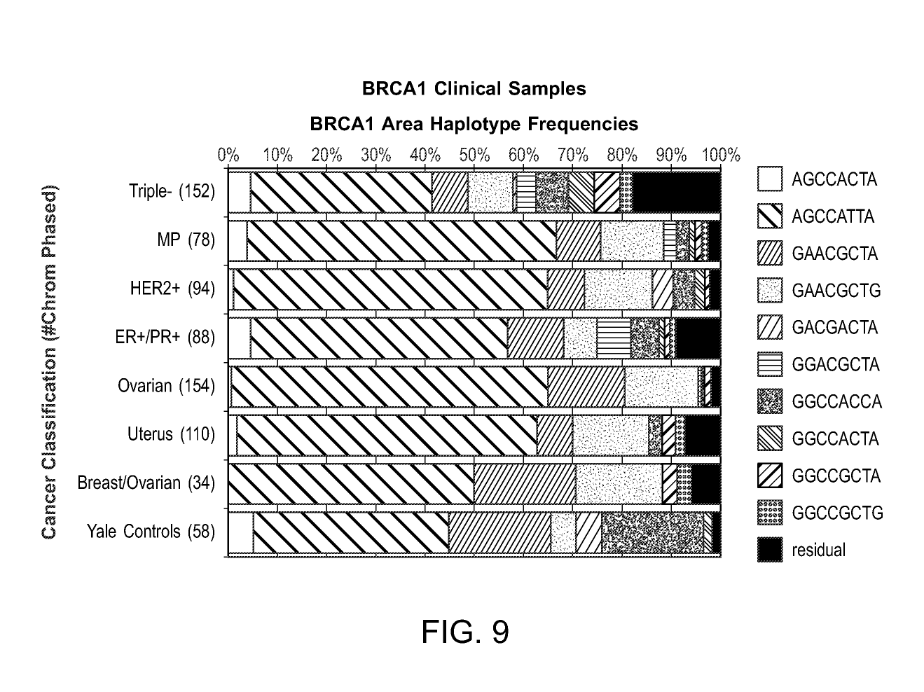

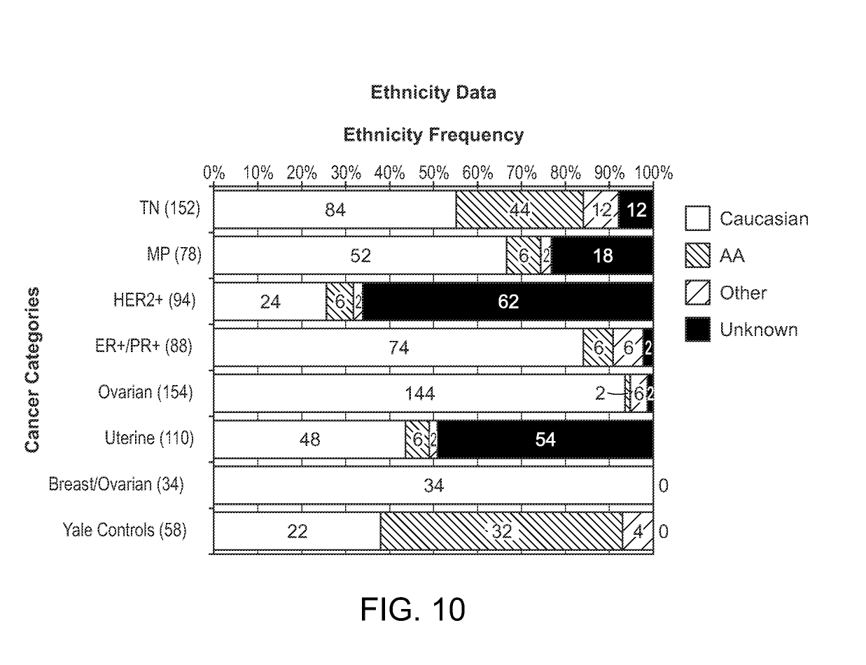

FIG. 9 shows our haplotype data from 7 cancer populations and 1 Yale control group totaling 384 individuals. Importantly, regarding a comparison of the general World haplotype trends with FIG. 9, many of the same haplotypes were observed. For example, the AGCCATTA (SEQ ID NO: 2) haplotype was still the most commonly observed. Additionally, two haplotypes, GAACGCTA (SEQ ID NO: 3) and GAACGCTG (SEQ ID NO: 4), were found throughout the World and also found throughout the populations represented in FIG. 9. The GGCCACCA (SEQ ID NO: 7) haplotype that was common among African populations in FIG. 8 was frequently observed also in FIG. 9. This may be because there are African Americans in all of the populations that the GGCCACCA (SEQ ID NO: 7) haplotype was observed. The only population in FIG. 9 that the GGCCACCA (SEQ ID NO: 7) haplotype was not observed was the breast/ovarian population and this group was only made up of Caucasians (See FIG. 10 for ethnicity data). However, strikingly, the haplotypes observed within the TN subtype of breast cancer varied quite significantly from not only the World populations, but also the other cancer populations and our Yale control group (FIG. 9). There are 3 haplotypes that are particularly interesting. These haplotypes are GGACGCTA (SEQ ID NO: 6), GGCCGCTA (SEQ ID NO: 9), and GGCCGCTG (SEQ ID NO: 10) (FIG. 9 and Table 4). These 3 unique haplotypes made up 12% of the haplotypes observed in the TN cancer group and were not represented in the World haplotypes (except possibly in residual). The GGCCGCTA (SEQ ID NO: 9) haplotype is of particular interest because it is found in all 7 cancer groups. Additionally, the TN breast cancer group has the largest proportion of residual haplotypes making up almost 18% of the haplotypes (FIG. 9). The criteria for residual haplotypes is <1% of all samples across all categories. Within the TN residuals is a haplotype “GGACGCTG” (SEQ ID NO: 21). This haplotype makes up 4% of the TN haplotypes. It is however classified as residual because it is rarely observed in other categories (it is observed once in ovarian and once in uterine cancer groups). Table 4 shows a closer analysis of affected SNPs within these unique and interesting haplotypes. The Ancestral haplotype, GGCCACTA (SEQ ID NO: 8), and the most common haplotype, AGCCATTA (SEQ ID NO: 2), are depicted for comparison purposes. SNPs rs8176318, rs1060915, and rs17599948 are exemplary sites of variation resulting in the unique haplotypes. Rs8176318 is significant because it is located in the 3′UTR of BRCA1 and also located in predicted miRNA binding sites. Rs1060915 is also significant because it is located in exon 12 of the coding region of BRCA1. Coding regions are also sites of target for miRNAs.

TABLE 4

| | | | *** | | *** | | | *** |
|---|---|---|---|---|---|---|---|---|
| | rs9911630 | rs12516 | rs8176318 | rs3092995 | rs1060915 | rs799912 | rs9908805 | rs17599948 |
| dbSNP# | 3′ UTR of | 3′ UTR of | 3′ UTR of | 3′ UTR of | BRCA1 ex 12 | BRCA1 | 5′ UTR ofNBR1 | NBR1 |
| Gene | BRCA1 | BRCA1 | BRCA1 | BRCA1 | Ser1436SER | int. #5 | (a/k/a M17S2) | int. #17 |
| Alleles | G/A | G/A | C/A | C/G | A/G | C/T | T/C | A/G |
| Ancestral | G | G | C | C | A | C | T | A |
| (SEQ ID NO: 8) | | | | | | | | |
| Most Common | A | G | C | C | A | T | T | A |
| (SEQ ID NO: 2) | | | | | | | | |
| (SEQ ID NO: 3) | G | G | A | C | G | C | T | A |
| (SEQ ID NO: 9) | G | G | C | C | G | C | T | A |
| (SEQ ID NO: 10) | G | G | C | C | G | C | T | G |
| Found in Residual | G | G | A | C | G | C | T | G |
| (SEQ ID NO: 21) | | | | | | | | |

Underlined dbSNP#s represent essential sites of polymorphism for predicting risk of developing breast or ovarian cancer.
rs1060915 SNP: When the variant allele (A) is homozygous, and the effects of this mutation are studied in distinct ethnic groups, the association of breast cancer in African Americans versus Controls is statistically significant (p = 0.01). When the association is further refined to triple negative (TN) breast cancer in African Americans versus Controls, the results are more significant (p = 0.005).

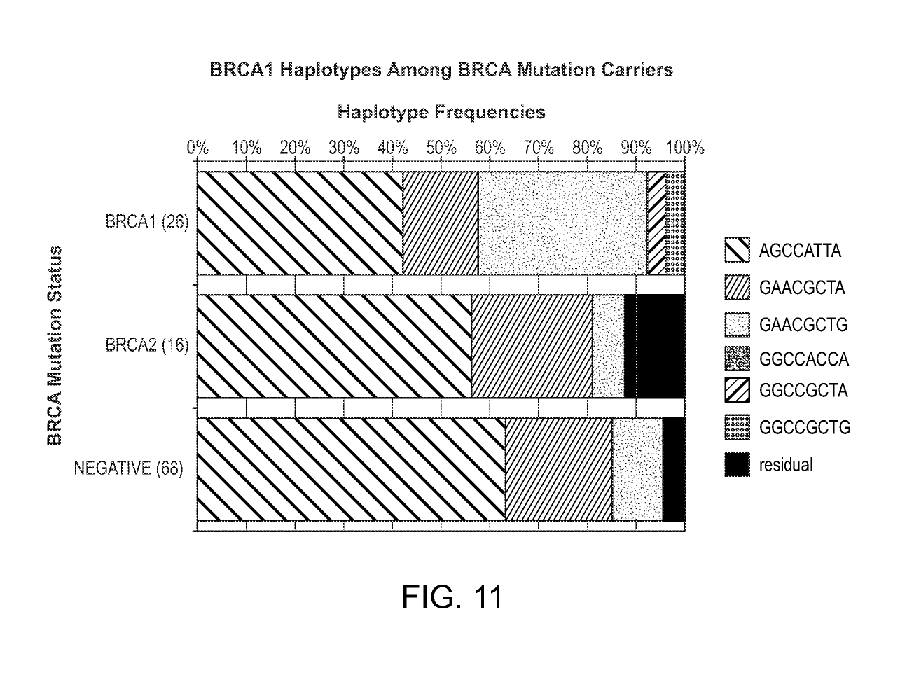

To further analyze these cancer groups, the SNP data was correlated to other known TN breast cancer risk factors. FIG. 11 is a representation of the BRCA1 haplotype data by coding region mutation status. In this study, 110 patients have been BRCA1 tested and analyzed by haplotype. BRCA1 mutations are common in TN breast cancer, so it was expected that two of the unique haplotypes, GGCCGCTA (SEQ ID NO: 9) and GGCCGCTG (SEQ ID NO: 10), were found among BRCA1 mutation carriers making up 8% of the population.

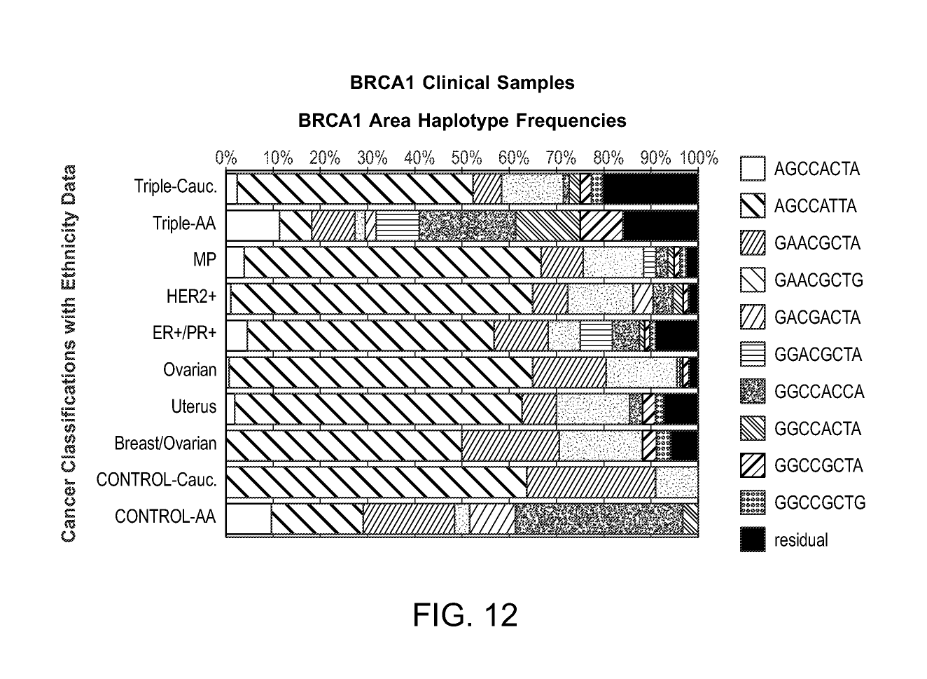

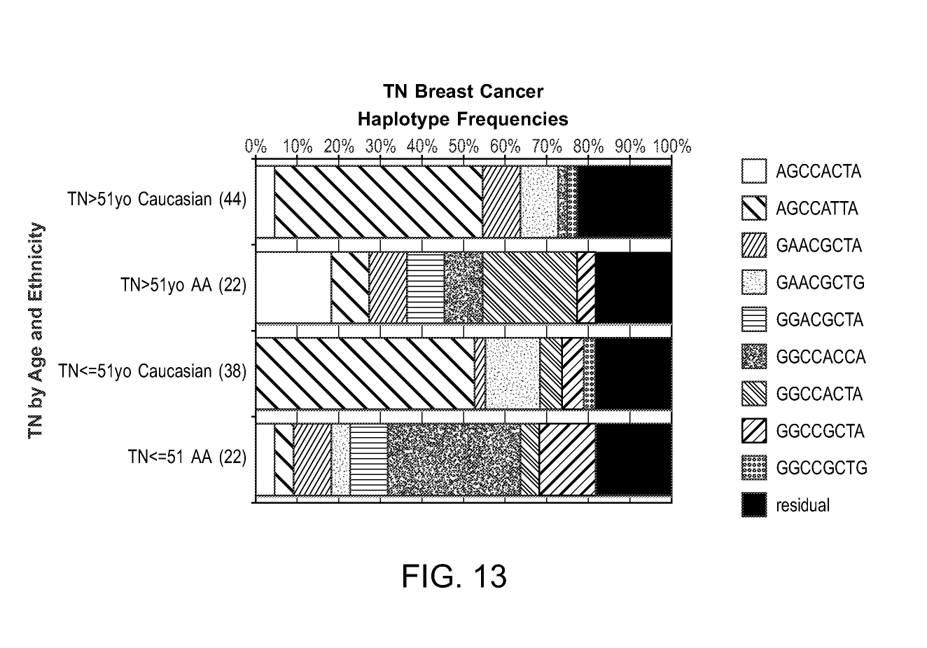

FIGS. 12 and 13 were made to confirm that TN breast cancers have a unique SNP signature and not as result of the diversity of the African populations. FIG. 12 confirms that in fact when the Yale control and TN groups were compared by African American and Caucasian ethnicities, the TN African Americans were different from both control ethnicities and TN Caucasians. In particular the GGACGCTA (SEQ ID NO: 6) and GGCCGCTA (SEQ ID NO: 9), haplotypes are prevalent in TN African Americans. This was expected because TN breast cancer is most prevalent among young African American women, i.e. <40 years old (yo), and is interesting. In FIG. 13, the differing ethnicities were further compared by age within Yale Controls and TN breast cancer groups. When compared by age, it is clear that the GGACGCTA (SEQ ID NO: 6) haplotype was only found within the African American populations, the GGCCGCTA (SEQ ID NO: 9) haplotype was confined to Caucasians. The GGCCGCTA (SEQ ID NO: 9) haplotype was found mostly in the young populations (<=51 yo), however it was also found in older African Americans. Lastly, within the TN African American (AA) populations, the ancestral haplotye is significantly more prevalent in the older group of TN AA. In the younger TN AA group the GGCCACCA (SEQ ID NO: 7) haplotype is more prevalent. This makes sense with the lineage data (FIG. 7).

### Example 4

### Rare BRCA1 Haplotypes Associated with Breast Cancer Risk

Genetic markers that identify women at an increased risk of developing breast cancer exist, yet the majority of inherited risk remains elusive. While numerous BRCA1 coding sequence mutations are associated with breast cancer risk, mutations in BRCA1 polymorphisms disrupting microRNA (miRNA) binding can be functional and can act as genetic markers of cancer risk. Therefore, the hypothesis was tested that such polymorphisms in the 3′UTR of BRCA1 and haplotypes containing these functional polymorphisms may be associated with breast cancer risk. Through sequencing and genotyping three 3′UTR variants were identified in BRCA1 that are polymorphic in breast cancer populations, one of which (rs8176318, variant allele A in homozygosity), shows significant cancer association for African American women and specifically predicts for the risk of developing triple negative breast cancer for African American women (p=0.04 and p=0.02, respectively). Through haplotype analysis it was discovered that breast cancer patients (n=221) harbor five rare haplotypes, including these 3′UTRs variants that are not commonly found in control populations (9.50% for all breast cancer chromosomes and 0.11% for control chromosomes, p=0.0001). Three of the five rare haplotypes contain the rs8176318 BRCA1 3′UTR functional allele. Furthermore, these haplotypes are not biomarkers for BRCA1 coding region mutations, as they are found rarely in BRCA1 mutant breast cancer patients (1/129=0.78%; 1/129 patients, or 1/258 chromosomes). These rare BRCA1 haplotypes represent new genetic markers of increased breast cancer risk.

### Materials and Methods

### Study Populations

After approval from the Human Investigation Committee at Yale, samples from patients with breast cancer receiving treatment at Yale/New Haven Hospital (New Haven, Conn.) were collected from a total of 221 consenting individuals and samples consisted of 180 tumor FFPE and 41 germline DNA sources (81.4%, and 18.6%, respectively) on HIC protocol #0805003789. Germline DNA samples were collected from 22 blood and 19 saliva sources (53.7% and 46.3%, respectively). Patient data were collected including age, ethnicity and family history of cancer. Breast cancer subtypes were established by pathologic classification. Controls were recruited from Yale/New Haven Hospital and included people without any personal history of cancer except non-melanoma skin cancer. All samples were saliva samples. Information including age, ethnicity and family history was recorded. For BRCA1 3′UTR analysis of genotype and cancer association 194 germline DNA controls were used (92 European Americans and 102 African Americans) and 205 tumor FFPE and germline DNA samples from breast cancer patients with known tumor subtype and ethnicity. 129 unrelated BRCA1 mutation carriers were ascertained at Erasmus University Medical Center through the Rotterdam Family Cancer Clinic and DNA was isolated from peripheral blood samples as described below.

For global populations, we used our resource at Yale University of 2,250 unrelated individuals representing 46 populations from around the world. This resource is well documented among genetic studies (Chin L J, et al. Cancer research 2008; 68(20):8535-40; Speed W C, et al. The pharmacogenomics journal 2009; 9(4):283-90; Speed W C, et al. Am J Med Genet B Neuropsychiatr Genet. 2008; 147B(4):463-6; Yamtich J, et al. DNA repair 2009; 8(5):579-84.). The 46 populations represented in this study include 10 African (Biaka Pygmy, Mbuti Pygmies, Yoruba, Ibo, Hausa, Chagga, Masai, Sandawe, African Americans, and Ethiopian Jews), 3 Southwest Asian (Yemenite Jews, Druze and Samaritans), 10 European (Ashkenazi Jews, Adygei, Chuvash, Hungarians, Archangel Russians, Vologda Russians, Finns, Danes, Irish and European Americans), 2 Northwest Asian (Komi Zyriane and Khanty), 1 South Asian (S. Indian Keralite), 1 Northeast Siberian (Yakut), 2 from Pacific Islands (Nasioi Melanesians and Micronesians), 9 East Asian (Laotians, Cambodians, Chinese from San Francisco, Taiwan Han Chinese, Hakka, Koreans, Japanese, Ami and Atayal), 4 North American (Cheyenne, Pima from Arizona, Pima from Mexico, Maya) and 4 South American (Quechua, Ticuna, Rondonia Surui, Karitiana). All subjects gave informed consent under protocols approved by the committees governing human subjects research relevant to each of the population samples. Sample descriptions and sample sizes can be found in the Allele Frequency Database by searching for the population names (http://alfred.med.yale.edu) and in a previous publication (Cheung K H, et al. Nucleic acids research 2000; 28(1):361-3). DNA samples were extracted from lymphoblastoid cell lines established and/or grown. The methods of transformation, cell culture, and DNA purification have been described (Anderson M A and Gusella J F. In vitro 1984; 20(11):856-8). All volunteers were apparently normal and otherwise healthy adult males or females and samples were collected after receipt of appropriate informed consent under protocols approved by all relevant institutional review boards.

### Evaluation of 3′ UTR Sequences

DNA was isolated from frozen and FFPE tumor breast tissue using RecoverAll Total Nucleic Acid Isolation Kit (Ambion), and from blood and saliva using the DNeasy Blood and Tissue kit (Qiagen). The whole 3′UTR of BRCA1 was amplified using KOD Hot Start DNA polymerase (Novagen) and DNA primers specific to this sequence: BRCA1: 5′-GAGCTGGACACCTACCTGAT-3′ (SEQ ID NO: 22) and 5′-GAGAAAGTCGGCTGGCCTA-3′ (SEQ ID NO: 23). PCR products were purified using the QIAquick PCR purification kit 161 (Quiagen) and sequenced using nested primers:

BRCA1:
(SEQ ID NO: 24)
5′-CCTACCTGATACCCCAGATC-3′
and
(SEQ ID NO: 25)
5′-GGCCTAAGTCTCAAGAACAGTC-3′.

### Marker Typing

For high throughput genotyping, TaqMan 5′ nuclease assays (Applied Biosystems) were designed specifically to identify alleles at each SNP location. We determined the ancestral states of the 8 SNPs employed by using the same TaqMan assays to genotype genomic DNA for non-human primates-3 bonobos (Pan paniscus), 3 chimpanzees (Pan troglodytes), 3 gibbons (Hylobates), 3 gorillas (Gorilla gorilla), and 3 orangutans (Pongo pygmaeus).

### Statistics

Frequencies of genotypes across populations were compared using Chi-Square Test of Association and Fisher Exact probability test. Significance of haplotype data was evaluated using Chi-Square Test of Association. P values were considered statistically significant if p>0.05. All sites within the haplotype are in accordance with Hardy-Weinberg equilibrium among controls within each ethnic group. We used PHASE (software for haplotype reconstruction and recombination rate estimation from population data) to infer haplotypes of patients and control individuals (30, 31) without subpopulation information. PHASE software provides estimates of the certainty of haplotype assignment. In view of the fairly simple haplotype structure of the BRCA1 gene, the PHASE algorithm was extremely accurate. Of the haplotypes that did need to be estimated, PHASE estimated our cohort with 99% certainty.

### Results

### Identifying SNPs in the BRCA1 3′UTR

There are numerous known BRCA1 3′UTR SNPs (Table 5). To identify the frequency of these known polymorphisms and/or to identify novel SNPs in breast cancer patients, we sequenced the entire 3′UTR of BRCA1 in breast cancer patients with the three known breast cancer subtypes (TN=7, HER2+=18, and ER/PR+/HER2−=14). The initial screen of the entire BRCA1 3′UTR in these patients identified variation at only the three previously reported functional SNPs: rs12516, rs8176318, and rs3092995 (Table 5). Additionally, we identified a novel SNP in the BRCA1 3′UTR. The novel SNP in BRCA1 is 6824G/A or 5711+1113G/A. This SNP was identified as heterozygous in a 61 year old African American HER2+ patient for the previously unseen A allele. To better evaluate the frequency of these variants across populations we performed population specific genotyping in 2,250 non-cancerous individuals making up 46 populations worldwide (FIG. 4A). The three identified BRCA1 3′UTR SNPs, rs12516, rs8176318, and rs3092995 are in strong linkage disequilibrium in populations and vary by ethnicity.

TABLE 5
Known BRCA1 3′UTR polymorphisms.

| | | | Cbr:bp dbSNP | Al- | Ancestral | |
|---|---|---|---|---|---|---|
| Gene | ID | Type | build 130 | leles | Allele | Class |
| BRCA1 | Rs3092995* | 3′UTR | 17:38451185 | C/G | C | SNP |
| | 56108540 | 3′UTR | 17:38450993 | G/A | A | SNP |
| | Rs8176317 | 3′UTR | 17:38450949 | A/G | A | SNP |
| | Rs8176318* | 3′UTR | 17:38450800 | G/T | G | SNP |
| | Rs11655841 | 3′UTR | 17:38450443 | C/G | G | SNP |
| | Rs8176319 | 3′UTR | 17:38450440 | C/T | C | SNP |
| | Rs59541324 | 3′UTR | 17.38450367-6 | —/Aᵤ | Complex | STRP |
| | Rs60038333 | | 17:38450366 | | | |
| | Rs68017638 | | 17:38450365-6 | | | |
| | Rs33947868 | | 17:38450348-9 | | | |
| | Rs55834099 | 3′UTR | 17:38450332 | G/A | A | SNP |
| | Rs56056327 | 3′UTR | 17:38450331 | G/A | G | SNP |
| | Rs1060920 | 3′UTR | 17:38450327 | A/G | A | SNP |
| | Rs1060921 | 3′UTR | 17:38450321 | A/T | A | SNP |
| | Rs34214126 | 3′UTR | 17:38450061-0 | —/C | — | Inser- |
| | | | | | | tion |
| | Rs12516* | 3′UTR | 17:38449934 | C/T | C | SNP |
| | Rs8176320 | 3′UTR | 17:38449889 | A/G | G | SNP |

List of known BRCA1 3′UTR SNPs presented on the coding strand. Locations of polymorphisms are based on dbSNP build 130.
*The three SNPs studied.
†These are variants in a poly A. We have classified them as STRP, or short tandem repeat polymorphisms.
Based on chimpanzee, orangatan, and human reference sequences, the STRP is complex: A₁₆₋₁₉G₂A₃₋₄.

TABLE 6
BRCA1 3′UTR Sequencing Results

| | | Genotype | | | |
|---|---|---|---|---|---|
| | | G/G-C/C- | A/G-A/C- | A/A-A/A- | G/G-A/C- |
| Population | | C/C | C/C | C/C | G/C |
| BRCA1 | Triple | 5 (71.4%) | 0 | 2 (28.6%) | 0 |
| | Negative | | | | |
| | (7) | | | | |
| | European | 4 | 0 | 1 | |
| | Americans | | | | |
| | (5) | | | | |
| | African | 1 | 0 | 0 | 0 |
| | Americans | | | | |
| | (1) | | | | |
| | Other (1) | 0 | 0 | 1 | 0 |
| | HER2+ (18) | 14 (77.8%) | 0 | 2 (11.1%) | 2 (11.1%) |
| | European | 9 | 0 | 1 | 0 |
| | Americans | | | | |
| | (10) | | | | |
| | African | 0 | 0 | 0 | 2 |
| | Americans | | | | |
| | (2) | | | | |
| | Unknown (6) | 5 | 0 | 1 | 0 |
| | ER/PR+ (14) | 12 (85.7%) | 2 (14.3%) | 0 | 0 |
| | European | 8 | 1 | 0 | 0 |
| | Americans | | | | |
| | (9) | | | | |
| | African | 1 | 0 | 0 | 0 |
| | Americans | | | | |
| | (1) | | | | |
| | Other (2) | 2 | | 0 | 0 |
| | Unknown (2) | 1 | 1 | 0 | 0 |
| | Total (39) | 31 (79.5%) | 2 (5.1%) | 4 (10.3%) | 2 (5.1%) |

The entire BRCA1 3′UTR was sequenced from 39 breast cancer patients. The genotypes observed were G/G-C/C-C/C, A/G-A/C-C/C, A/A-A/A-C/C, and G/G-A/C-G/C. The positions are rs12516, rs8176318, and rs3092995, respectively. Allele A is the derived allele at positions rs12516 and rs8176318. Allele G is the derived allele at rs3092995.

Since significant variation was observed in the identified 3′UTR SNPs by ethnicity in the control populations, the variation of these SNPs in breast cancer patients of different ethnicity was subsequently determined. These SNPs were genotyped in 130 breast cancer European American patients and 38 breast cancer African American patients and variation was observed across these groups (FIG. 4B). To determine the association of these SNPs with tumor risk, the frequency of these SNPs between breast cancer patients and ethnicity matched controls was compared. It was determined that the rare variant at rs8176318 in the homozygous form (A/A) is 207 significantly associated with breast cancer for African Americans [Odds ratio (OR), 9.48; 95% confidence interval (CI), 1.01-88.80; p=0.04]. No tumor association was observed between breast cancer European Americans and the rs8176318 SNP (Table 7).

TABLE 7
The BRCA1 3′UTR SNP rs8176318 and breast cancer association by ethnicity and breast cancer subtype

| | | BC EA (165) vs | | BC AA (40) vs | | TN EA (66) vs | | TN AA (31) vs | |
|---|---|---|---|---|---|---|---|---|---|
| | | Control EA (92) | | Control AA (102) | | Control EA (92) | | Control AA (102) | |
| | | OR | P | OR | P | OR | P | OR | P |
| SNP (gene) | | (95% Cl) | value | (95% Cl) | value | (95% Cl) | value | (95% Cl) | value |
| Rs8176318 | AA | 1.06 | 0.92 | 9.48 | 0.04 | 1.90 | 0.22 | 12.19 | 0.02 |
| (BRCA1) | vs | (0.42- | | (1.01- | | (0.68- | | (1.29- | |
| | CC | 2.64) | | 88.80) | | 5.34) | | 115.21) | |
| | AC | 0.56 | 0.05 | 0.58 | 0.21 | 0.70 | 0.31 | 0.49 | 0.16 |
| | vs | (0.32- | | (0.25- | | (0.35- | | (0.18- | |
| | CC | 0.96) | | 1.36) | | 1.39) | | 1.34) | |

Odds ratio (OR) and 95% Confidence interval (CI) according to breast cancer (BC) subtype and race [European American (EA) and African American (AA)] were adjusted in an unconditional logistic regression model. Bolded values show statistical tumor association. Numbers in parenthesis refer to the number of patients in each group (first row).

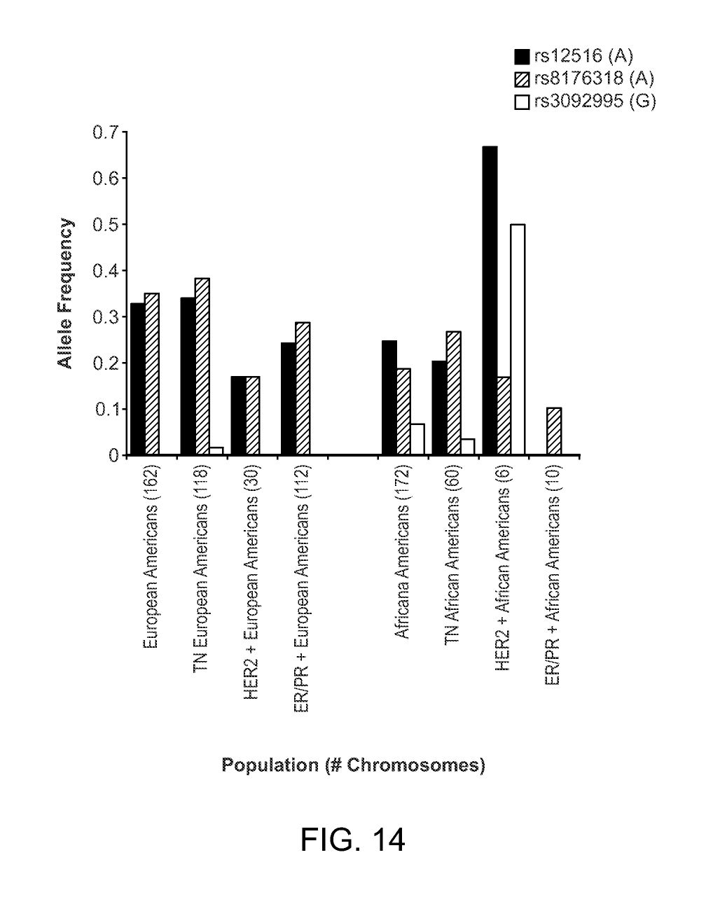

Because BRCA1 dysfunction varies among the breast cancer subtypes, the three 3′UTR SNPs were next evaluated by ethnicity and breast cancer subtype (FIG. 14). It was determined that the homozygous variant form of rs8176318 was significantly associated with risk for TN breast cancer among African American women [OR, 12.19; 95% CI, 1.29-115.21, p=0.02). No association was observed for any of the other SNPs or for ER/PR+ or HER2+ breast cancer subtypes (Table 10).

TABLE 10
The BRCA1 3′UTR SNP rs8176318 and breast cancer association by ethnicity and breast cancer subtype

| | | BC EA (165) | | BC AA (40) | | TN EA ( [symbol] ) | | TN AA ( [symbol] ) | | [symbol] | | ERPRt AA | | ERPRt EA | | ERPRt AA | |
|---|---|---|---|---|---|---|---|---|---|---|---|---|---|---|---|---|---|
| | | vs Control | | vs Control | | vs Control | | vs Control | | [symbol] vs Control | | [symbol] vs Control | | [symbol] vs Control | | [symbol] vs Control | |
| | | EA (92) | | AA (102) | | EA (92) | | AA (102) | | EA (92) | | AA (102) | | EA (92) | | AA (102) | |
| | | OR | | OR | | OR | | OR | | OR | | OR | | OR | | OR | |
| | | (95% | P | (95% | P | (95% | P | (95% | P | (95% | P | (95% | P | (95% | P | (95% | P |
| SNP (gene) | | Cl) | value | Cl) | value | Cl) | value | Cl) | value | Cl) | value | Cl) | value | Cl) | value | Cl) | value |
| Rs8176318 | AA | 1.06 | 0.92 | 9.48 | 0.04 | 1.90 | 0.22 | 12.19 | 0.02 | [symbol] | 1 | NA | 1 | [symbol] | 0.71 | NA | 1 |
| (BRCA1) | vs | (0.42- | | (1.01- | | (0.68- | | (1.29- | | [symbol] | | | | [symbol] | | | |
| | CC | 2.64) | | 88.80) | | 5.34) | | 115.21) | | [symbol] | | | | [symbol] | | | |
| | AC | 0.56 | 0.05 | 0.58 | 0.21 | 0.70 | 0.31 | 0.49 | 0.16 | [symbol] | [symbol] | 0.87 | 1 | 0.15 | 0.002 | [symbol] | 1 |
| | vs | (0.32- | | (0.25- | | (0.35- | | (0.18- | | [symbol] | | [symbol] | | (0. [symbol] | | [symbol] | |
| | CC | 0.96) | | 1.36) | | 1.39) | | 1.34) | | [symbol] | | [symbol] | | [symbol] | | [symbol] | |

Odds ratio (OR) and 95% Confidence interval (CI) according to breast cancer (BC) subtype and race [European American (EA) and African American (AA)] were adjusted in an unconditional logistic regression model. Bolded values show statistical tumor association. Numbers in parenthesis refer to the number of patients in each group (first row). NA is used here in situations where there was no representation of the genotype in the tumor subtype, most likely a result of the small number of patients making up the group. [symbol]
indicates data missing or illegible when filed

### BRCA1 Haplotype Evolution and Frequencies

To better evaluate the BRCA1 region, we added five additional previously reported tagging SNPs (Kidd J R, et al., (abstract/program #58). Presented at the 53rd Annual Meeting of The American Society of Human Genetics, Nov. 4-8, 2003, Los Angeles, Calif. 2003) surrounding the three 3′UTR SNPs we identified in our breast cancer patients. The eight SNPs in total span 267 kb (Table 2). This entire region has high LD and heterozygosities among all eight SNPs composing our haplotype are generally high (30-50%) (http://alfred.med.yale.edu) (Cheung K H, et al. Nucleic acids research 2000; 28(1):361-3; Kidd J R, et al. (abstract/program #58). Presented at the 53rd Annual Meeting of The American Society of Human Genetics, Nov. 4-8, 2003, Los Angeles, Calif. 2003).

These eight SNPs were used to generate global haplotype frequencies (FIG. 8). All of the common haplotypes observed can be explained by accumulation of variation on the ancestral haplotype (FIG. 7). Most of the directly observed haplotypes can be ordered, differing by one derived nucleotide change; in one case two changes are required and in another case a recombination is observed. Collectively, these generate three branches, each starting with a single nucleotide change from the ancestral haplotype. Of note, it was determined that haplotype diversity is much higher in Africa (with 6-9 haplotypes represented) versus outside of Africa (with 3-5 haplotypes). The ancestral haplotype GGCCACTA (SEQ ID NO: 8) is found almost exclusively throughout Africa. The most common haplotype, AGCCATTA (SEQ ID NO: 2) found globally, is very frequent in all populations outside of Africa.

### BRCA1 Haplotypes in Breast Cancer Patients

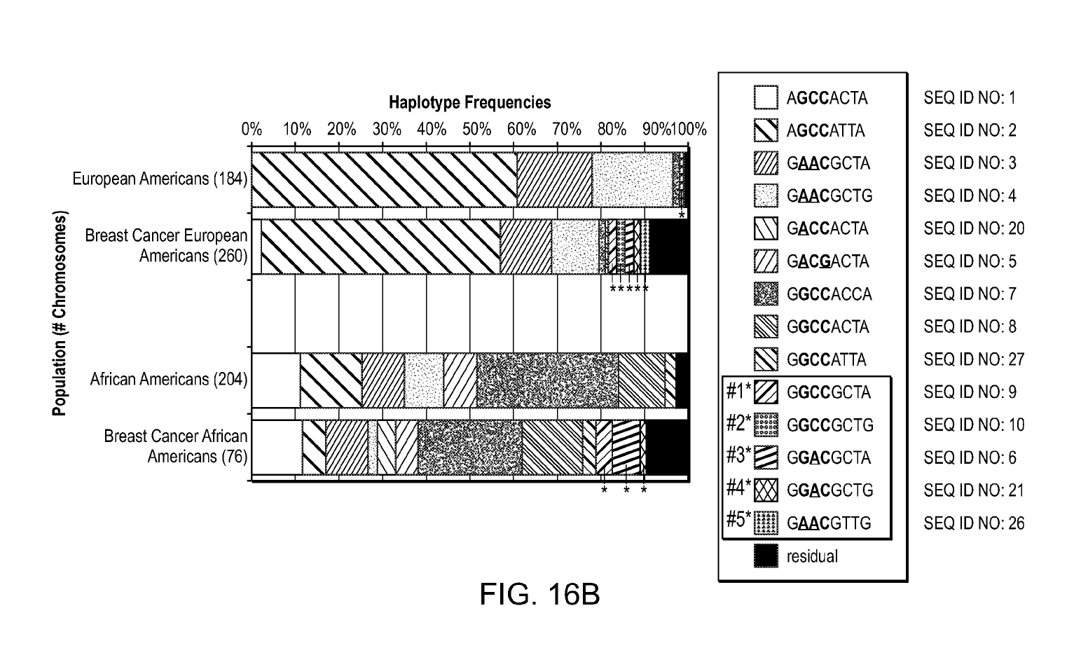

Haplotypes consisting of these eight SNPs in the breast cancer patients were further studied to determine if there were differences in these BRCA1 haplotypes between non-cancerous patients and breast cancer patients. Five haplotypes (GGCCGCTA [SEQ ID NO: 9, #1], GGCCGCTG [SEQ ID NO: 10, #2], GGACGCTA [SEQ ID NO: 6, #3], GGACGCTG [SEQ ID NO: 21, #4], and GAACGTTG [SEQ ID NO: 26, #5]) were identified, which were highly enriched in our breast cancer populations (42/442 total breast cancer chromosomes evaluated), but extremely rare in global control populations. In the global sample of 4500 non-cancerous chromosomes the GGACGCTA (SEQ ID NO: 6) haplotype (#3) was observed on 3 chromosomes and the GGACGCTG (SEQ ID NO: 21) haplotype (#4) was present on 2 chromosomes, while the GGCCGCTA (SEQ ID NO: 9) (#1), GGCCGCTG (SEQ ID NO: 10) (#2) and GAACGTTG (SEQ ID NO: 26) (#5) haplotypes were not seen (<0.1%). This represents an overall global frequency of 0.1% for these haplotypes in non-cancerous controls versus a frequency of 9.50% for breast cancer patient chromosomes (p<0.0001) (FIG. 16A). Two haplotypes (#3 and #4, respectively) are characterized by the derived allele A within the 3′UTR at SNP rs8176318. A third rare haplotype (GAACGTTG (SEQ ID NO: 26), #5) has derived alleles (A) at two of the 3′UTR polymorphisms, rs8176318 and rs12516.

Because the study results demonstrated that these haplotypes varied by ethnicity, to better compare these rare breast cancer haplotypes with the appropriate ethnic populations, breast cancer patients and controls matched were further evaluated by ethnicity. The ethnicity-matched controls were composed of a total of 194 individuals (102 African Americans and 92 European Americans, including a cohort of Yale control Caucasian Americans and African Americans). It was determined that 8.84% of Caucasian American breast cancer patients and 11.84% of African American breast cancer patients contain the rare haplotypes, and again, these haplotypes were rarely found in ethnicity matched controls, with only GGACGCTA (SEQ ID NO: 6) haplotype (#3) found on one European American control chromosome (0.26%, 1/388 chromosomes, p<0.0001, FIG. 16B, Table 8).

TABLE 8
Breast cancer patients studied with rare haplotypes

| | Breast Cancer | Age of | | | SEQ |
|---|---|---|---|---|---|
| Population | Subtype | Onset | Ethnicity | Haplotype | ID NO: |
| Breast Cancer | Triple Negative | 39 | European American | GGCCGCTA | 9 |
| | Triple Negative | 45 | European American | GGCCGCTA | 9 |
| | Triple Negative | 41 | African American | GGCCGCTA | 9 |
| | Triple Negative | 46 | African American | GGCCGCTA | 9 |
| | Triple Negative | 71 | African American | GGCCGCTA | 9 |
| | Triple Negative | NK | NK | GGCCGCTA | 9 |
| | Triple Negative | 34 | European American | GGCCGCTA | 9 |
| | HER2+ | 48 | European American | GGCCGCTA | 9 |
| | ER+/PR+ | 51 | European American | GGCCGCTA | 9 |
| Breast Cancer | Triple Negative | 65 | European American | GGCCGCTG | 10 |
| | Triple Negative | 45 | European American | GGCCGCTG | 10 |
| | Triple Negative | NK | NK | GGCCGCTG | 10 |
| | Triple Negative | NK | NK | GGCCGCTG | 10 |
| | ER+/PR+ | 43 | European American | GGCCGCTG | 10 |
| | ER+/PR+ | 74 | European American | GGCCGCTG | 10 |
| Breast Cancer | Triple Negative | 40 | African American | GGACGCTA | 6 |
| | Triple Negative | 67 | African American | GGACGCTA | 6 |
| | Triple Negative | 61 | African American | GGACGCTA | 6 |
| | Triple Negative | 33 | African American | GGACGCTA | 6 |
| | Triple Negative | 52 | Other | GGACGCTA | 6 |
| | Triple Negative | 52 | Other | GGACGCTA | 6 |
| | Triple Negative | 44 | Other | GGACGCTA | 6 |
| | ER+/PR+ | 76 | European American | GGACGCTA | 6 |
| | ER+/PR+ | 61 | European American | GGACGCTA | 6 |
| | ER+/PR+ | 47 | European American | GGACGCTA | 6 |
| | ER+/PR+ | 34 | European American | GGACGCTA | 6 |
| | ER+/PR+ | 78 | European American | GGACGCTA | 6 |
| | ER+/PR+ | 51 | European American | GGACGCTA | 6 |
| | ER+/PR+ | 82 | African American | GGACGCTA | 6 |
| Control | NK | NK | Cambodians | GGACGCTA | 6 |
| | NK | NK | European Jews | GGACGCTA | 6 |
| | NK | NK | European American | GGACGCTA | 6 |
| Breast Cancer | Triple Negative | 61 | European American | GGACGCTG | 21 |
| | Triple Negative | 34 | European American | GGACGCTG | 21 |
| | Triple Negative | 52 | European American | GGACGCTG | 21 |
| | Triple Negative | 52 | European American | GGACGCTG | 21 |
| | Triple Negative | 72 | African American | GGACGCTG | 21 |
| | Triple Negative | NK | NK | GGACGCTG | 21 |
| | Triple Negative | NK | NK | GGACGCTG | 21 |
| Control | NK | NK | Samaritans | GGACGCTG | 21 |
| | NK | NK | Ticuna | GGACGCTG | 21 |
| Breast Cancer | Triple Negative | 65 | European American | GAACGTTG | 26 |
| | Triple Negative | 60 | European American | GAACGTTG | 26 |
| | Triple Negative | 52 | European American | GAACGTTG | 26 |

List of breast cancer patients and controls with 5 rare haplotypes. Age of onset and ethnicity are listed where available.
NK = information not available or not known. Samples are from both normal tissue and tumor.

### BRCA1 Haplotypes in Breast Cancer Patients by Breast Cancer Subtype

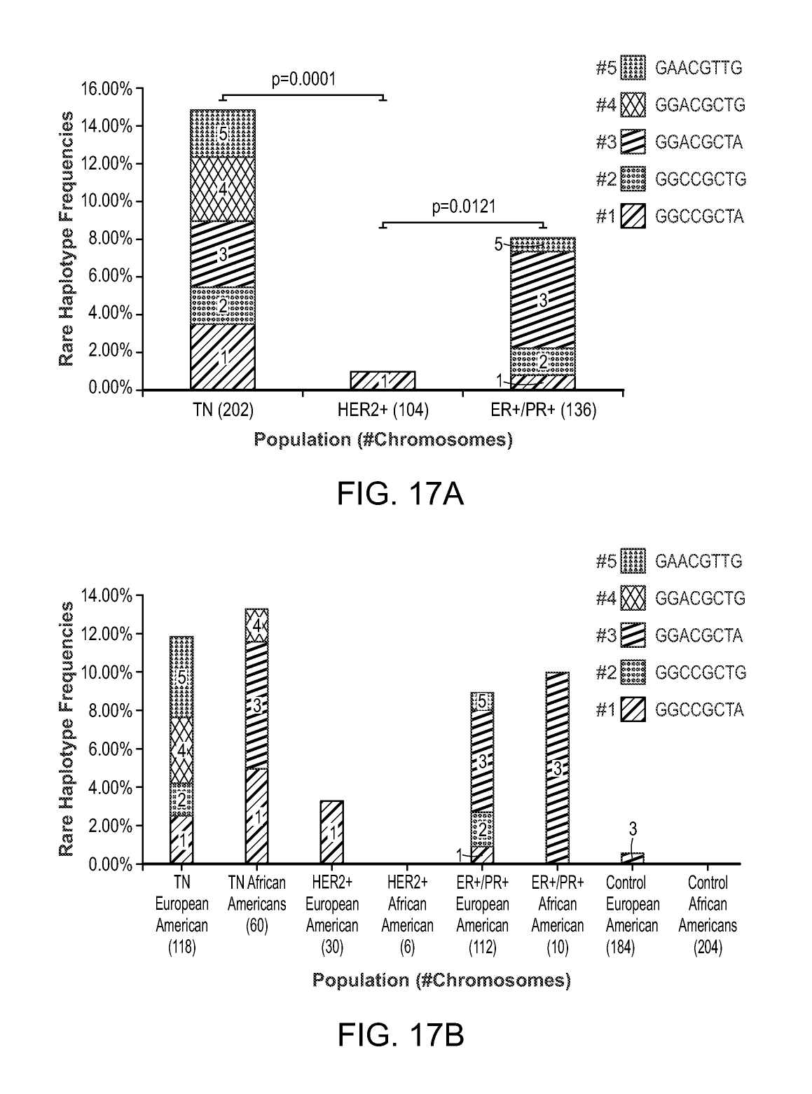

Since known BRCA1 coding sequence mutations vary with breast cancer subtype, it was next determined how the rare haplotypes were distributed amongst breast cancer subtypes. Rare haplotypes varied significantly between the TN, ER/PR+ and HER2+ subtypes, with the TN subgroup harboring these rare haplotypes at the highest rate, at 14.85% (30/202 chromosomes, p=0.014 compared to the others), the ER/PR+ breast cancer subtype next at 8.09% (11/136 ER/PR+ chromosomes), and the HER2+ subtype the least at 1% (1/104), (FIG. 17A, Table 9). The GGACGCTG (SEQ ID NO: 21) haplotype (#4) was only associated with TN tumors and not with the other tumor subtypes. The rare haplotypes were then evaluated by both ethnicity and breast tumor subtype (FIG. 17B). Two haplotypes (#2 and #5, respectively) were unique to breast cancer European Americans. Interestingly, the TN subgroup has the highest proportion of residual haplotypes (9.9%). Residual is defined as the sum of all haplotypes that have a frequency of less than 1% in all populations studied. These findings indicate that the TN subtype of breast cancer has the highest amount of variability throughout this region and is most strongly associated with the rare haplotypes.

TABLE 9
BRCA1 common haplotypes display variation between European and 74
African American breast cancer cases and their ethnicity matched controls.

| | | | Breast Cancer | | | Breast Cancer | |
|---|---|---|---|---|---|---|---|
| | SEQ | European | European | | African | African | |
| | ID | Americans | Americans | P- | Americans | Americans | P- |
| Haplotype | NO: | (184) | (260) | value | (204) | (76) | value |
| AGCCACTA | 1 | 0 | 6 | 0.086 | 23 | 9 | 0.888 |
| AGCCATTA | 2 | 112 | 143 | 0.218 | 29 | 4 | 0.039 |
| GAACGCTA | 3 | 32 | 30 | 0.080 | 20 | 7 | 0.888 |
| GAACGCTG | 4 | 34 | 28 | 0.021 | 18 | 2 | 0.074 |
| GACCACTA | 20 | 0 | 0 | 1.000 | 0 | 3 | 0.019 |
| GACGACTA | 5 | 0 | 0 | 1.000 | 15 | 4 | 0.538 |
| GGCCACCA | 7 | 3 | 4 | 1.000 | 67 | 18 | 0.138 |
| GGCCACTA | 8 | 0 | 2 | 0.514 | 22 | 11 | 0.396 |
| GGCCATTA | 27 | 0 | 0 | 1.000 | 5 | 2 | 1.000 |
| GGCCGCTA | 9 | 0 | 5 | 0.080 | 0 | 3 | 0.019 |
| GGCCGCTG | 10 | 0 | 4 | 0.145 | 0 | 0 | 1.000 |
| GGACGCTA | 6 | 1 | 6 | 0.248 | 0 | 5 | 0.001 |
| GGACGCTG | 21 | 0 | 4 | 0.145 | 0 | 1 | 0.271 |
| GAACGTTG | 26 | 0 | 6 | 0.086 | 0 | 0 | 1.000 |
| ″RESIDUAL″ | * | 2 | 22 | 0.001 | 5 | 7 | 0.020 |

European and African American breast cancer patients were evaluated for haplotype frequency variations as compared to ethnicity-matched controls. Nine common haplotypes are shown. Five additional rare haplotypes among controls but common in breast cancer patients are also listed. The remaining haplotypes with non-zero estimates are combined and listed as RESIDUAL. Values are considered significant if p < 0.05.
*The ″residual″ haplotype in this table was not assigned a sequence identifier because it represents the cumulative estimates of all non-zero haplotypes that are not specifically named, and, therefore, does not represent a single sequence.

### BRCA1 Haplotypes by Age and BRCA Mutation Status

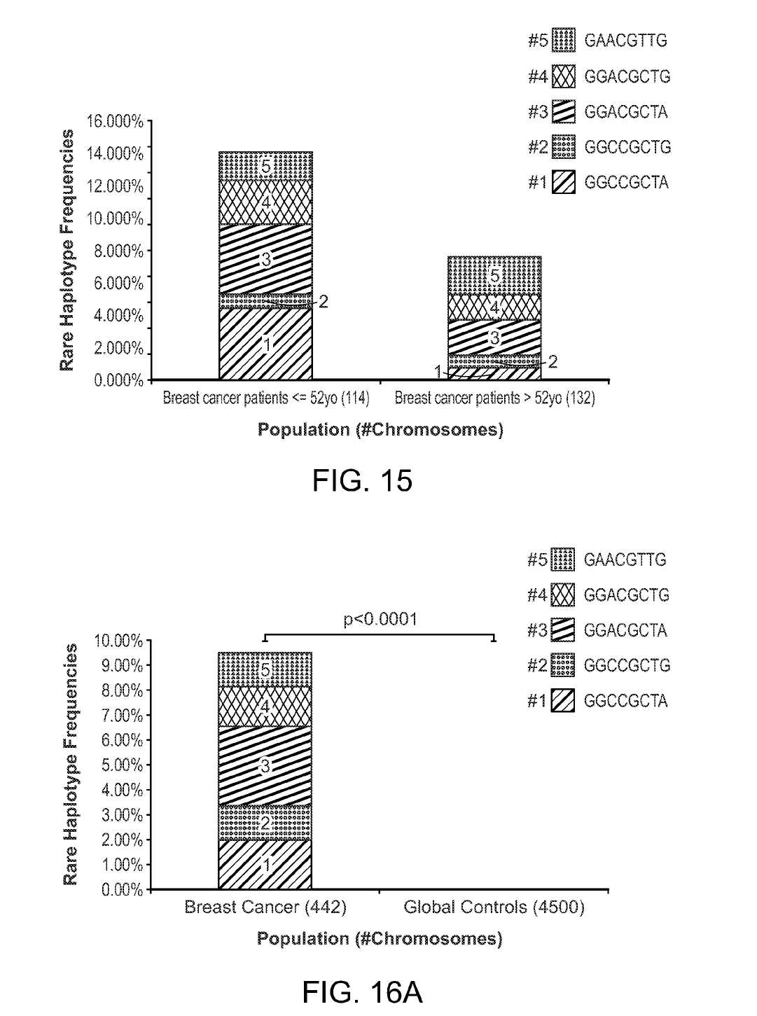

The rare haplotypes were evaluated by age to determine whether younger (premenopausal) women have a higher proportion of these rare haplotypes as compared to post-menopausal women. The rare haplotypes are found more frequently in breast cancer patients under the age of 52; however, this trend was not statistically significant (FIG. 15).

It was also determined whether the rare BRCA1 haplotypes were associated with BRCA1 coding sequence mutations, yet BRCA1 mutation status was unknown for the patients tested in this study. Therefore, a separate cohort of 129 unrelated

European breast cancer patients heterozygous for BRCA1 coding region mutations were tested for the presence of our rare BRCA1 haplotypes. Only one BRCA1 coding sequence mutant patient had a rare haplotype (0.8%, GAACGTTG (SEQ ID NO: 26), #5). The remaining four rare haplotypes were not found in this cohort of patients, suggesting that these rare BRCA1 haplotypes are not surrogate markers of common BRCA1 coding sequence mutations, but rather, these rare BRCA1 haplotypes are unique and novel biomarkers of BRCA1 alterations associated with breast cancer.

### Discussion

This study determined that 299 breast cancer patients harbor five rare BRCA1 haplotypes not commonly found in control populations. These haplotypes include BRCA1 3′UTR SNPs, one of which (rs8176318) shows significant cancer association among African Americans (p=0.04), and, furthermore, is a risk factor for triple negative breast cancer among African Americans (p=0.02) as compared to their ethnicity matched controls. These haplotypes are not associated with common BRCA1 coding region mutations. These findings demonstrate that the rare BRCA1 haplotypes represent new genetic markers of an increased risk of developing breast cancer, as well as non-coding sequence variations in BRCA1 that impact BRCA1 function and lead to increased breast cancer risk.

There have been previous studies conducting haplotype analysis in the BRCA1 region to determine their association with sporadic breast cancer, however, these previous investigators have met with little success (Cox D G, et al. Breast Cancer Res 2005; 7(2):R171-5; Freedman M L, et al. Cancer research 2005; 65(16):7516-22).

This study is the first BRCA1 haplotype study of sporadic breast cancer that includes rare functional variants in the 3′UTR noncoding regulatory regions of BRCA1 as part of the haplotype analysis. Evidence is fast becoming available to support the theory that variants within the 3′UTR increase susceptibility to cancer through gene expression control (Chin L I, et al. Cancer research 2008; 68(20):8535-40; Landi D, et al. Carcinogenesis 2008; 29(3):579-84). While we are unable to determine if in the rare haplotypes the increased breast cancer risk is one single variant within the haplotype or a combination of alleles, it is hypothesized that the combination of the functional 3′UTR variants with the other variants comprising each haplotype is predictive of meaningful BRCA1 dysfunction.

Sporadic breast cancer was further analyzed by subtype in our haplotype analysis. Because breast cancers resulting from BRCA1 mutations are most frequently associated with TN (57%) (Atchley D P, et al. J Clin Oncol 2008; 26(26):4282-8) and ER+ breast cancers (34%) (Tung N, et al. Breast Cancer Res; 12(1):R12), and are rarely found in HER2+ breast cancers (about 3%) (Lakhani S R, et al. J Clin Oncol 2002; 20(9):2310-8), our findings that the rare haplotypes are primarily in TN and ER+ breast cancer further supports our hypothesis that they are associated with true BRCA1 dysfunction.

Future studies will focus on some of the individual SNPs within our BRCA1 haplotype. Of particular interest is the tagging SNPs rs1060915, a BRCA1 synonymous exonic mutation, with the derived allele G in all five rare haplotypes. Rs1060915 is a variant of unknown significance (VUS). The Breast Cancer Information Core (BIC) classifies this VUS as neutral or of little clinical importance based on mRNA and protein levels produced based on comparison to wild type sequence (http://research.nhgri.nih.gov/bic/). Although Myriad Genetics, Inc., has associated this SNP with high-risk women and classifies it as polymorphic because as it is seen commonly in their high-risk patient cohort, in contrast to this study, they have not assigned this SNP a role as a biomarker of increased risk for developing breast or ovarian cancer. Specifically, Myriad has not shown rs1060915 to be a significant predictor of a subject's risk of developing the TN subtype of breast cancer.

Recently, similar coding sequence SNPs in BRCA1 have been shown to be located in miRNA binding sites and can influence tumor Susceptibility (Nicoloso M S, et al. Cancer research; 70(7):2789-98). 3′UTR SNPs leading to miRNA disruption in combination with exonic SNPs that impact miRNA binding are one mechanism leading to increased breast cancer risk in the rare haplotypes.

The enrichment of the rare haplotypes in the TN subtype of breast cancer is especially striking. Not only does this subtype statistically associate with our rare haplotypes as compared to controls (p<0.0001), but TN breast cancer is also the most common subtype associated with our rare haplotypes. Risk factors for TN breast cancer are unlike other forms of breast cancer because TN tumors are not associated with estrogen stimulation (nulliparity, obesity, hormone replacement therapy). The disassociation of TN to estrogen stimulation strongly suggests that there are additional genetic causes. Because TN breast cancers have the worst outcome, it is perhaps most important to identify those at risk of developing this subtype of breast cancer.

Limitations of our studies may include the small number of patients harboring the rare haplotypes, preventing potential significant associations with age and race to be uncovered. Additionally, the cohort of European breast cancer patients heterozygous for BRCA1 coding region mutations are mostly Western European Caucasian, with a small percentage possibly of mixed European descent. The ethnically narrow group may have limited the findings of the rare haplotypes among BRCA1 mutation carriers. However, the high association of the rare haplotypes with breast cancer makes these findings even more strongly statistically significant. This study provides evidence that these rare haplotypes can be used as genetic markers of an increased risk of developing breast cancer and supports future work to validate the results in larger sample sizes as well as to further elucidate the biological function of these haplotypes and their mechanisms of increased breast cancer risk.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A BRCA1 haplotype comprising at least one single nucleotide polymorphism (SNP), wherein the presence of the SNPs increases a subject's risk of developing breast or ovarian cancer.

2. The haplotype of claim 1, wherein each of the SNP alters the activity of one or more miRNA(s).

3. The haplotype of claim 1, wherein the SNP is located in a noncoding or a coding region of the BRCA1 gene.

4. The haplotype of claim 2, wherein the SNP is located in a noncoding or a coding region of the BRCA1 gene.

5. The haplotype of claim 1 or 4, wherein the SNP is selected from the group consisting of ra9911630, rs12516, rs8176318, rs3092995, rs1060915, rs799912, rs9908805, and rs17599948.

6. The haplotype of claim 1, wherein the SNP is selected from the group consisting of rs12516, rs8176318, rs3092995, rs1060915, and rs799912.

7. The haplotype of claim 1, wherein the SNP is rs8176318 or rs1060915.

8. The haplotype of claim 1, wherein the haplotype comprises rs8176318 and rs1060915.

9. The haplotype of claim 1, wherein the presence of the SNP increases a subject's risk of developing triple negative (TN) breast cancer.

10. The haplotype of claim 1, wherein the haplotype comprises the nucleotide sequence of GGACGCTA (SEQ ID NO: 6), GGCCGCTA (SEQ ID NO: 9), GGCCGCTG (SEQ ID NO: 10), GGACGCTG (SEQ ID NO: 21), or GAACGTTG (SEQ ID NO: 26).

11. A BRCA1 polymorphic signature that indicates an increased risk for developing breast or ovarian cancer, the signature comprising the determination of the presence or absence of the following single nucleotide polymorphisms (SNPs) rs8176318 and rs1060915, wherein the presence of these SNPs indicates an increased risk for developing breast or ovarian cancer.

12. The signature of claim 11, wherein the signature further comprises the determination of the presence or absence of at least one SNP selected from the group consisting of rs12516, rs3092995, and rs799912.

13. The signature of claim 11 or 12, wherein the signature further comprises the determination of the presence or absence of at least one SNP selected from the group consisting of rs9911630, rs9908805, and rs17599948.

14. The signature of claim 11, wherein rs8176318 and rs1060915 alter the binding efficacy of at least one microRNA (miRNA).

15. The signature of claim 12, wherein rs12516, rs3092995, or rs799912, alters the binding efficacy of at least one miRNA.

16. The signature of claim 11, wherein the at least one miRNA is miR-7.

17. The signature of claim 1, wherein the signature further comprises the identification of the presence or absence of a SNP in the BRCA1 gene that alters the binding efficacy of one or more microRNAs.

18. The signature of claim 17, wherein the SNP occurs within a coding or a non-coding region.

19. The signature of claim 18, wherein the non-coding region is a 3′ untranslated region (UTR), an intron, an intergenic region, a cis-regulatory element, promoter element, enhancer element, or a 5′ untranslated region (UTR).

20. The signature of claim 18, wherein the coding region is an exon.

21. The signature of claim 11, wherein the breast cancer is triple negative breast cancer.

22. A method of identifying a SNP that decreases expression of the BRCA1 gene and increases a subject's risk of developing breast or ovarian cancer, comprising:
(a) obtaining a sample from a test subject;
(b) obtaining a control sample;
(c) determining the presence or absence of a SNP in at least one miRNA binding site within a DNA sequence from the test sample; and
(d) evaluating the binding efficacy of at least one miRNA to the at least one miRNA binding site containing the SNP compared to the binding efficacy of the miRNA to the same miRNA binding site in corresponding DNA sequence from the control sample,
wherein the presence of a statistically-significant alteration in the binding efficacy of the at least one miRNA to the corresponding binding site(s) between the control and test samples indicates that the presence or absence of the SNP inhibits miRNA-mediated protection or increases miRNA-mediated repression of BRCA1 gene expression, thereby identifying a SNP that also increases a subject's risk of developing breast or ovarian cancer.

23. A method of identifying a SNP that decreases expression of the BRCA1 gene and increases a subject's risk of developing breast or ovarian cancer, comprising:
(a) obtaining a sample from a test subject;
(b) determining the presence or absence of a SNP in at least one miRNA binding site in a DNA sequence from the test sample; and
(c) evaluating the prevalence of the SNP within a breast or ovarian cancer population with respect to the expected prevalence of the SNP in one or more world population(s),
wherein a statistically-significant increase in the presence or absence of the SNP in the tumor sample compared to the one or more world populations indicates that the SNP is positively associated with an increased risk of developing breast or ovarian cancer and wherein the presence or absence of the SNP within at least one miRNA binding site that decreases expression of BRCA1 indicates that the presence or absence of the SNP inhibits miRNA-mediated protection or increases miRNA-mediated repression of BRCA1 gene expression, thereby identifying a SNP that also increases a subject's risk of developing breast or ovarian cancer.

24. The method of claim 22 or 23, wherein the test subject has been diagnosed with breast or ovarian cancer.

25. The method of claim 22, wherein the control sample is obtained from a subject who has not been diagnosed with any cancer.

26. The method of claim 22 or 23, wherein the miRNA binding site is determined empirically, identified in a database, or predicted using an algorithm.

27. The method of claim 22 or 23, wherein the presence or absence of the SNP is determined empirically, identified in a database, or predicted using an algorithm.

28. The method of claim 22, wherein the binding efficacy is evaluated in vitro or ex vivo.

29. The method of claim 22 or 23, wherein the breast cancer is sporadic or inherited.

30. The method of claim 22 or 23, wherein the ovarian cancer is sporadic or inherited.

31. A method of identifying a subject at risk of developing breast or ovarian cancer, comprising,
a) obtaining a DNA sample from a test subject; and
b) determining the presence of at least one SNP selected from the group consisting of rs12516, rs8176318, rs3092995, and rs799912 in at least one DNA sequence from the sample,
wherein the presence of the at least one SNP in the at least one DNA sequence increases the subject's risk of developing breast or ovarian cancer 10-fold compared to a normal subject.

32. The method of claim 31, further comprising the step of determining the presence of rs1060915, wherein the combined presence of rs1060915 and at least one SNP selected from the group consisting of rs12516, rs8176318, rs3092995, and rs799912 in the at least one DNA sequence increases the subject's risk of developing breast or ovarian cancer 100-fold compared to a normal subject.

33. The method of claim 31, wherein a normal subject is a subject who does not carry rs12516, rs8176318, rs3092995, rs799912, or rs1060915.

34. The method of claim 31, wherein the breast cancer is sporadic or inherited.

35. The method of claim 31, wherein the ovarian cancer is sporadic or inherited.

36. A method of identifying a subject at risk of developing triple negative (TN) breast cancer, comprising,
a) obtaining a DNA sample from a test subject; and
b) determining the presence of rs8176318 or rs1060915 in at least one DNA sequence from the sample,
wherein the presence of rs8176318 or rs1060915 in the at least one DNA sequence increases the subject's risk of developing TN breast cancer compared to a normal subject.

37. The method of claim 36, comprising the step of determining the presence of rs8176318 and rs1060915, wherein the combined presence of rs1060915 and rs8176318 in the at least one DNA sequence further increases the subject's risk of developing TN breast cancer.

38. The method of claim 36 or 37, wherein the breast cancer is sporadic or inherited.

39. The method of claim 36 or 37, wherein the ovarian cancer is sporadic or inherited.

40. The method of claim 36 or 37, wherein the test subject is African American.

41. The signature of claim 13, wherein rs9911630, rs9908805, or rs17599948 alters the binding efficacy of at least one miRNA.


SEQUENCE LISTING (43 sequences)

SEQ ID NO: 1 - DNA, 8 bp, Homo sapiens
agccacta

SEQ ID NO: 2 - DNA, 8 bp, Homo sapiens
agccatta

SEQ ID NO: 3 - DNA, 8 bp, Homo sapiens
gaacgcta

SEQ ID NO: 4 - DNA, 8 bp, Homo sapiens
gaacgctg

SEQ ID NO: 5 - DNA, 8 bp, Homo sapiens
gacgacta

SEQ ID NO: 6 - DNA, 8 bp, Homo sapiens
ggacgcta

SEQ ID NO: 7 - DNA, 8 bp, Homo sapiens
ggccacca

SEQ ID NO: 8 - DNA, 8 bp, Homo sapiens
ggccacta

SEQ ID NO: 9 - DNA, 8 bp, Homo sapiens
ggccgcta

SEQ ID NO: 10 - DNA, 8 bp, Homo sapiens
ggccgctg

SEQ ID NO: 11 - DNA, 7224 bp, Homo sapiens
gtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 12 - DNA, 7287 bp, Homo sapiens
gtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagattcgcatatacatggccaaaggaacaactccatgttttctaaaaggcctagagaacatatatcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 13 - DNA, 7132 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 14 - DNA, 3699 bp, Homo sapiens
ttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 15 - DNA, 3800 bp, Homo sapiens
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 16 - DNA, 7128 bp, Homo sapiens
agataactgggcccctgcgctcaggaggccttcaccctctgctctgggtaaaggtagtagagtcccgggaaagggacagggggcccaagtgatgctctggggtactggcgtgggagagtggatttccgaagctgacagatggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaaaaaaaaaaaaaaaaaa

SEQ ID NO: 17 - DNA, 9 bp, Homo sapiens
gaacagata

SEQ ID NO: 18 - DNA, 9 bp, Homo sapiens
gaacagata

SEQ ID NO: 19 - DNA, 8 bp, Homo sapiens
agccgctg

SEQ ID NO: 20 - DNA, 8 bp, Homo sapiens
gaccacta

SEQ ID NO: 21 - DNA, 8 bp, Homo sapiens
ggacgctg

SEQ ID NO: 22 - DNA, 20 bp, Homo sapiens
gagctggacacctacctgat

SEQ ID NO: 23 - DNA, 19 bp, Homo sapiens
gagaaagtcggctggccta

SEQ ID NO: 24 - DNA, 20 bp, Homo sapiens
cctacctgataccccagatc

SEQ ID NO: 25 - DNA, 22 bp, Homo sapiens
ggcctaagtctcaagaacagtc

SEQ ID NO: 26 - DNA, 8 bp, Homo sapiens
gaacgttg

SEQ ID NO: 27 - DNA, 8 bp, Homo sapiens
ggccatta

SEQ ID NO: 28 - RNA, 34 nt, Homo sapiens
aacagcuacccuuccaucauaagugacucuucug

SEQ ID NO: 29 - RNA, 23 nt, Homo sapiens
uggaagacuagugauuuuguugu

SEQ ID NO: 30 - RNA, 27 nt, Homo sapiens
auaagugacuccucugcccuugaggac

SEQ ID NO: 31 - RNA, 21 nt, Homo sapiens
cuuuuugcggucugggcuugc

SEQ ID NO: 32 - RNA, 49 nt, Homo sapiens
ugggagccagccuucuaacagcuacccuuccaucauaagugacucuucu

SEQ ID NO: 33 - RNA, 22 nt, Homo sapiens
uggagagaaaggcaguuccuga

SEQ ID NO: 34 - RNA, 53 nt, Homo sapiens
augggagccagccuucuaacagcuacccuuccaucauaagugacucuucugcc

SEQ ID NO: 35 - RNA, 24 nt, Homo sapiens
agcagaagcagggagguucuccca

SEQ ID NO: 36 - DNA, 52 bp, Homo sapiens
ggaggggaggcggctgcccacccttcracactattacccatcggtcacttgt

SEQ ID NO: 37 - DNA, 52 bp, Homo sapiens
aaggactaggtcttccctagccccccyagtgtgcaagggcagtgaagacttg

SEQ ID NO: 38 - DNA, 52 bp, Homo sapiens
cttctctaaaaccctgtgttcacaaakgcagagagtcagacccttcaatgga

SEQ ID NO: 39 - DNA, 52 bp, Homo sapiens
gccacaggtacagagccacaggacccsaagaatgagcttacaaagtggcctt

SEQ ID NO: 40 - DNA, 60 bp, Homo sapiens
aacagctacccttccatcataagtgactcytctgcccttgaggacctgcraaatccagaa

SEQ ID NO: 41 - DNA, 52 bp, Homo sapiens
cattgacatctgtataaaccgtgtgayggcagtgatttagtaactttttgtc

SEQ ID NO: 42 - DNA, 52 bp, Homo sapiens
gtcacgtttttttccccccctctacaytgcagatgtggctcccaatgttgac

SEQ ID NO: 43 - DNA, 52 bp, Homo sapiens
gtagggaaaatgctggagccttgactrtaatgtaaaccattctccccaaaag